(19) United States
(12) Patent Application Publication
(10) Pub. No.: US 2026/0191777 A1
(43) Pub. Date: Jul. 9, 2026

(54) STABLE EDARAVONE LIQUID FORMULATIONS AND METHOD OF ITS USE

(71) Applicant: Sandipkumar Arvindbhai Patel, Robbinsville, NJ (US)

(72) Inventor: Sandipkumar Arvindbhai Patel, Robbinsville, NJ (US)

(21) Appl. No.: 19/440,626

(22) Filed: Jan. 5, 2026

Related U.S. Application Data
(60) Provisional application No. 63/741,871, filed on Jan. 4, 2025.
(60) Provisional application No. 63/824,128, filed on Jun. 15, 2025.
(60) Provisional application No. 63/946,112, filed on Dec. 21, 2025.

(51) Int. Cl. A61K 9/08; A61K 9/00; A61K 31/4152; A61K 47/02; A61K 47/10; A61K 47/18

(52) U.S. Cl. CPC A61K 9/08; A61K 9/0053; A61K 9/0095; A61K 31/4152; A61K 47/02; A61K 47/10; A61K 47/183

(57) ABSTRACT

A Stable liquid pharmaceutical formulation of edaravone or pharmaceutically acceptable salt thereof is provided. The liquid formulation of edaravone comprises edaravone, a solubilizer, and one or more optional pharmaceutically acceptable excipients, wherein the formulation is solution at concentration of at least 5 mg/mL, preferably about 21 mg/mL of edaravone, and comprises very low amount of ethanol. The method of preparing the stable liquid edaravone formulation and the use of such formulation for the treatment of diseases treatable by edaravone is described.

### CROSS REFERENCE TO RELATED APPLICATIONS

This application claims priority to U.S. Provisional Application No. 63/741,871 filed on Jan. 4, 2025, U.S. Provisional Application No. 63/824,128 filed on Jun. 15, 2025, and U.S. Provisional Application No. 63/946,112 filed on Dec. 21, 2025, the disclosures of which are incorporated herein by reference in its entirety.

### FIELD OF THE INVENTION

The present invention relates to stable liquid pharmaceutical formulations of Edaravone or its pharmaceutically acceptable salt thereof and its method of use.

### BACKGROUND OF THE INVENTION

Edaravone is a member of the substituted 2-pyrazolin-5-one class. The chemical name of edaravone is [3-methyl-1-phenyl-2-pyrazolin-5-one]. The molecular formula is C₁₀H₁₀N₂O and the molecular weight is 174.20.

The chemical structure is:

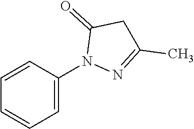

Edaravone is indicated for the treatment of amyotrophic lateral sclerosis (ALS). Edaravone is a white crystalline powder with a melting point of 129.7° C. It is freely soluble in acetic acid, methanol, or ethanol and slightly soluble in water or diethyl ether.

Edaravone is marketed as RADICAVA® injection and RADICAVA® ORS oral suspension.

RADICAVA® Injection is a clear, colorless liquid provided as a sterile solution. RADICAVA® injection is supplied for intravenous infusion in a polypropylene bag containing 30 mg edaravone in 100 mL isotonic, sterile, aqueous solution, which is further overwrapped with polyvinyl alcohol (PVA) secondary packaging. The overwrapped package also contains an oxygen absorber and oxygen indicator to minimize oxidation. Each bag contains the following inactive ingredients: L-cysteine hydrochloride hydrate (10 mg), sodium bisulfite (20 mg). Sodium chloride is added for isotonicity, and phosphoric acid and sodium hydroxide are added to adjust to pH 4.

RADICAVA® ORS (edaravone) oral suspension is a white to off-white color, opaque, homogenous suspension containing 105 mg of edaravone per 5 mL of suspension.

RADICAVA® ORS contains the following inactive ingredients: L-cysteine hydrochloride hydrate, polyvinyl alcohol, simethicone emulsion, sodium bisulfite, sorbitol, and xanthan gum. Phosphoric acid and sodium hydroxide are added to adjust to pH 4.

Current commercial formulations of edaravone for oral administration, such as RADICAVA® ORS, are provided as aqueous suspensions. While suspensions allow for the delivery of hydrophobic drugs, they impose significant logistical and patient compliance burdens due to inherent physical instabilities. For instance, the commercial suspension requires rigorous cold-chain management, mandating storage in an upright position at 2° C. to 8° C. (refrigerated) while at the pharmacy. Even after dispensing, strict handling protocols remain: the product must be stored upright at controlled room temperature (20° C. to 25° C.), protected from light, and discarded within 15 days of opening or 30 days from shipment, whichever occurs first. These strict parameters suggest that even in a pre-dominant solid-state in suspension format, the active pharmaceutical ingredient (API) remains sensitive to environmental stressors.

Furthermore, the thermodynamic instability of suspensions necessitates complex administration steps to ensure dose uniformity. According to the product label, the patient must invert the bottle and shake vigorously for at least 30 seconds prior to every use. This agitation is critical to re-suspend sedimented particles and achieve a temporary homogenous state. Failure to strictly adhere to this agitation protocol—or partial re-suspension—can lead to significant dosing errors, resulting in the administration of sub-therapeutic doses (supernatant) or super-potent doses (sediment), compromising patient safety and efficacy.

Developing a high-concentration oral solution to overcome the disadvantages of a suspension presents a substantial formulation challenge based on the physicochemical properties of edaravone. While stable dilute solutions of edaravone exist (e.g., for intravenous infusion), they utilize large fluid volumes not suitable for oral bolus administration. To formulate a patient-friendly oral solution (e.g., 5-10 mL volume), the concentration of edaravone must be significantly increased. However, edaravone exhibits poor water solubility and is highly prone to oxidative degradation when in the dissolved state.

Parikh et al. (2018), discloses edaravone as a BCS Class IV compound with a “poor aqueous solubility” of approximately 1.85 mg/mL in water. Parikh et al. (2017) further discloses the solubility of edaravone in various vehicles and organic solvents reported as mean±standard deviation (S.D., n=3). For example, propylene glycol (PG) at 5.93±1.63 mg/mL, glycerol at 9.92±1.42 mg/mL, PEG 400 at 6.40±1.47 mg/mL, PEG 300 at 50.63±4.63 mg/mL, and ethanol at 70.28±5.27 mg/mL.

The disclosures by Parikh et al. (2017) established a technical understanding in the art that solvents such as propylene glycol and polyethylene glycol (e.g., PEG 400) lack the intrinsic solvent capacity to support high concentration edaravone solutions at ambient conditions. Specifically, the reported solubility values (approximately 5.93 mg/mL for propylene glycol and 6.40 mg/mL for PEG 400) suggested that these vehicles would be unsuitable for formulating high-concentration dosage forms without significant fractions of ethanol. Consequently, based on conventional solubility models, the ability of these solvents-individually or in simple binary combinations—to achieve stable concentrations exceeding 20 mg/mL (a value more than 3-fold higher than the reported limits) was unpredictable and contrary to the trends established in the prior art.

Although Parikh et al. (2017) reported data suggesting high solubility in polyethylene glycol 300 (PEG 300), patent disclosures by the same authors (e.g., U.S. Pat. No. 11,020,375) notably describe compositions involving PEG 300 without providing enabling data for a stable liquid solution, instead directing the invention toward solid dosage forms (e.g., tablets). This effective abandonment of the liquid format—despite the known solubility potential—indicates that those of skill in the art recognized a significant technical barrier, such as chemical instability (e.g., peroxide formation in PEGs) or insufficient long-term stability, that prevented the successful commercialization of a simple, high-concentration liquid solution.

The extensive patent portfolio of the commercial innovator (Mitsubishi Tanabe Pharma) evidences a lack of viable solution-based methodologies, focusing exclusively on complex suspension formulations (e.g., U.S. Pat. Nos. 10,987,341; 11,241,416; 12,285,409). Significantly, while these documents disclose the use of organic solvents like propylene glycol and polyethylene glycol, they characterize them merely as components of a “dispersion medium” for solid particles rather than as vehicles for thermodynamic solubilization. This perceived difficulty in stabilizing edaravone in organic vehicles is explicitly codified in recent disclosures (e.g., U.S. Pat. Nos. 12,194,025 and 12,310,946), which teach away from the use of solvent-based systems by restricting water-soluble organic solvents—specifically listing propylene glycol and PEG—to negligible levels (“less than 3% by weight”). This restriction implies that the high solvent concentrations required for solubilization were considered detrimental to formulation stability or safety.

Despite commercializing a suspension, the innovator's filings acknowledge a critical unmet need for a solution-based format to address administration complications such as tube clogging; however, the prior art failed to provide a stable solution to meet this need. For instance, the '025 patent describes a dilute “Liquid Edaravone Solution” prepared at alkaline pH to aid dissolution but admits to poor chemical stability by requiring extemporaneous preparation “within 3 hours prior to oral administration.” This limitation confirms that the prior art's reliance on ionization (alkaline pH) or low-solvent strategies yielded only unstable, short-lived preparations, failing to teach a method for achieving a shelf-stable, high-concentration liquid solution where the drug remains chemically stable.

Japanese Patent No. JP4850426B2 (Yamada et al.) discloses an Edaravone injection prepared at about 1.5% w/w to 3.5% w/w (approx. 15 mg/mL to 35 mg/mL) using 40% w/w to 80% w/w ethanol in water. Yamada et al. describes that an edaravone solution prepared in this ethanol concentration range shows no color change when heated at 121° C. for 20 minutes, with no impurity generation observed by high performance liquid chromatography. While the solubility of Edaravone in pure ethanol was reported at 111 mg/mL (25° C.), the solubility in 40% w/w ethanol was only ~21 mg/mL (at 25° C.), which decreased to about 10 mg/mL at 4° C. This drastic reduction underscores the risk of crystallization due to temperature excursions (as reported in FIG. 1 of JP '426). Yamada et al. explicitly anticipated this risk, stating that for a “high concentration edaravone injection solution of 15 to 30 mg/mL . . . an ethanol concentration in the range of 40% w/w to 80% w/w was considered necessary” to prevent precipitation.

U.S. Pat. Nos. 12,491,178, 12,343,330, and U.S. Patent Publication No. 2025/0235431 (Dube et al.) disclose high concentration edaravone solutions relying on ethanol at approximately 40% w/w to 50% v/v. This approach essentially reiterates the teachings of Yamada et al., which explicitly identified the 40% w/w to 80% w/w ethanol range as critical for preventing crystallization and maintaining stability in high-concentration formulations. Although Dube et al. describe the addition of crystallization inhibitors, the core solvent system falls squarely within the range previously disclosed and validated by Yamada et al. Furthermore, despite broadly claiming alternative organic solvents such as propylene glycol, polyethylene glycol, and glycerin, Dube et al. failed to provide enabling working examples utilizing these solvents in the absence of significant amounts of ethanol (e.g., ~40% w/w). Notably, while Dube et al. present solubility data for ‘Capryol 90’ (Propylene Glycol Monocaprylate), a surfactant with high solubilizing capacity, they fail to provide corresponding data for Propylene Glycol itself. This omission obscures the fact that Propylene Glycol has significantly lower solubility (~6 mg/mL) compared to the claimed Ethanol systems, thereby misleading a person of skill in the art regarding the viability of Propylene Glycol as a primary solvent. This lack of data further implies that Dube et al. could not overcome the known technical barriers—such as the low solubility (e.g. propylene glycol) or oxidative instability inherent to polyethylene glycol-without reverting to the high-ethanol “crutch” established by Yamada.

Ethanol is known to harm patients when used at high concentration. It introduces manufacturing challenge due to flammable nature. Moreover, when container is not closed tightly or even multiple opening/closing for several days, evaporation of ethanol may crystalize edaravone as anticipated by Yamada et al. and Dube et al. Such crystallization will end up in inconsistent dosing which lacks whole purpose of making solution.

In the United States, 21 CFR § 328.10 establishes the maximum alcohol concentration limits, and 21 CFR § 328.50 mandates the labeling requirements for alcohol content. According to 21 CFR § 328.10, for children under 6 years, maximum 0.5% alcohol; for children 6 to under 12 years, maximum 5% alcohol, and for adults and children 12 years and over, maximum 10% alcohol is strictly controlled by the government. While these limits apply specifically to OTC products, they reflect the FDA's recognition of ethanol's toxicity. High-concentration ethanol formulations carry a risk of abuse, particularly in patients with a history of alcoholism. A 40% ethanol solution is roughly equivalent to the alcohol content of vodka or whiskey, which poses a relapse risk for recovering alcoholics and is undesirable for pediatric or vulnerable populations.

Furthermore, according to major pharmaceutical interaction databases (e.g., Drugs.com), ethanol interacts with over 500 medications, including 59 major interactions classified as “Highly clinically significant” where the risk outweighs the benefit. For ALS patients who are often on complex polypharmacy regimens, the introduction of a daily high-ethanol load creates an unnecessary burden of adverse events and contraindications. Therefore, there remains an unmet need for a high-concentration Edaravone formulation that achieves stability and solubility without relying on the unsafe 40-50% ethanol levels disclosed by Yamada and Dube.¹ ¹ https://www.drugs.com/drug-interactions/ethanol,ethyl-alcohol-index.html

Thus, prior art presents a problematic dichotomy between liquid and solid formats, where neither offers a satisfactory solution. Existing liquid formulations are either chemically unstable (requiring extemporaneous preparation, e.g., 3-hour shelf life) or toxic (due to high ethanol concentrations), whereas stable solid or suspension forms impose significant burdens on dysphagic patients (e.g., refrigeration, vigorous resuspension, or difficulty swallowing). Consequently, there remains a critical unmet need for a high-concentration, low ethanol liquid solution that combines the shelf-stability of a solid with the dosing precision and ease of administration of a simple liquid.

This challenge is particularly acute given the limitations of the commercial innovator's product. Given that the current commercial suspension—which benefits from the inherent chemical stability of the drug in its solid state—still necessitates refrigeration and possesses a limited shelf-life, it is technically counter-intuitive to formulate a high—concentration solution—where the drug is fully dissolved and maximally reactive—that is stable at room temperature. Thus, there remains an unmet need for a solubilized, high-concentration edaravone formulation that ensures dose homogeneity and chemical stability without the burdens of resuspension, cold-chain storage, or high-ethanol vehicles.

### SUMMARY OF THE INVENTION

The present invention relates to stable, high-concentration, ready-to-use liquid pharmaceutical compositions of edaravone, method of its use, and method of treating diseases treatable by edaravone for patients in need thereof. More specifically, the invention provides a single-phase solution comprising edaravone at a concentration from about 5 mg/mL to about 100 mg/mL, from about 10 mg/mL to about 50 mg/mL, about 15 mg/mL to about 25 mg/mL, about 21 mg/mL solubilized in a vehicle comprising one or more pharmaceutically acceptable, water-miscible organic solvents.

In some embodiments of the present invention, the stable liquid formulation of edaravone is suitable for administration via oral, intranasal and parenteral administration, preferably oral route of administration.

The present inventor has identified a specific solvent architecture that overcomes the solubility and stability limitations taught in the prior art. While prior art disclosures suggest that water-miscible solvents (e.g., propylene glycol) are insufficient to support high drug loading and stability or restricted their use to negligible levels (e.g., less than 3%) the present invention utilizes the same vehicle system to achieve thermodynamic solubilization of edaravone and extended stability.

In an embodiment of the present invention, the stable liquid pharmaceutical composition of edaravone comprises at least one water-miscible organic vehicle selected from the group comprising of propylene glycol, polyethylene glycol, glycerin and mixtures thereof, wherein ethanol is present at substantially low concentration (<10% w/v). In another embodiment of the present invention, the vehicle further comprises water.

In some embodiments of the present invention, the stable liquid pharmaceutical composition of edaravone comprises one or more organic solvent at concentration from about 10% v/v to about 99% v/v, preferably from about 40% v/v to about 90% v/v, preferably about 60% v/v to about 90% v/v, preferably about 80% v/v. In some other embodiments of the present invention, the stable liquid pharmaceutical composition comprises one or more organic solvent at concentration from about 1% v/v to about 50% v/v, preferably from about 5% v/v to about 40% v/v, preferably about 10% v/v to about 30% v/v. In some embodiments of the present invention, ethanol is not a desirable ingredient due to handling risk during manufacturing, safety in pediatrics, risk of drug abuse, potential drug interactions, and potential risk of crystallization due to ethanol evaporation during in-use of multidose containers. The formulation of the present invention comprises ethanol at concentration of less than about 40% w/v, preferably less than about 35% w/v, preferably less than about 30% w/v, preferably less than about 25% w/v, preferably less than about 20% w/v, preferably less than about 15% w/v, preferably less than about 12.5% w/v, preferably less than about 10% w/v, preferably less than about 5% w/v, preferably less than about 2% w/v, preferably about less than about 1% w/v, preferably less than about 0.5% w/v. In most preferred embodiments, the formulation of the present invention is essentially free from ethanol, comprising ethanol at concentration of less than about 10% w/v, preferably less than about 5% w/v, more preferably less than about 2% w/v, and most preferably less than about 0.5% w/v.

In some embodiments of the present invention, while ethanol may be present in trace amount as residual solvents in ingredients of the formulation, ethanol is not added intentionally as a solubilizer and its contribution to solubilize the drug is less than about 50%, preferably less than about 40%, preferably less than about 30%, preferably less than 20%, preferably less than about 10%, most preferably less than about 5%. In some embodiments of the present invention, ethanol is not a predominant solvent of the formulation of the present invention.

In some embodiments of the present invention, the stable liquid pharmaceutical composition of edaravone comprises one or more stabilizing ingredient to prevent degradation (e.g. oxidative degradation) of the active ingredient within the vehicle. In some embodiments of the present invention, the formulation comprises one or more antioxidants selected from the group comprising of sodium sulfite, potassium sulfite, calcium sulfite, sodium bisulfite, potassium bisulfite, ammonium bisulfite, sodium metabisulfite, potassium metabisulfite. In yet other embodiments of the present invention, the formulation comprises stabilizer selected from group comprising of L-cysteine, DL-cysteine, N-acetylcysteine, or their hydrochloride salts (such as L-cysteine hydrochloride monohydrate).

In some embodiments of the present invention, the stable liquid pharmaceutical formulation of edaravone comprises a chelating agent and/or a pH adjusting agent to maintain the physicochemical stability of the solution.

In some embodiments of the present invention, the stable liquid pharmaceutical formulation of edaravone is stored under conditions that minimize oxygen exposure, such as under an inert gas overlay (e.g., nitrogen) in a sealed container.

The resulting formulation is a clear, single-phase solution that is substantially free of undissolved particulate matter, lipids, and surfactants.

Clinically, the present invention provides a ready-to-use, high-concentration liquid that allows for low administration volumes (e.g., approx. 2-20 mL, preferably 5-10 mL) and is stable at room temperature conditions for extended period of time. This formulation overcomes the limitations of prior art suspensions, which require physical resuspension, and prior art extemporaneous solutions, which are limited to dilute concentrations and short in-use stability periods.

The present invention relates to a stable liquid formulation of edaravone, methods for their administration, processes for their manufacturing, and use of these compositions for treating diseases treatable by edaravone.

In an embodiment of the present invention, the stable liquid pharmaceutical formulation of edaravone, which is solution suitable for oral administration, comprises edaravone and at least one or more pharmaceutically acceptable excipients selected from the group comprising vehicles, preservatives, sweeteners, flavors, pH adjusting agents, buffering agents, viscosity adjusting agents, antioxidants, tonicity adjusting agents, chelating agents, permeability enhancing agents, mucoadhesive agents, stabilizers, complexing agents, crystallization inhibitors, emulsifying agent, suspending agents, surfactants, anti-foaming agents, dispersing agents, wetting agents, film forming agents and combinations thereof.

In some embodiments, without wishing to be bound by any theory, the edaravone solution oral formulations of the present invention are designed to provide a more rapid absorption and higher bioavailability thereby providing a significant clinical benefit to patients by overcoming the documented limitations of the oral suspension, and oral solid dosage forms (e.g. tablet or capsule) that require disintegration and/or dissolution.

In some other embodiments, without wishing to be bound by any theory, the edaravone solution oral formulations of the present invention are designed to be bioequivalent to currently approved oral suspension Radicava® ORS suspension.

In some embodiments of the present invention, the edaravone solution oral formulations of the present invention have no food effect or less food effect compared to Radicava® ORS suspension.

Another aspect relates to methods of treatment for amyotrophic lateral sclerosis (ALS), Acute Ischemic Stroke (AIS), Alzheimer's Disease (AD), Autism Spectrum Disorder (ASD), Acute Mountain Sickness (AMS), Hearing Loss/Vestibular Disorders, Contrast-Induced Acute Kidney Injury (CI-AKI), Parkinson's Disease (PD), and Traumatic Brain Injury (TBI) by administering an therapeutic effective amount of stable liquid pharmaceutical formulation of edaravone solution at concentration from about 5 mg/mL to about 50 mg/mL, from about 5 mg/mL to about 40 mg/mL, from about 10 mg/mL to 30 mg/mL, from about 15 mg/mL to about 25 mg/mL to the patients in need thereof.

### BRIEF DESCRIPTION OF DRAWINGS

FIG. 1 is a line graph showing the equilibrium solubility of edaravone in various solvents and solvent mixtures. The solubility of edaravone increases significantly when organic solvent concentrations are higher than 40% v/v in water.

FIG. 2 is a line graph showing the equilibrium solubility of edaravone in PEG 400:Water and PG:Water at various ratio with and without citric acid monohydrate. There is no significant change in solubility of edaravone in presence of 1% citric acid monohydrate.

FIG. 3 is a line graph showing equilibrium solubility of edaravone in PEG 4000/water mixture from about 10% w/v to about 60% w/v.

FIG. 4 is a bar graph showing equilibrium solubility of edaravone in different ratio of PG, PEG 4000, and water (% v/w/v).

FIG. 5 describes the bar graph for edaravone degradation in ethanol, PG, PEG 200, PEG 300, and PEG 400 when heated at 121° C./20 minutes.

FIG. 6 describes the line graph of edaravone degradation in different solvent ratio with water after heat treatment at 121° C. for 20 minutes.

FIG. 7 describes the line graph of edaravone in PG/water, PEG 400/water, and ethanol/water at 70:30 ratio with increasing amount of sodium metabisulfite after heat treatment of 121° C./20 minutes.

FIG. 8 describes the line graph of edaravone in PEG 200, PEG 300, and PEG 400 in water at 70% v/v with increasing amount of sodium metabisulfite after heat treatment of 121° C./20 minutes.

FIG. 9 describes a process flow diagram for method of preparing liquid formulation of edaravone by dissolving antioxidant in water, adding antioxidant aqueous solution into organic solvent, adding and dissolving edaravone, followed by filling and sealing with nitrogen overlay.

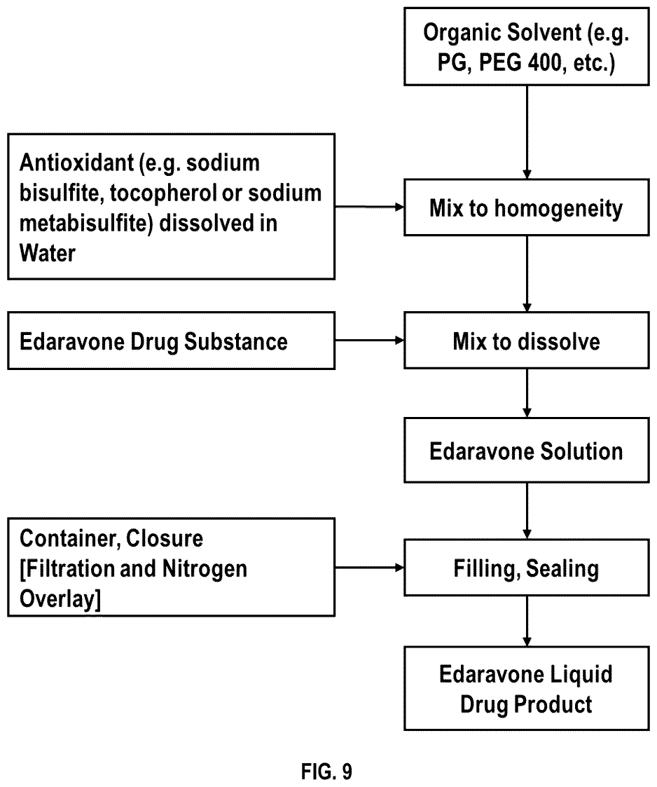

FIG. 10 describes a process flow diagram for method of preparing liquid formulation of edaravone by adding antioxidant/stabilizer solution (antioxidant/stabilizer dissolved in water) in organic solvent, followed by dissolving edaravone, preservative and flavor. The water and/or organic solvent is added to make the batch size, followed by filtration, filling and sealing with nitrogen overlay.

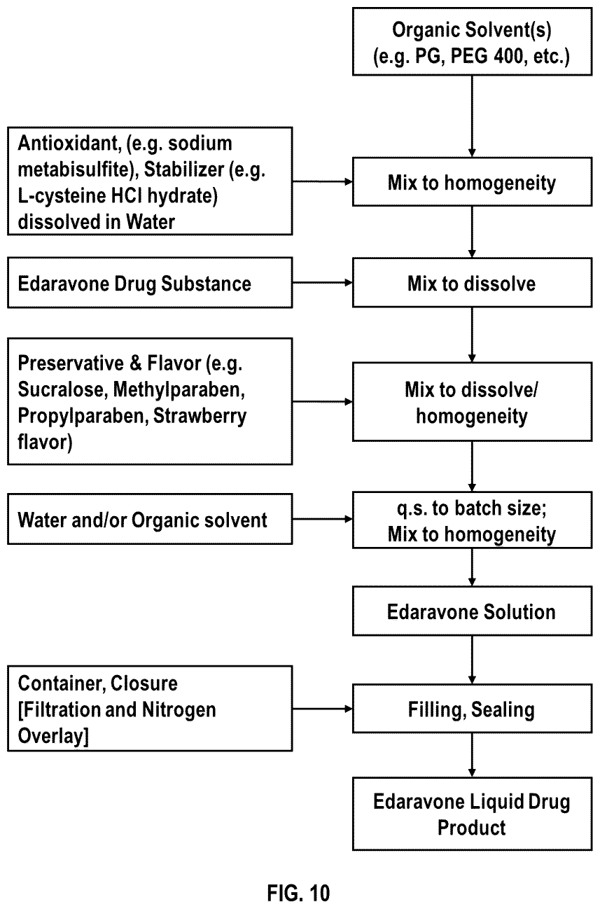

FIG. 11 describes a process flow diagram for method of preparing liquid formulation of edaravone by adding aqueous solution containing inactive ingredients in organic solvent, followed by dissolving edaravone, optionally adjusting pH between about 3 and about 5, and making up the volume to batch size using water and/or organic solvent. The bulk solution is then filtered, filled and sealed with nitrogen overlay.

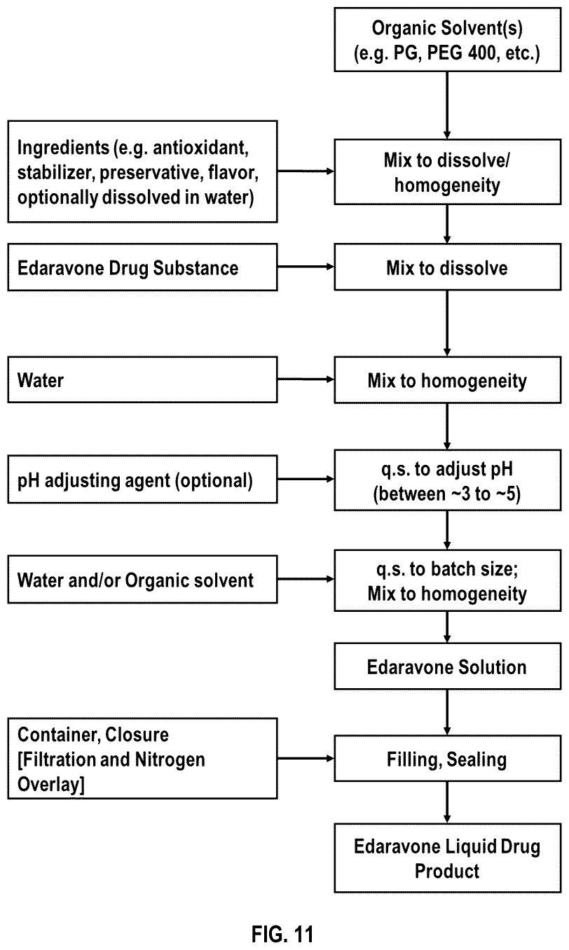

FIG. 12 describes two-step process of preparing liquid formulation of edaravone. In first step, citrate buffer is prepared using citric acid, sodium hydroxide and other water-soluble ingredients. In second step, edaravone is dissolved in organic solvents, followed by adding and mixing citrate buffer solution prepared in previous step, making up the volume to batch size, followed by filtration, filling, and sealing with nitrogen overlay.

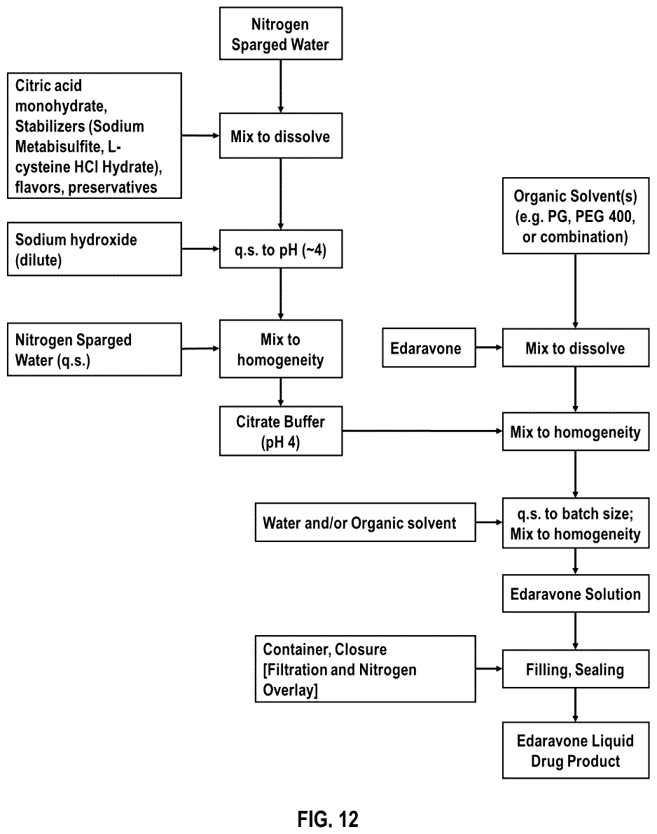

### DETAILED DESCRIPTION OF THE INVENTION

### Abbreviations

M: Month or Months, used in stability test results as timepoints such as 1M, 2M, 3M, etc.
W: Week or Weeks, used in stability test results as timepoints such as 1 W or 2 W.
RT: Room Temperature (about 20° C.), used in stability test results as storage conditions, such as RT/1M that indicates when sample stored at room temperature for about 1 month.
h: hour or hours used as unit of time such as Tₘₐₓ.
PG: Propylene Glycol
PEG: Polyethylene Glycol (such as PEG 200, PEG 300, PEG 400, PEG 4000)
SMBS: Sodium metabisulfite.

### Definitions

As used herein, the terms “edaravone” refers to edaravone free moiety or its pharmaceutically acceptable salts, solvates, or hydrates thereof. In principle, any crystalline or amorphous form of edaravone may be used to manufacture inventive pharmaceutical compositions of the present invention. It is understood that if the active ingredient or a portion thereof is provided as a salt, the concentration of active ingredient salt may be higher than the concentration of active ingredient.

As used herein, the terms “about” or “approximately” or symbol “~” refer to within a range of ±20%, optionally within ±10%, optionally within ±5%, optionally within ±3%, optionally within ±2%, optionally within ±1%, optionally within ±0.5%, optionally within ±0.1%, or optionally within ±0.01% of the stated value, except where such number would be less than 0% or exceed 100% of a possible value. As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “composition” or “formulation” may be used interchangeably and refer to the liquid pharmaceutical compositions of the present invention in the form of solution. The liquid formulation of the present invention may be delivered in the form of dosage form suitable for immediate release or modified release such as sustained release or delayed release.

The term “purity” describes the proportion of edaravone original form, expressed as a percentage (%). Purity is determined by calculating the area percentage of the edaravone peak (main peak or parent peak) when analyzed using a chromatographic method (HPLC) using UV detection (e.g., using a C18 column with 5 μm particles and 250×4.6 mm dimensions, detection at about 240 nm wavelength, and using gradient elution of an aqueous buffer at about pH 5.8 and an organic phase of acetonitrile and methanol, or a comparable method reported in the literature such as Baghel et al., 2018).

The terms “impurity” or “degradation impurity” or “edaravone-related impurity” used herein refer to any chemical compound that is undesired and generated from the degradation of edaravone during the formulation process or storage of the formulation and separated from Edaravone during chromatographic analysis (HPLC with UV detection). The concentrations of these compounds are reported as a percentage (%), representing the area percentage of the impurity peak relative to the edaravone peak. Such impurities may be reported individually or in total. The term “highest single impurity” or “maximum single impurity” or “maximum individual impurity” or “highest individual impurity” refers to any individual impurity with the highest percent peak area. The term “total impurities” refers to the combined amount of all impurities separated from edaravone peak and concentration of total impurities is reported in percentage (%) area. An alternative method for determining total impurities is to subtract the percentage of purity from 100.

The term “room temperature” or “RT” or “ambient temperature” as used herein refers to a temperature in a range of from about 15° C. to about 25° C. (e.g., about 59° F. to about 77° F.). It is understood that degradation rates are temperature-dependent, and “stability at room temperature” generally refers to stability observed under typical ambient laboratory conditions (e.g., approximately 20° C. to 25° C.). Unless otherwise specified, experimental data reported at “room temperature” refers to storage under ambient, unregulated relative humidity (RH) conditions (typically ranging from about 30% to about 60% RH).

The term “seal” or “sealing” used herein refers to closing the primary packaging container with suitable closure by means of airtight sealing, such as induction sealing or aluminum crimping, that prevent the air/oxygen ingress into the container to obtain the stable product.

The term “stable” used herein refers to the invented composition that has less 10%, preferably less than 5%, preferably less than 3%, preferably less than 2% of the total degradation impurities after extended period at room temperature (15-25° C.), or at refrigerated condition, i.e. 2-8° C. In some embodiments, the invented composition that has less than 10%, preferably less than 5%, preferably less than 3%, preferably less than 2%, preferably less than 1% of the total degradation impurities after shorter period at accelerated conditions such as about 40° C., about 60° C., about 70° C., or about 120° C. (such as autoclave for about 5 to 20 minutes) may also be considered stable.

As used herein, the term “essentially free from visible particles” refers to a liquid formulation that appears clear and homogenous to the unaided eye when viewed under standard ambient lighting conditions. This term implies that the formulation is not suspension and does not contain sediments, crystals or precipitates that would require resuspension prior to administration but does not exclude the presence of trace or negligible particulate matter that typically does not materially affect the dosing uniformity of the liquid formulation.

As used herein, the term “shelf life” refers to the length of time that a formulation may be stored without becoming unfit for medical use. Examples of compositions which are unfit for medical use include, but are not limited to, compositions with unacceptably high impurity levels (such as more than 10% total impurities or less than 90% purity), and/or the presence of physical changes such as precipitation.

As used herein, the term “step” is not particularly limiting. For example, each step as described herein may be a discrete step such that steps are performed sequentially upon completion of a preceding step. Alternatively, at least a portion of the method may be continuous, or the steps may be performed out of the order stated.

It should be noted that the unit of measurement for the quantity of any ingredients in this invention, such as “%”, typically represents % w/w. In certain embodiments, this quantity may also be expressed using the unit % w/v. If the units are presented in % w/w in the present disclosure, it is also considered as % w/v, if the difference in concentration resulting from the conversion is less than about 10%. It is also understood that units presented in “mg/mL” or “mg per mL” can be converted into “% w/v” using this formula: % w/v=[mg/mL]×0.1; or mg/mL=[% w/v]×10.

As used herein, the term “parenteral”, refers to the route of administration of the formulation of the present invention other than topical or oral. Examples of parenteral routes include intramuscular, intravascular (including intraarterial or intravenous), intraorbital, retrobulbar, intranasal, intrathecal, intraventricular, intraspinal, intraperitoneal, intrapulmonary, intracisternal, intracapsular, intrasternal, peribulbar, or intralesional administration.

As used herein, the terms “micron”, or “microns” or “micrometer” or “μm” refer to one millionth of a meter. Similarly, “μg” refers to one millionth of a gram.

As used herein, the term “liquid carrier” refers to a liquid medium comprising one or more pharmaceutically acceptable excipients or inactive ingredients described in this disclosure.

The term “stabilizing amount” refers to the concentration of an ingredient (e.g. antioxidant, or stabilizer) that enables the formulation of a stable liquid containing edaravone.

The term “nitrogen overlay” refers to purging nitrogen in head space of liquid filled container before closing and sealing the container. The nitrogen or other inert gas such as helium or argon is purged to replace headspace oxygen and reduce oxygen concentration in headspace less than atmospheric concentration (i.e. about 21%). In an embodiment of the present invention, nitrogen overlay may not be required if the liquid is filled to about the full capacity of the container. In another embodiment, nitrogen is required to reduce the headspace oxygen concentration to less than about 20%, more preferably less than about 15%, more preferably less than about 10%, more preferably less than 5%, and most preferably less than 2%.

As used herein, the term “essentially free from” a specified component means that the component is either not intentionally added to the formulation, or, if present, is present at a level that does not materially affect the stability or performance characteristics of the claimed formulation. For example, a formulation essentially free from the component would exhibit stability comparable (or insignificantly different) to an identical formulation prepared without such component.

As used herein, the terms “subject” or “patient” refer to a mammal, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), but are not so limited. Subjects include human healthy volunteers and/or patients suffering from one or more diseases treatable and/or preventable by edaravone. The human subject or patient may be a pediatric, adult, or a geriatric subject. The human subject may be of either sex.

The present invention relates to a stable liquid pharmaceutical formulation comprising: (a) edaravone; (b) one or more pharmaceutically acceptable excipients selected from the group comprising vehicles, preservatives, sweeteners, flavors, pH adjusting agents, buffering agents, viscosity adjusting agents, antioxidants, tonicity adjusting agents, chelating agents, permeability enhancing agents, mucoadhesive agents, stabilizers, complexing agents, crystallization inhibitors, emulsifying agent, suspending agents, surfactants, anti-foaming agents, dispersing agents, wetting agents, film forming agent, and combinations thereof; and wherein upon storage for at least three months at room temperature, the formulation has: (i) at least about 95% pure edaravone, (ii) less than about 5% of total degradant impurities.

In an embodiment, the invention provides stable liquid pharmaceutical compositions of edaravone, wherein edaravone is present at a concentration in the range from about 5 mg/mL to about 100 mg/mL, optionally from about 10 mg/mL to about 50 mg/mL, optionally from about 15 mg/mL to about 25 mg/mL.

In some embodiments, the present invention provides liquid pharmaceutical formulations, wherein the concentration of edaravone is about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/mL, about 60 mg/mL, about 61 mg/mL, about 62 mg/mL, about 63 mg/mL, about 64 mg/mL, about 65 mg/mL, about 66 mg/mL, about 67 mg/mL, about 68 mg/mL, about 69 mg/mL, about 70 mg/mL, about 71 mg/mL, about 72 mg/mL, about 73 mg/mL, about 74 mg/mL, about 75 mg/mL, about 76 mg/mL, about 77 mg/mL, about 78 mg/mL, about 79 mg/mL, about 80 mg/mL, about 81 mg/mL, about 82 mg/mL, about 83 mg/mL, about 84 mg/mL, about 85 mg/mL, about 86 mg/mL, about 87 mg/mL, about 88 mg/mL, about 89 mg/mL, about 90 mg/mL, about 91 mg/mL, about 92 mg/mL, about 93 mg/mL, about 94 mg/mL, about 95 mg/mL, about 96 mg/mL, about 97 mg/mL, about 98 mg/mL, about 99 mg/mL, about 100 mg/mL, about 101 mg/mL, about 102 mg/mL, about 103 mg/mL, about 104 mg/mL, about 105 mg/mL. The concentration of edaravone may be from about 5 mg/mL to about 50 mg/mL, preferably from about 10 mg/mL to about 30 mg/mL, preferably from about 15 mg/mL to about 25 mg/mL.

In an embodiment of the present invention, the liquid pharmaceutical formulation of edaravone may be provided as solution, suspension, or emulsion, preferably as a solution or emulsion, more preferably as a solution.

In one embodiment, the formulation is suitable for administration selected from oral, sublingual, buccal or intranasal routes.

In another embodiment, the formulation is sterile and suitable for parenteral or injectable administration.

In an embodiment of the present invention, the stable liquid pharmaceutical formulation of edaravone is provided in the form of solution dosage form.

### Oral Formulations

In an embodiment of the present invention, the stable liquid pharmaceutical formulation of edaravone is provided in a dosage form suitable for oral administration.

In an embodiment of the present invention, the stable liquid pharmaceutical formulation comprises edaravone at concentration from about 10 mg/mL to about 50 mg/mL, preferably from about 15 mg/mL to about 25 mg/mL.

In an embodiment of the present invention, the stable liquid pharmaceutical formulation comprises edaravone at concentration of about 21 mg/mL.

In an embodiment, the present invention provides a stable liquid pharmaceutical formulation suitable for oral administration, wherein the formulation comprises (i) edaravone at a concentration in the range from about 5 mg/mL to about 50 mg/mL, and (ii) at least one pharmaceutically acceptable vehicle.

In an embodiment, the present invention provides a liquid pharmaceutical formulation suitable for oral administration, wherein the formulation comprises (i) edaravone at a concentration in the range from about 5 mg/mL to about 50 mg/mL; (ii) at least one pharmaceutically acceptable excipient; and (iii) at least one pharmaceutically acceptable vehicle.

In an embodiment, the present invention provides a liquid pharmaceutical formulation suitable for oral administration, wherein the formulation comprises (i) edaravone at a concentration in the range from about 5 mg/mL to about 50 mg/mL; (ii) at least one pharmaceutically acceptable excipients selected from the group comprising preservatives, sweeteners, flavors, pH adjusting agents, buffering agents, viscosity adjusting agents, antioxidants, tonicity adjusting agents, chelating agents, permeability enhancing agents, mucoadhesive agents, stabilizers, complexing agents, crystallization inhibitors, emulsifying agent, suspending agents, surfactants, anti-foaming agents, dispersing agents, wetting agents, film forming agents and combinations thereof; and (iii) at least one pharmaceutically acceptable vehicle selected from propylene glycol, polyethylene glycol, water, or combination thereof.

In an embodiment of the present invention, the liquid formulation of edaravone suitable for oral administration is solution comprising edaravone (from about 1% w/v to about 3% w/v, preferably about 2% w/v), vehicle (q.s. to 100% w/v) and one or more optional ingredients selected from group comprising of sweetener (from about 0.1% w/v to about 70% w/v, preferably from about 0.1% w/v to about 5% w/v), pH adjusting agent (q.s. to adjust pH between about 2 and about 7, optionally between about 2 and about 5), stabilizer (from about 0.001% w/v to about 1% w/v, preferably from about 0.01% w/v to about 0.1% w/v), surfactant (from about 0.01% w/v to about 1% w/v, preferably from about 0.01% w/v to about 0.2% w/v), preservative (from about 0.01% w/v to about 4% w/v), antioxidant (from about 0.001% w/v to about 1% w/v), viscosity adjusting agent (from about 0.01% w/v to about 20% w/v, preferably from about 0.1% w/v to about 5% w/v), and flavoring agent (from about 0.001% w/v to about 2% w/v).

In an embodiment of the present invention, the liquid formulation of edaravone suitable for oral administration is solution comprising edaravone (from about 1% w/v to about 3% w/v), vehicle (q.s. to 100% w/v) and one or more optional ingredient selected from group comprising of sweetener (from about 0.1% w/v to about 5% w/v), pH adjusting agent (q.s. to adjust pH between about 2 and about 5), stabilizer (from about 0.001% w/v to about 0.1% w/v), viscosity adjusting agent (from about 0.1% w/v to about 5% w/v), preservative (from about 0.01% w/v to about 1% w/v), antioxidant (from about 0.001% w/v to about 0.2% w/v), and flavoring agent (from about 0.01% w/v to about 1% w/v).

In an embodiment of the present invention, the liquid formulation of edaravone suitable for oral administration is solution comprising edaravone (from about 1% w/v to about 3% w/v, preferably about 2% w/v), sucralose (from about 0.1% w/v to about 5% w/v), methyl paraben (from about 0.01% w/v to about 2% w/v), propylparaben (from about 0.001% to about 0.2% w/v), sodium metabisulfite (from about 0.01% w/v to about 0.2% w/v), L-cysteine (from about 0.01% to about 0.1% w/v), flavoring agent (from about 0.01% w/v to about 1% w/v), propylene glycol or polyethylene glycol or combination (from about 50% v/v to about 95% v/v, preferably from about 70% v/v to about 90% v/v), and purified water (q.s. to 100% v/v).

In an embodiment, the present invention provides a liquid pharmaceutical formulation of edaravone suitable for oral administration, wherein the formulation contains a preservative and intended for unit dose or multiple dose administration, preferably multiple dose administration.

### Intranasal Formulations

In an embodiment of the present invention, the stable liquid pharmaceutical formulation of edaravone is provided in a dosage form, such as solution, emulsion or suspension, preferably solution, suitable for intranasal administration.

In an embodiment, the present invention provides a liquid pharmaceutical formulation suitable for intranasal administration, wherein the composition comprises (i) edaravone at a concentration in the range from about 5 mg/mL to about 100 mg/mL, optionally from about 10 mg/mL to about 70 mg/mL; and (ii) at least one pharmaceutically acceptable vehicle.

In an embodiment, the present invention provides a liquid pharmaceutical formulation suitable for intranasal administration, wherein the composition comprises (i) edaravone at a concentration in the range from about 5 mg/mL to about 100 mg/mL, optionally from about 10 mg/mL to about 70 mg/mL; (ii) at least one pharmaceutically acceptable excipient; and (iii) at least one pharmaceutically acceptable vehicle.

In an embodiment, the present invention provides a liquid pharmaceutical formulation suitable for intranasal administration, wherein the composition comprises (i) edaravone at a concentration in the range from about 5 mg/mL to about 100 mg/mL, optionally from about 10 mg/mL to about 70 mg/mL; (ii) at least one pharmaceutically acceptable excipient selected from group comprising of preservatives, sweeteners, flavors, pH adjusting agents, buffering agents, viscosity adjusting agents, antioxidants, tonicity adjusting agents, chelating agents, permeation enhancers, mucoadhesive agents, stabilizers, complexing agents, crystallization inhibitors, emulsifying agents, suspending agents, surfactants, anti-foaming agents, dispersing agents, and wetting agents; and (iii) at least one pharmaceutically acceptable vehicle.

In an embodiment, the present invention provides a liquid pharmaceutical formulation suitable for intranasal administration, wherein the composition comprises (i) edaravone at a concentration in the range from about 10 mg/mL to about 70 mg/mL; (ii) at least one pharmaceutically acceptable excipient selected from group comprising of preservatives, sweeteners, flavors, pH adjusting agents, buffering agents, viscosity adjusting agents, antioxidants, tonicity adjusting agents, chelating agents, permeation enhancers, mucoadhesive agents, stabilizers, complexing agents, crystallization inhibitors, emulsifying agents, suspending agents, surfactants, anti-foaming agents, dispersing agents, and wetting agents; and (iii) at least one pharmaceutically acceptable vehicle selected from group comprising of propylene glycol, polyethylene glycol (PEG, such as PEG 400, PEG 4000, etc.), water, or combination thereof.

In another embodiment of the present invention, edaravone may be present at a concentration in the range from about 10 mg/mL to about 70 mg/mL, optionally from about 25 mg/mL to about 50 mg/mL.

In an embodiment, the present invention provides a liquid pharmaceutical formulation of edaravone suitable for intranasal administration, wherein the formulation is sterile, preservative free intended for unit dose or bi-dose or multiple dose administration.

In an embodiment, the present invention provides a liquid pharmaceutical formulation of edaravone suitable for intranasal administration, wherein the formulation comprises a preservative and intended for unit dose or bi-dose or multiple dose administration, preferably multiple dose administration.

In an embodiment of the present invention, the liquid formulation of edaravone suitable for intranasal administration comprises edaravone (from about 1% w/v to about 10% w/v, preferably from about 2% w/v to about 7% w/v), vehicle (q.s. to 100% w/v) and one or more optional ingredients selected from group comprising of preservative (from about 0.001% w/v to about 0.5% w/v), chelating agent (from about 0.001% w/v to about 1% w/v), pH adjusting agent (q.s. to adjust pH between about 2 and about 6, optionally between about 3 and about 5), surfactant (from about 0.01% w/v to about 1% w/v, preferably from about 0.01% w/v to about 0.2% w/v), permeability enhancing agent (from about 0.01% w/v to about 1% w/v), viscosity adjusting agent (from about 0.001% w/v to about 1% w/v, preferably from about 0.01% w/v to about 0.5% w/v), stabilizer (from about 0.001% w/v to about 1% w/v, preferably from about 0.01% w/v to about 0.5% w/v), antioxidant (from about 0.001% w/v to about 0.5% w/v), and tonicity adjusting agent (from about 0.1% w/v to about 5% w/v).

In an embodiment of the present invention, the liquid formulation of edaravone suitable for intranasal administration comprises edaravone (from about 2% w/v to about 7% w/v), vehicle (q.s. to 100% w/v) and one or more optional ingredients selected from group comprising of preservative (from about 0.001% w/v to about 0.5% w/v), chelating agent (from about 0.001% w/v to about 1% w/v), pH adjusting agent (q.s. to adjust between about 3 and about 5), surfactant (from about 0.01% w/v to about 0.2% w/v), permeability enhancing agent (from about 0.01% w/v to about 1% w/v), viscosity adjusting agent (preferably from about 0.01% w/v to about 0.5% w/v), stabilizer (from about 0.01% w/v to about 0.5% w/v), antioxidant (from about 0.001% w/v to about 0.5% w/v), and tonicity adjusting agent (from about 0.1% w/v to about 5% w/v).

In an embodiment of the present invention, the liquid formulation of edaravone suitable for intranasal administration comprises edaravone (from about 2% w/v to about 7% w/v), vehicle (q.s. to 100% w/v) and one or more optional ingredients selected from group comprising of preservative (such as benzalkonium chloride, from about 0.001% w/v to about 0.5% w/v), chelating agent (such as disodium edetate, from about 0.001% w/v to about 1% w/v), pH adjusting agent (such as citric acid and/or sodium hydroxide, q.s. to adjust between about 3 and about 5), surfactant (such as polysorbate 80, from about 0.01% w/v to about 0.2% w/v), permeability enhancing agent (such as n-dodecyl beta-D-maltoside, from about 0.01% w/v to about 1% w/v), viscosity adjusting agent (such as hypromellose or hydroxypropyl cellulose, preferably from about 0.01% w/v to about 0.5% w/v), stabilizer (such as L-cysteine hydrochloride, from about 0.01% w/v to about 0.5% w/v), antioxidant (such as sodium bisulfite or sodium metabisulfite, from about 0.001% w/v to about 0.5% w/v), and tonicity adjusting agent (from about 0.1% w/v to about 5% w/v).

### Parenteral Formulations

In an embodiment of the present invention, the liquid pharmaceutical formulation of edaravone is provided in a dosage form, such as solution, emulsion or suspension, preferably solution, suitable for parenteral administration.

In an embodiment, the present invention provides a liquid pharmaceutical formulation suitable for parenteral administration, wherein the composition comprises (i) edaravone at a concentration in the range from about 5 mg/mL to about 100 mg/mL, optionally from about 10 mg/mL to about 50 mg/mL; and (ii) at least one pharmaceutically acceptable vehicle.

In an embodiment, the present invention provides a liquid pharmaceutical formulation suitable for parenteral administration, wherein the composition comprises (i) edaravone at a concentration in the range from about 5 mg/mL to about 100 mg/mL, optionally from about 10 mg/mL to about 50 mg/mL; (ii) at least one pharmaceutically acceptable excipient; and (iii) at least one pharmaceutically acceptable vehicle.

In an embodiment, the present invention provides a liquid pharmaceutical formulation suitable for parenteral administration, wherein the composition comprises (i) edaravone at a concentration in the range from about 5 mg/mL to about 100 mg/mL, optionally from about 10 mg/mL to about 50 mg/mL; (ii) at least one pharmaceutically acceptable excipient selected from group comprising of preservatives, pH adjusting agents, buffering agents, viscosity adjusting agents, antioxidants, tonicity adjusting agents, chelating agents, stabilizers, complexing agents, crystallization inhibitors, emulsifying agents, suspending agents, surfactants, anti-foaming agents, dispersing agents, and wetting agents; and (iii) at least one pharmaceutically acceptable vehicle.

In an embodiment, the present invention provides a liquid pharmaceutical formulation suitable for parenteral administration, wherein the composition comprises (i) edaravone at a concentration in the range from about 10 mg/mL to about 50 mg/mL; (ii) at least one pharmaceutically acceptable excipient selected from group comprising of preservatives, pH adjusting agents, buffering agents, viscosity adjusting agents, antioxidants, tonicity adjusting agents, chelating agents, stabilizers, complexing agents, crystallization inhibitors, emulsifying agents, suspending agents, surfactants, anti-foaming agents, dispersing agents, and wetting agents; and (iii) at least one pharmaceutically acceptable vehicle selected from group comprising of propylene glycol, polyethylene glycol (PEG, such as PEG 400, PEG 4000, etc.), water, or combination thereof.

In another embodiment of the present invention, edaravone may be present at a concentration in the range from about 10 mg/mL to about 70 mg/mL, optionally from about 25 mg/mL to about 50 mg/mL.

In an embodiment, the present invention provides a liquid pharmaceutical formulation of edaravone suitable for parenteral administration, wherein the formulation is sterile, preservative free intended for single dose administration.

In an embodiment, the present invention provides a liquid pharmaceutical formulation of edaravone suitable for parenteral administration, wherein the formulation comprises a preservative and intended for multiple dose administration.

In an embodiment of the present invention, the liquid formulation of edaravone suitable for parenteral administration comprises edaravone (from about 1% w/v to about 10% w/v, preferably from about 2% w/v to about 7% w/v), vehicle (q.s. to 100% w/v) and one or more optional ingredients selected from group comprising of preservative (from about 0.001% w/v to about 0.5% w/v), chelating agent (from about 0.001% w/v to about 1% w/v), pH adjusting agent (q.s. to adjust pH between about 2 and about 6, optionally between about 3 and about 5), surfactant (from about 0.01% w/v to about 1% w/v, preferably from about 0.01% w/v to about 0.2% w/v), viscosity adjusting agent (from about 0.001% w/v to about 1% w/v, preferably from about 0.01% w/v to about 0.5% w/v), stabilizer (from about 0.001% w/v to about 1% w/v, preferably from about 0.01% w/v to about 0.5% w/v), antioxidant (from about 0.001% w/v to about 0.5% w/v), and tonicity adjusting agent (from about 0.1% w/v to about 5% w/v).

In an embodiment of the present invention, the liquid formulation of edaravone suitable for parenteral administration comprises edaravone (from about 2% w/v to about 7% w/v), vehicle (q.s. to 100% w/v) and one or more optional ingredients selected from group comprising of preservative (from about 0.001% w/v to about 0.5% w/v), chelating agent (from about 0.001% w/v to about 1% w/v), pH adjusting agent (q.s. to adjust between about 3 and about 7), surfactant (from about 0.01% w/v to about 0.2% w/v), viscosity adjusting agent (preferably from about 0.01% w/v to about 0.5% w/v), stabilizer (from about 0.010% w/v to about 0.50% w/v), antioxidant (from about 0.0010% w/v to about 0.50% w/v), and tonicity adjusting agent (from about 0.1% w/v to about 5% w/v).

In an embodiment of the present invention, the liquid formulation of edaravone suitable for parenteral administration comprises edaravone (from about 2% w/v to about 7% w/v), vehicle (q.s. to 100% w/v) and one or more optional ingredients selected from group comprising of preservative (such as benzalkonium chloride, from about 0.001% w/v to about 0.5% w/v), chelating agent (such as disodium edetate, from about 0.001% w/v to about 1% w/v), pH adjusting agent (such as citric acid and/or sodium hydroxide, q.s. to adjust between about 3 and about 6), surfactant (such as polysorbate 80, from about 0.01% w/v to about 0.2% w/v), viscosity adjusting agent (such as hypromellose or hydroxypropyl cellulose, preferably from about 0.01% w/v to about 0.5% w/v), stabilizer (such as L-cysteine hydrochloride, from about 0.01% w/v to about 0.5% w/v), antioxidant (such as sodium bisulfite or sodium metabisulfite, from about 0.001% w/v to about 0.5% w/v), and tonicity adjusting agent (from about 0.1% w/v to about 5% w/v).

In one embodiment of the invention, the formulation is an oil-based liquid depot. This formulation comprises edaravone dissolved in a biocompatible, and biodegradable oil vehicle suitable for parenteral injection. Upon intramuscular or subcutaneous administration, this composition forms a localized depot from which the edaravone slowly partitions into the systemic circulation. The release of the drug is thus sustained over a prolonged duration. Suitable oils for such formulations are not limited but may include sesame oil, cottonseed oil, castor oil, soybean oil, ethyl oleate, benzyl benzoate, and medium-chain triglycerides. To enhance stability and optimize performance, the oil-based formulation may further comprise one or more pharmaceutically acceptable excipients. These can include antioxidants, such as alpha-tocopherol, as well as viscosity-modifying agents or co-solvents to improve the solubility and stability of the active ingredient within the vehicle.

In some embodiments of the present invention, the liquid formulation of edaravone may be encapsulated in soft gelatin capsule for oral delivery for the said advantages of the liquid formulations.

In yet another embodiment, the present invention provides a liquid pharmaceutical formulation of edaravone suitable for parenteral route of administration, wherein the formulation is sterilized by sterile filtration without use of heat. In an embodiment, the present invention provides a liquid pharmaceutical formulation of edaravone suitable for parenteral route of administration, wherein the formulation is ready-to-dilute without requiring reconstitution at time of use prior to administration.

In another embodiment, the present invention provides a liquid pharmaceutical formulation of edaravone suitable for parenteral route of administration, wherein the formulation is ready-to-use without requiring dilution at the time of administration prior to use.

### Liquid Formulation Filled Solid Dosage Forms

In certain embodiments, the stable liquid formulations of edaravone described herein are intended for oral administration encapsulated within either a liquid-filled soft gelatin capsule or a liquid-filled hard gelatin capsule. These dosage forms leverage the stability of the liquid formulation while providing alternative delivery options.

In some embodiments, the capsules are provided as immediate release liquid-filled solid formulations, wherein the liquid fill is released rapidly upon disintegration of the capsule shell in the stomach. In some other embodiments, the capsules are provided as delayed-release liquid-filled solid formulations by encapsulating the liquid formulation in standard soft or hard gelatin capsules followed by the application of an enteric (delayed-release) coating to the capsule exterior. In yet other embodiments of the present invention, specialized hard capsule shells composed of materials suitable for delayed release (e.g., DRCaps® made from HPMC, ACGcaps™ HX, or similar technologies) are used, or specialized soft gelatin shell compositions providing enteric properties are used. A key advantage of these delayed-release liquid-filled dosage forms is the potential for more rapid drug release and availability for absorption once the enteric coating dissolves or the delayed-release shell opens in the intestine, compared to conventional delayed-release tablets, pellets or multiarticulate which require the solid core or particles to disintegrate and dissolve before absorption can occur. The liquid formulation ensures the edaravone is already in solution, potentially accelerating onset of absorption post-gastric transit.

The liquid formulation of the present invention is adaptable to a wide range of oral dosage forms by utilizing various commercially available capsule sizes, thereby enabling the creation of multiple dosage strengths suitable for diverse patient needs. In certain embodiments, the formulation is encapsulated in a liquid-filled hard gelatin capsule. The selection of hard gelatin capsule sizes may include, but is not limited to, standard sizes such as 5, 4, 3, 2, 1, 0, 00, or 000, which are capable of accommodating fill volumes ranging from approximately 0.13 mL to 1.37 mL. In alternative embodiments, the formulation is encapsulated in a soft gelatin capsule (softgel), which may be configured in various shapes, including round, oval, or oblong. For example, oval softgels may range from a size of 2 minims to 20 minims (accommodating approximately 0.12 mL to 1.23 mL), while oblong softgels may range from 4 minims to 22 minims (accommodating approximately 0.25 mL to 1.35 mL). This flexibility allows for precise dose titration and facilitates the development of formulations tailored for specific patient populations, such as pediatric or geriatric patients who benefit from smaller, easier-to-swallow capsules. Any of the aforementioned capsule sizes, whether hard or soft gelatin, may be further processed to achieve immediate or delayed release profiles, thus broadening the therapeutic utility of the edaravone liquid formulation.

### Pharmaceutical Excipients

In an embodiment of the present invention, the excipients in the formulation are selected from:
(a) Vehicles selected from the group comprising water (e.g. purified water for oral or intranasal dosage forms and water for injection for parenteral dosage forms), sorbitol, sucrose, sugar, mannitol, xylitol, propylene glycol, polyethylene glycol, alcohol, glycerin, lactose, cellulose, starch, triglycerides, oil, and combinations thereof,
(b) Preservatives selected from the group comprising benzoic acid and the sodium or potassium salts thereof, ethanol, isopropanol, methanol, butyl alcohol, benzalkonium chloride, benzyl alcohol, benzethonium chloride, butylparaben, cetylpyridinium chloride, chlorobutanol, chlorocresol, cresol, dehydroacetic acid, ethylparaben, ethylparaben sodium, methylparaben, methylparaben sodium, phenol, phenylethyl alcohol, phenylmercuric acetate, phenylmercuric nitrate, potassium benzoate, potassium sorbate, propylparaben, propylparaben sodium, sodium dehydroacetate, sodium propionate, sorbic acid, thimerosal, thymol, and combinations thereof,
(c) Sweeteners selected from the group comprising glucose, sucralose, neotame, aspartame, advantame, trehalose, fructose, xylose, dextrose, galactose, tagatose, maltose, sucrose, glycerol, dulcitol, mannitol, lactitol, sorbitol, xylitol, saccharin or the corresponding sodium, potassium, or calcium salts, cyclamate or its sodium or calcium salt, acesulfame or its potassium salt, dulcin, ammonium glycyrrhizinate, alitame, inulin, steviol glycoside, isomalt, neohesperidin dihydrochalcone, thaumatin, and combinations thereof,
(d) Flavoring agents selected from the group comprising vanilla, citrus oils including lemon, orange, grape, lime, and grapefruit, and fruit essences including apple, banana, pear, peach, strawberry, raspberry, cherry, plum, pineapple, apricot, peppermint, and tutti-frutti flavors, and combinations thereof;
(e) pH adjusting agents selected from the group comprising acetic acid, adipic acid, ammonia, ascorbic acid, barium hydroxide, benzoic acid, calcium hydroxide, carbonic acid, cesium hydroxide, citric acid, formic acid, fumaric acid, gluconic acid, glutamic acid, hydrochloric acid, isothionic acid, lactic acid, lithium hydroxide, malic acid, maleic acid, mandelic acid, methane sulfonic acid, monopotassium phosphate, monosodium phosphate, mucic acid, nitric acid, oxalic acid, p-toluenesulfonic acid, pantothenic acid, phosphoric acid, phthalic acid, potassium hydroxide, pyrophosphoric acid, rubidium hydroxide, sodium hydroxide, strontium hydroxide, succinic acid, sulfuric acid, tartaric acid, and combinations thereof;
(f) Buffering agents selected from the group comprising salts of citric acid, tartaric acid, ascorbic acid, phosphoric acid, acetic acid, carbonic acid, phthalic acid, succinic acid, glutamic acid, benzoic acid, gluconic acid, formic acid, fumaric acid, lactic acid, isothionic acid, malic acid, maleic acid, mandelic acid, nitric acid, mucic acid, oxalic acid, pantothenic acid, p-toluenesulfonic acid, methane sulfonic acid, sulfuric acid, hydrochloric acid, or combinations thereof, and optionally pharmaceutically acceptable amino acids such as methionine, cysteine, or glycine;
(g) Viscosity adjusting agents selected from the group comprising hydroxypropyl methylcellulose (HPMC), sodium carboxymethylcellulose, hydroxyethyl cellulose, methylcellulose, carbomers, polyvinylpyrrolidone (PVP), poloxamers, polyvinyl alcohol, xanthan gum, guar gum, acacia, tragacanth, and carrageenan, and combinations thereof;
(h) Antioxidants selected from the group comprising methionine, sodium bisulfite, potassium bisulfite, sodium metabisulfite, potassium metabisulfite, sodium ascorbate, ascorbic acid, butylated hydroxytoluene (BHT), butylated hydroxyanisole (BHA), cysteine, glutathione, monothioglycerol, propyl gallate, gallic acid, sodium or potassium sulfite, tocopherol or its salts and esters such as D-alpha-tocopheryl polyethylene glycol 1000 succinate, and combinations thereof;
(i) Tonicity adjusting agents selected from the group comprising alanine, arginine, betaine, boric acid, calcium chloride, calcium lactate, dextran, dextrose, fructose, glycerin, glycine, glucose, histidine, hydroxyethyl starch, lactose, lysine, magnesium chloride, magnesium sulfate, maltose, mannitol, polyethylene glycol, potassium chloride, potassium citrate, proline, propylene glycol, raffinose, ribose, sodium acetate, sodium benzoate, sodium bicarbonate, sodium citrate, sodium chloride, sodium gluconate, sodium lactate, sodium metabisulfite, sodium phosphate, sodium sulfate, sodium tartrate, sodium thiosulfate, sorbitol, sucrose, taurine, trehalose, tromethamine, xylitol, and mixtures thereof;
(j) Chelating agents selected from the group comprising edetate disodium (EDTA), edetate trisodium, edetate tetrasodium, edetate calcium disodium, diethylene triamine pentaacetate, or derivatives thereof;
(k) Permeability enhancing agents selected from the group comprising alcohols, fatty acids (including capric, caproic, lauric, myristic, oleic, palmitic, stearic acids), esters thereof, citric acid, chitosan, glycofurol, isopropyl myristate, sodium caprate, sodium glycocholate, sodium taurocholate, sodium lauryl sulfate, menthol, limonene, thymol, polysorbates, poloxamers, benzalkonium chloride, cetylpyridinium chloride, and combinations thereof;
(l) Mucoadhesive agents selected from the group comprising natural polymers such as chitosan, alginate, hyaluronic acid, gelatin, tragacanth, xanthan gum, guar gum, and pectin, and synthetic or semi-synthetic polymers such as carbomers (Carbopol), polyvinyl alcohol, polyvinylpyrrolidone, polyethylene oxide, polyacrylic acid, hydroxypropyl methylcellulose, methylcellulose, carboxymethyl cellulose, and poloxamers, and mixtures thereof;
(m) Stabilizers selected from amino acids such as alanine, glycine, glutamate, arginine, lysine, cysteine, or methionine; salts such as sodium chloride, sodium sulfate, potassium hydroxide, calcium hydroxide; organic acids such as lactic, succinic, pyruvic, citric, glycolic acid; metal ions such as zinc, magnesium, calcium; gums, cellulose derivatives, cyclodextrins, sugars, sugar alcohols, or polysaccharides;
(n) Complexing agents selected from the group comprising cyclodextrins including α-, β-, and γ-cyclodextrin and derivatives thereof (hydroxypropyl-β-cyclodextrin, sulfobutyl ether-β-cyclodextrin), ethylenediaminetetraacetic acid, citric acid, tartaric acid, or other inclusion or coordination complex formers;
(o) Crystallization inhibitors selected from the group comprising polyvinylpyrrolidone (PVP), polyethylene glycol, hydroxypropyl methylcellulose, hydroxypropyl cellulose, methylcellulose, sodium carboxymethylcellulose, polyvinyl alcohol, poloxamers, povidone, copovidone, cyclodextrins, and polysorbates;
(p) Suspending agents selected from the group comprising ethylcellulose, sodium carboxymethylcellulose, hydroxypropyl methylcellulose, polyvinylpyrrolidone, alginate, guar gum, xanthan gum, carrageenan, acacia gum, tragacanth, chitosan, dextran, gelatin, polyethylene glycol, and polyoxyethylene or polyoxypropylene ethers;
(q) Surfactants selected from ionic or non-ionic surface-active agents including anionic surfactants such as sodium lauryl sulfate, sodium dodecylbenzene sulfonate, dialkyl sodium sulfosuccinates, and alkyl sulfates; cationic surfactants such as benzalkonium chloride, benzethonium chloride, cetrimonium bromide, and stearyl dimethylbenzyl ammonium chloride; and non-ionic surfactants such as polysorbates, lecithin, and polyoxyethylene ethers;
(r) Anti-foaming agents selected from the group comprising simethicone, polydimethylsiloxane, organomodified silicones, mineral oil, vegetable oil, polypropylene glycol, fatty alcohols, aliphatic esters, triglycerides, and combinations thereof;
(s) Dispersing agents selected from the group comprising polysorbates, lecithin, polyvinylpyrrolidone, poloxamers, sodium lauryl sulfate, sodium citrate, sodium carboxymethylcellulose, phospholipids, or amphiphilic polymers;
(t) Wetting agents selected from the group comprising polysorbates, sodium lauryl sulfate, poloxamers, lecithin, polyoxyethylene sorbitan esters, polyvinylpyrrolidone, sorbitan esters, cetyl alcohol, stearyl alcohol, glycerol monostearate, polyethylene glycols, and sodium dioctyl sulfosuccinate; and combinations thereof;
(u) Emulsifying agents selected from the group comprising polysorbates (PS such as PS 20, PS 40, PS 60, PS 80), lecithin, sorbitan esters (including sorbitan monolaurate, sorbitan monooleate, sorbitan monostearate), polyoxyethylene sorbitan esters, poloxamers, sodium lauryl sulfate, cetyl alcohol, stearyl alcohol, glyceryl monostearate, polyethylene glycol esters, mono- and diglycerides, egg yolk phospholipids, sodium stearoyl lactylate, sodium caseinate, and combinations thereof; and
(v) Film forming agents selected from the group comprising cellulose-based derivatives such as cellulose acetate phthalate (CAP), hydroxypropyl methylcellulose phthalate (HPMCP), hydroxypropyl methylcellulose acetate succinate (HPMCAS), cellulose acetate trimellitate (CAT), and carboxymethylcellulose derivatives; vinyl polymers such as polyvinyl acetate phthalate (PVAP); acrylic copolymers including methacrylic acid copolymers commercially available under the Eudragit® trade name (e.g., Eudragit® L 100-55, Eudragit® L, Eudragit® S, Eudragit® FS), and poly(methacrylic acid-co-methyl methacrylate); naturally derived polymers such as purified resin shellac, zein, polysaccharides including sodium alginate, pectin, guar gum, chitosan, and gellan gum; specialized capsule polymers such as DRCaps™; and mixtures thereof.

### 1. Vehicles

According to some aspects, the liquid pharmaceutical composition of the present invention comprises a vehicle. The terms “vehicle”, “pharmaceutically acceptable vehicle”, “diluent” or “filler” as used herein is any ingredient or mixture or combination of ingredients, either in liquid form or solid form, that can be dissolved, homogenized or suspended, to increase the volume of the liquid composition of present invention. Non-limiting examples of vehicles are water (preferably purified water for oral or intranasal dosage form and water for injection for parenteral dosage form), sorbitol, sucrose, sugar, ethanol, mannitol, xylitol, propylene glycol, polyethylene glycol, glycerin, lactose, cellulose, starch, triglycerides, or oil. In some embodiments of the present invention, the concentration of vehicles ranges from about 5% to about 99.99%, from about 10% to about 99.99%, from about 20% to about 99.99%, from about 30% to about 99.99%, from about 40% to about 99.99%, from about 50% to about 99.99%, from about 50% to about 99.99%, from about 60% to about 99.99%, from about 70% to about 99.99%, from about 80% to about 99.99%, from about 90% to about 99.99% based on the total weight of the composition. In some embodiments, the preferred vehicles are selected from the group comprising of water, propylene glycol and polyethylene glycol.

In an embodiment of the present invention, vehicles are added in sufficient quantity (quantum satis or q.s.) to achieve the target concentration of the liquid formulation of edaravone, after accounting for the amount of all other ingredients.

In some embodiments of the present invention, ethanol is not a desirable ingredient due to handling risk during manufacturing, safety in pediatrics, risk of drug abuse, drug interactions, and potential risk of crystallization due to ethanol evaporation during in-use of multidose containers. The formulation of the present invention comprises ethanol at concentration of less than about 40% w/v, preferably less than about 35% w/v, preferably less than about 30% w/v, preferably less than about 25% w/v, preferably less than about 20% w/v, preferably less than about 15% w/v, preferably less than about 12.5% w/v, preferably less than about 10% w/v, preferably less than about 5% w/v, preferably less than about 2% w/v, preferably about less than about 1% w/v, preferably less than about 0.5% w/v. In most preferred embodiments, the formulation of the present invention is essentially free from ethanol, comprising ethanol at concentration of less than about 10% w/v, more preferably less than about 5% w/v, more preferably less than about 2% w/v.

In some embodiments of the present invention, the vehicle comprises organic solvent selected from propylene glycol, polyethylene glycol, glycerin, or combination thereof, wherein the concentration of the said organic solvent is at least about 40% w/v, optionally at least about 50% w/v, optionally at least about 60% w/v, optionally in the range from about 40% w/v to about 90% w/v. In certain embodiments, the weight ratio of the said organic solvent system to ethanol (if present), is greater than about 1:1, preferably greater than about 2:1, preferably greater than about 2.5:1, preferably greater than about 3:1, preferably greater than about 4:1, preferably greater than about 5:1, and preferably greater than about 10:1 (organic solvent:ethanol % w/w).

In some embodiments, the edaravone formulation of the present invention comprises polyhydric alcohol (such as PG, PEG, Glycerol) with low concentration of oxidative impurities. Such impurities are peroxides (e.g., hydrogen peroxide, organic hydroperoxides), aldehydes (e.g., formaldehyde, acetaldehyde), and other reactive species often found in polyhydric alcohols. In one embodiment, the vehicle may have a peroxide content of less than about 1000 ppm, more preferably less than about 500 ppm, more preferably less than about 100 ppm, more preferably less than about 50 ppm, more preferably less than about 25 ppm, more preferably less than about 10 ppm, and most preferably less than about 5 ppm.

In some embodiments, the edaravone formulation of the present invention comprises polyhydric alcohol (such as PG, PEG, Glycerol) with low concentration of aldehyde impurities (e.g. formaldehyde, acetaldehyde), most commonly formaldehyde. In one embodiment, the vehicle may have a formaldehyde content of less than about 1000 ppm, more preferably less than about 500 ppm, more preferably less than about 100 ppm, more preferably less than about 50 ppm, more preferably less than about 30 ppm, more preferably less than about 10 ppm, and most preferably less than about 5 ppm.

In some embodiments of the present invention, oil is used as vehicle, specifically to dissolve edaravone to prepare oil-based solutions and emulsion or microemulsion.

In some embodiments of the present invention, oil is used as vehicle, specifically to dissolve edaravone to prepare oil-based solutions, emulsions, or as base for liquid filled capsules. Oil used in a liquid formulation of present invention is selected from the group comprising of, without limitation, olive oil, almond oil, fish oil, avocado oil, sesame oil, canola oil, sunflower oil, castor oil, soybean oil, coconut oil, anise oil, apricot kernel oil, cinnamon oil, corn oil, cottonseed oil, rapeseed oil, mustard oil, palm oil, peanut oil, macadamia oil, beechnut oil, brazil nut oil, cashew oil, hazel nut oil, pecan oil, pine nut oil, pistachio oil, walnut oil, pumpkin seed oil, rice-bran oil, grapefruit seed oil, lemon oil, orange oil, bitter gourd oil, bottle gourd oil, buffalo gourd oil, butternut squash seed oil, edusi seed oil, watermelon seed oil, acai seed oil, black seed oil, blackcurrent seed oil, borage seed oil, evening primrose oil, flaxseed oil, amaranth oil, apricot oil, apple seed oil, argan oil, babassu oil, ben oil, borneo tallow nut oil, cape chestnut oil, Citrus Sinensis flower oil, clove oil, coriander oil, eucalyptus oil, lanolin oil, mineral oil, nutmeg oil, peppermint oil, spearmint oil, vegetable oil, animal oil, hydrogenated vegetable oil, polyoxyl vegetable oil, apricot kernel oil PEG-6 esters, Citrus sinensis flower oil, palm kernel oil glycerides (hydrogenated), corn oil mono- and di-glycerides (c18), hydrogenated castor oil, hydrogenated coconut oil, hydrogenated cottonseed oil, hydrogenated palm oil, hydrogenated soybean oil, lanolin alcohol-mineral oil, lavender oil, light mineral oil, lime oil, natural oil, orange oil terpeneless, PEG-40 castor oil, PEG-60 hydrogenated castor oil, pine needle oil (Pinus sylvestris), polyoxyl 35 castor oil, polyoxyl 40 hydrogenated castor oil, sassafras oil, liquid triglyceride of 6 to 12 carbon chain length, thyme oil, turpentine oil, hydrogenated vegetable oil glyceride, Miglyol® 818, Miglyol® 812, Miglyol® 840, Labrafac®, Captax® 385, Capmul®, Imwitor® 988, Imvitor® 948, oleic acid, linoleic acid, Maisine® 35-1, Capryol® 90, Maisine® CC, Peceol™ or combination thereof. The concentration of oil in the disclosed liquid formulation of edaravone ranges from about 1% to about 99.99%, optionally from about 30% to about 99.99% based on the total weight of the composition depending on concentration of edaravone in the formulation and formulation type. For example, microemulsion of edaravone with concentration of about 1 mg/mL may need lower concentration of oil than oil based oral solution.

### 2. Solvents or Solubilizers

The liquid pharmaceutical composition of the present invention may comprise one or more solubilizers. The term “solubilizer” or “solvent” as described herein refers broadly to any ingredient, vehicle, or excipient that enhances, maintains, or aids the solubility of edaravone in the liquid medium, or otherwise prevents precipitation of the active ingredient. While specific classes of excipients are defined in detail elsewhere in this disclosure, such as vehicles, surfactants, complexing agents, and crystallization inhibitors, it is understood that such ingredients may simultaneously function as solubilizers in the context of the present invention. Accordingly, suitable solubilizers include, without limitation, water-miscible organic solvents such as propylene glycol, glycerin, polyethylene glycol (PEG, such as PEG 200, PEG 300, PEG 400, PEG 600, PEG 1000, PEG 3350, PEG 4000, etc.), ethanol, and diethylene glycol monoethyl ether (Transcutol®). Additionally, functional excipients with solubilizing properties include ingredients described herein as surfactants (e.g., polysorbates, polyoxyl castor oils), complexing agents (e.g., cyclodextrins), crystallization inhibitors (e.g., PVP, HPMC), and hydrotropes. Other suitable solubilizers include dimethyl sulfoxide (DMSO), N-methyl-2-pyrrolidone (NMP), glycofurol, and Vitamin E TPGS.

In some embodiments of the present invention, the concentration of solubilizers ranges from about 0.01% to about 99.99% based on the total weight of the composition. In embodiments where the solubilizer acts as a primary vehicle or co-solvent (e.g., propylene glycol or polyethylene glycol), the concentration may range from about 5% to about 99.99%, from about 10% to about 99.99%, from about 20% to about 99.99%, from about 30% to about 99.99%, from about 40% to about 99.99%, from about 50% to about 99.99%, or from about 60% to about 90%. In other embodiments where the solubilizer acts as a functional excipient (e.g., a surfactant, polymer, or complexing agent), the concentration may range from about 0.01% to about 70%, preferably from about 0.1% to about 50%, preferably from about 0.1% to about 20%, preferably from about 0.1% to about 10%, or preferably from about 0.1% to about 5% based on the total weight of the composition.

### 3. Preservatives

According to some aspects, the liquid pharmaceutical composition of the present invention comprises one or more preservative(s). The term “preservative” used herein refers to any ingredient or mixture or combination of ingredients added to prevent or inhibit the growth of microorganisms, thereby ensuring the safety and shelf-life of the product. Preservatives are especially important in multi-dose preparations, where repeated access can introduce contaminants, and their inclusion helps maintain the sterility and integrity of the formulation throughout its intended use. In an embodiment, the pharmaceutical composition of the liquid formulation of edaravone comprises one or more preservative(s) selected from the group comprising of benzoic acid and the sodium or potassium salts thereof, ethanol, isopropanol, methanol, butyl alcohol, benzalkonium chloride, benzyl alcohol, benzethonium chloride, butylparaben, cetylpyridinium chloride, chlorobutanol, chlorocresol, cresol, dehydroacetic acid, ethylparaben, ethylparaben sodium, methylparaben, methylparaben sodium, phenol, phenylethyl alcohol, phenylmercuric acetate, phenylmercuric nitrate, potassium benzoate, potassium sorbate, propylparaben, propylparaben sodium, sodium dehydroacetate, sodium propionate, sorbic acid, thimerosal, thymol, or combinations thereof.

In some embodiments of the present invention, the concentration of preservatives ranges from 0.001% to about 10%, preferably from about 0.01% to about 5%, preferably from about 0.01% about 2%, preferably from about 0.01% to about 1%, preferably from about 0.01% to about 0.5% based on the total weight of the composition.

In some other embodiments of the present invention, the concentration of preservatives ranges from 0.1% to about 10%, preferably from about 0.1% to about 5%, preferably from about 0.1% about 2%, preferably from about 0.1% to about 1%, preferably from about 0.1% to about 0.5% based on the total weight of the composition.

In some embodiments, liquid formulation of the present invention may contain an ingredient or a group of ingredients at concentration that is sufficient to prevent microbial growth by generating higher osmotic pressure. Non-limiting examples for such ingredients are sugars (sorbitol, mannitol, xylitol, dextrose, sucrose, lactose, etc.) at concentration from about 5% to about 90%, preferably from about 10% to about 70%, preferably from about 40%; and salts (such as sodium chloride, potassium chloride, etc.) at concentration from about 0.5% to about 10%, preferably from about 1% to about 5%. In some other embodiments, edaravone may be present at a concentration that prevents microbial growth and act as self-preservative and it is still within the scope of the present invention. In yet other embodiments of the present invention, non-aqueous solvent used as vehicle, such as polyethylene glycol, propylene glycol, glycerin, ethanol or combination thereof at quantity enough (for example from about 5 to about 99% w/v, preferably from about 50 to about 99% w/v, preferably more than about 50% of the total weight of the composition) preventing microbial growth acting as preservative and it is still within the scope of the invention.

### 4. Sweeteners

According to some aspects, the liquid pharmaceutical composition of the present invention comprises one or more sweetening agent(s). The terms “sweetening agent” or “sweetener” refers to both bulk (caloric) and intense (non-caloric) sweeteners, which impart a sweet taste to the preparation. Non-limiting examples of sweeteners include glucose, sucralose, neotame, aspartame, advantame, trehalose, fructose, xylose, dextrose, galactose, tagatose, maltose, sucrose, glycerol, dulcitol, mannitol, lactitol, sorbitol, xylitol, saccharine or the corresponding sodium, potassium or calcium salt, cyclamate or the corresponding sodium or calcium salt, acesulfame or the potassium salt thereof, dulcin or ammonium glycyrrhizinate (Magnasweet® 110 or similar), alitame, inulin, steviol glycoside, isomalt, neohesperidin dihydrochalcone, thaumatin and the like or any combinations thereof.

In some embodiments of the present invention, the concentration of sweeteners ranges from about 0.001% to about 90%, preferably from about 0.001% to about 40%, preferably from about 0.01% to about 30%, preferably from about 0.01% to about 20%, preferably from about 0.01% to about 10%, preferably from about 0.01% to about 5%, preferably from about 0.01% to about 2%, preferably from about 0.1% to about 2% based on the total weight of the composition.

In some other embodiments of the present invention, the concentration of sweeteners ranges from about 0.01% to about 70%, preferably from about 0.1% to about 40%, preferably from about 1% to about 40%, preferably from about 5% to about 40%, preferably from about 10% to about 40%, preferably from about 20% to about 40%, preferably from about 30% to about 40%, preferably from about 10% to about 20%, preferably from about 20% to about 30%, preferably from about 5% to about 15% based on the total weight of the composition. In some embodiments of the present invention, the intense sweetener may require less quantity for taste masking (for example sweetener like aspartame, neotame, advantame, sucralose, saccharin, etc.) while low-intensity sweetener (such as glucose, sorbitol, xylitol, mannitol, etc.) may require larger quantity of sweetener for taste masking.

### 5. Flavors

According to some aspects, the liquid pharmaceutical composition of the present invention comprises one or more flavoring agent(s). The terms “flavoring agent,” or “flavor” as used herein, refer to an agent or a mixture of agents that adds flavor to a mixture. A flavoring agent is selected from the group comprising of a natural flavor, an artificial flavor, and mixtures thereof. Non-limiting examples of flavoring agents are vanilla, citrus oil, including lemon, orange, grape, lime and grapefruit, and fruit essences, including apple, banana, pear, peach, strawberry, raspberry, cherry, plums pineapple, apricot, peppermint, tutti frutti flavor and so forth and the like or any combinations thereof. Solid forms, such as spray dried forms of flavoring agents, may also be useful in the liquid dosage forms disclosed herein.

In some embodiments of the present invention, the concentration of flavoring agent ranges from about 0.001% to about 5%, preferably from about 0.001% to about 2%, preferably from about 0.001% to about 1%, preferably from about 0.001% to about 0.1%, preferably from about 0.01% to about 0.1% based on total weight of the composition.

In some other embodiments of the present invention, the concentration of flavoring agent ranges from about 0.01% to about 5%, preferably from about 0.01% to about 5%, preferably from about 0.1% to about 5%, preferably from about 1% to about 5%, preferably from about 2% to about 5% based on total weight of the composition.

### 6. pH Adjusting Agents

According to some aspects, the liquid pharmaceutical composition comprises one or more pH adjusting agent(s). As used herein, the term “pH adjusting agent” refers to a component or combination of components sufficient to adjust a pharmaceutical composition's pH. According to some aspects, the pH adjusting agent is sufficient to raise or lower the pharmaceutical composition's initial pH to a pharmaceutically acceptable range (i.e., not toxic or producing unacceptable side effects). Additionally, or alternatively, the pH adjusting agent may be sufficient to maintain the pharmaceutical composition's pH in a pharmaceutically acceptable range over a certain period of shelf life. Example of pH adjusting agents includes without limitation, acetic acid, adipic acid, ammonia, ascorbic acid, barium hydroxide, benzoic acid, calcium hydroxide, carbonic acid, cesium hydroxide, citric acid, formic acid, fumaric acid, gluconic acid, glutamic acid, hydrochloric acid, isothionic acid, lactic acid, lithium hydroxide, malic acid, maleic acid, mandelic acid, methane sulfonic acid, monopotassium phosphate, monosodium phosphate, mucic acid, nitric acid, oxalic acid, p-toluenesulfonic acid, pantothenic acid, phosphoric acid, phthalic acid, potassium hydroxide, pyrophosphoric acid, rubidium hydroxide, sodium hydroxide, strontium hydroxide, succinic acid, sulfuric acid, tartaric acid, or combination thereof. In one example, the pharmaceutical composition comprises hydrochloric acid, sodium hydroxide, or a combination thereof.

In some embodiments of the present invention, the concentration of pH adjusting agent ranges up to about 10%, preferably up to about 8%, preferably up to about 5%, preferably up to about 2% based on total weight of the composition. In some embodiments of the present invention, pH adjusting agent (such as citric acid) may be used in mole ratio of edaravone such as from about 0.5 to about 1.5:1.0 (pH adjusting agent:edaravone), optionally from about 0.9 to 1.1:1.0, optionally from about 1.0 to 1.1:1.0, optionally from about 1.00 to 1.05:1.00, optionally ≥1.00:1.00.

In some other embodiments of present invention, pH adjusting agent donating more than one proton or accepting more than one anion (such as phosphoric acid acid) may have reduced ratio by dividing provided mole above by number of donating anions.

In some other embodiments of the present invention, the concentration of pH adjusting agent may depends on the pH of formulation and quantity to be used is generally considered as “quantity sufficient (q.s.) to target pH”.

According to some aspects, each of the pH adjusting agents may together or independently have a concentration in the range from about 0.0001 N to about 20 N, preferably from about 0.01 N to about 1 N.

According to some aspects, the pharmaceutical composition may be free of pH adjusting agents as described herein. It should be understood that the pH-adjusting agent-free pharmaceutical composition may have an initial pH within a pharmaceutically acceptable pH range, as described herein and/or may have a pH in a pharmaceutically acceptable range over a certain period of shelf life, as described herein.

### 7. Buffering Agents

According to some aspects, the liquid pharmaceutical composition of the present invention comprises one or more buffering agent(s). The term “buffering agent”, also known as buffer system, refers to ingredients that helps maintaining the pH of formulation. Examples of buffering agents include, without limitation, salts of citric acid, tartaric acid, ascorbic acid, phosphoric acid, acetic acid, carbonic acid, phthalic acid, succinic acid, glutamic acid, benzoic acid, gluconic acid, formic acid, fumaric acid, lactic acid, isothionic acid, malic acid, maleic acid, mandelic acid, nitric acid, mucic acid, oxalic acid, pantothenic acid, p-toluenesulfonic acid, methane sulfonic acid, sulfuric acid, hydrochloric acid, or combination thereof. Additionally, pharmaceutically acceptable amino acids such as methionine, cysteine, glycine, etc. can also be used as buffering agents.

In some other embodiments of the present invention, the concentration of buffering agent ranges from 0.001% to 10%, preferably from about 0.001% to about 5%, preferably from about 0.001% to about 2%, preferably about 0.001% to about 1%, preferably about 0.001% to 0.5%, preferably about 0.001% to about 0.25% based on the total weight of the composition.

In some other embodiments of the present invention, the concentration of buffering agent ranges from 0.01% to 10%, preferably from about 0.01% to about 5%, preferably from about 0.01% to about 2%, preferably about 0.01% to about 1%, preferably about 0.01% to 0.5%, preferably about 0.01% to about 0.25% based on the total weight of the composition.

In some other embodiments of the present invention, the concentration of buffering agent ranges from 0.1% to 10%, preferably from about 0.1% to about 5%, preferably from about 0.1% to about 2%, preferably about 0.1% to about 1%, preferably about 0.1% to 0.5%, preferably about 0.1% to about 0.25% based on the total weight of the composition.

It is understood that a higher quantity of acid or base can be used in higher than range mentioned in the disclosure considering that they neutralize the effect of each other and are still within the scope of the invention. In some embodiments of the present invention, the concentration of pH adjusting agent, or buffering agent is net quantity after neutralization by counter ions.

### 8. Viscosity Adjusting Agents

According to some aspects, the liquid pharmaceutical composition of the present invention comprises one or more viscosity adjusting agent. As used herein, the term “viscosity adjusting agent” refers to any compound or combination of compounds added to a formulation of the present invention to modify or maintain its viscosity, ensuring optimal flow, pourability, stability, and ease of administration of the final liquid formulation.

A viscosity-adjusting agent can be utilized to achieve the desired formulation consistency based on the specific requirements of the end user. For instance, some individuals with ALS presents dysphagia. In such cases, oral liquids with lower viscosity increase the risk of aspiration into the lungs, making higher viscosity liquids more appropriate. In another example, a modest increase in the viscosity of nasal spray formulations can extend their residence time on the nasal mucosa, potentially facilitating more thorough absorption.

In one embodiment, viscus non-aqueous solvent such as propylene glycol, glycerin, polyethylene glycol, tocopherol or vitamin E (such as α-tocopherol) may be used to increases or adjust the viscosity for desired properties. In another embodiment, alcohol (such as ethanol) may be used to reduce the viscosity of the formulation. In yet another embodiment, combination of high and low viscosity solvent may be combined with making aqueous or non-aqueous liquid formulation of the present invention with desired viscosity and/or other characteristics.

In certain embodiments, achieving bioequivalence between an oral liquid and a solid dosage form, such as a tablet, or oral suspension requires modulating the absorption rate of edaravone. As a solution edaravone may be prone to rapid absorption from an oral route of administration, which can result in a Cₘₐₓ that significantly exceeds that of the reference tablet or reference suspension. To mitigate this pharmacokinetic mismatch, a viscosity-adjusting agent may be incorporated into the liquid formulation of the present invention. This agent increases the viscosity of the vehicle, thereby slowing gastric emptying and reducing the drug's diffusion rate to the gastrointestinal mucosa. This controlled slowing of absorption ensures the Cₘₐₓ of the liquid formulation falls within the bioequivalent range of the tablet or suspension.

According to some aspects, the liquid pharmaceutical composition of the present invention comprises a viscosity-adjusting agent from the class of cellulose derivatives. The term “cellulose derivative” refers to semi-synthetic polymers derived from natural cellulose that hydrate and swell in water to form a viscous, three-dimensional polymer network, thereby controlling the formulation's flow properties and the drug's rate of absorption. Examples include, without limitation, Hydroxypropyl Methylcellulose (HPMC), Sodium Carboxymethylcellulose (Sodium CMC), Hydroxyethyl Cellulose (HEC), Methylcellulose, or combinations thereof. In certain embodiments, the concentration of such cellulose derivatives may range from about 0.001% w/v to about 10.0% w/v, depending on the nature of specific agent and desired viscosity.

According to some aspects, the liquid pharmaceutical composition of the present invention comprises a viscosity-adjusting agent from the class of natural gums. The term “natural gum” refers to high molecular weight polysaccharides derived from natural sources, such as microbial fermentation or botanical origins, which are efficient at increasing viscosity at low concentrations and may impart specific rheological properties such as pseudoplasticity. Examples include, without limitation, Xanthan Gum, Guar Gum, Acacia, Tragacanth, Carrageenan, or combinations thereof. In certain embodiments, the concentration of such natural gums may range from about 0.001% to about 2.0% w/v, optionally from about 0.001% to about 0.5% w/v, with agents like xanthan gum often being effective at the lower end of this range.

According to some aspects, the liquid pharmaceutical composition of the present invention comprises a viscosity-adjusting agent from the class of synthetic polymers. The term “synthetic polymer” refers to man-made polymers engineered with specific properties to modify the rheology of the formulation, often providing high viscosity with unique characteristics such as pH-dependent or temperature-dependent thickening. Examples include, without limitation, Carbomers, Polyvinylpyrrolidone (PVP), Poloxamers, Polyvinyl Alcohol (PVA), or combinations thereof. In certain embodiments, the concentration of such synthetic polymers varies by type; for example, high-efficiency agents like carbomers may be used from about 0.05% to about 1.0% w/v, whereas agents like polyvinylpyrrolidone may be used in a range from about 1.0% to about 10.0% w/v.

In some embodiments of the present invention, the concentration of viscosity adjusting agent ranges from about 0.001% to about 10%, from about 0.01% to about 5%, from about 0.1% to about 2%, from about 0.1% to about 1% by total weight of the composition, depending on the desired consistency and properties of final formulation. The quantity of viscosity adjusting agents used in this invention may vary depending on the specific properties and type of agent selected. For instance, a viscous liquid edaravone formulation of present invention utilizing 1% w/v hypromellose K4M can also be produced with hypromellose K15M or K100M to achieve similar viscosity; however, the required quantity of hypromellose K15M or K100M will be substantially lower than that used in the K4M-based formulation.

### 9. Antioxidants

In some embodiments of the present invention, the liquid pharmaceutical composition of the present invention comprises one or more antioxidants. The term “antioxidant” refers to agents that protect any ingredients of the composition from oxidative degradation. Examples include, without limitation, methionine, sodium bisulfite, potassium bisulfite, ammonium bisulfite, sodium metabisulfite, potassium metabisulfite, sodium ascorbate, ascorbic acid, butylated hydroxytoluene, butylated hydroxyanisole, cysteine, glutathione, monothioglycerol, propyl gallate, gallic acid, sodium sulfite, potassium sulfite, calcium sulfite, tocopherol (such as alpha-tocopherol) or its salt and esters (such as D-alpha-tocopheryl polyethylene glycol 1000 succinate), or combination thereof. It is understood that some of the antioxidants, as disclosed in the examples of the present disclosure, may surprisingly degrade edaravone in specific environment and/or at certain concentration and should be used carefully. It is understood that sodium metabisulfite added in the experiments and examples may be present as sodium bisulfite after interacting with water, and similarly for potassium metabisulfite.

In some embodiments of the present invention, the concentration of antioxidants ranges from about 0.001% to about 5%, optionally from about 0.01% to about 2%, optionally from about 0.01% to about 1%, optionally from about 0.01% to about 0.5%, optionally from about 0.01% to about 0.1% based on total weight of the composition.

In some embodiments of the present invention, the concentration of antioxidants ranges from about 0.05% to about 2%, optionally from about 0.1% to about 2%, optionally from about 0.1% to about 2%, optionally from about 0.1% to about 1%, optionally from about 0.1% to about 0.5% based on total weight of the composition.

### 10. Tonicity Adjusting Agents

In some embodiments of the present invention, the liquid pharmaceutical composition of the present invention comprises one or more tonicity adjusting agent. As used herein, the term “tonicity adjusting agent” refers to any ingredient or combination of ingredients added to the pharmaceutical formulation of the present invention to adjust or contributing to its osmotic pressure to the target, thereby ensuring that the formulation is hypotonic or isotonic or near-isotonic or hypertonic based on intended application and/or site of administration with respect to physiological fluids, such as nasal mucosa, when administered intranasally, or to a soft tissue or muscle when administered by intramuscular route, or into the blood when administered intravenously. Tonicity adjusting agents contribute to patient comfort, prevent irritation, may improve absorption, and help maintain the stability and efficacy of the formulation upon administration. Non-limiting examples of tonicity adjusting agents that may be used in the pharmaceutical compositions of the present invention include alanine, arginine, betaine, boric acid, calcium chloride (CaCl₂), calcium lactate, dextran, dextrose, fructose, glycerin, glycine, glucose, histidine, hydroxyethyl starch, lactose, lysine, magnesium chloride, magnesium sulfate (MgSO₄), maltose, mannitol, polyethylene glycol (PEG), potassium chloride (KCl), potassium citrate, proline, propylene glycol, raffinose, ribose, sodium acetate, sodium benzoate, sodium bicarbonate (NaHCO₃), sodium citrate, sodium chloride (NaCl), sodium gluconate, sodium lactate, sodium phosphate (NaH₂PO₄ or Na₂HPO₄), sodium sulfate (Na₂SO₄), sodium tartrate, sodium thiosulfate (Na₂S₂O₃), sorbitol, sucrose, taurine, trehalose, tromethamine (Tris), xylitol, etc., as well as mixtures thereof. In some embodiments of the present invention, the tonicity adjusting agent is ionic (such as salts) or non-ionic (such as sugars). In some embodiments, the concentration of tonicity adjusting agents ranges from about 0.1% to about 10%, from about 0.2% to about 8%, from about 0.5% to about 5% by weight based on the total composition, with the precise amount selected to achieve the desired osmolality, for example, from about 100 mOsmol/Kg to about 1000 mOsmol/Kg, optionally from about 250 mOsmol/Kg to about 750 mOsmol/Kg, optionally from about 250 mOsmol/kg to about 350 mOsmol/kg. The specific selection, combination, and concentration of tonicity adjusting agents is tailored according to the overall composition and intended route of administration to ensure optimal therapeutic performance and patient tolerability. In some embodiments of the present invention, edaravone in dissolved form also contributes to the osmolality of the final formulation.

At given concentration of edaravone in vehicle, it will also act as tonicity adjusting agent to achieve target osmolality and it is within the scope of present invention. In one embodiment, the present invention provides edaravone liquid formulation suitable for intranasal administration comprising of edaravone at concentration of about 50 mg/mL, that may have osmolality of about 300 mOsmol/Kg, which is isotonic to bodily fluid and may not need to add additional tonicity adjusting agent. In this example, edaravone acts as tonicity adjusting agent. In some other embodiments, where higher target tonicity is desired for improve absorption through mucosal membrane (e.g. 750 mOsmol/Kg), solvent (e.g. propylene glycol) may act as tonicity adjusting agent in addition to vehicle.

In some embodiments, the present invention provides a edaravone liquid formulation intended for intranasal and/or parenteral administration, comprising edaravone at a concentration that achieves patient-tolerable tonicity without causing significant discomfort and without need of additional tonicity adjusting agent.

### 11. Chelating Agents

In some embodiments, the liquid pharmaceutical composition of the present invention comprises one or more chelating agents. The term “chelating agent” refers to compounds that helps to stabilize the formulation by means of chelating undesired compounds or elements or ions. Suitable chelating agents which may be used in the present invention include, but are not limited to, edetate disodium (EDTA); edetate trisodium, edetate tetrasodium, edetate calcium disodium; and diethylene amine pentaacetate or derivatives thereof. In certain preferable embodiments, the formulations comprise disodium edetate. In some embodiments, any hydrate or anhydrous or polymorphs of the said chelating agents are within the scope of the invention.

In some other embodiments, hydrocarboxylic acids such as citric acid, amino acids such as histidine, siderophore such as deferoxamine, may also be used as chelating agents in the liquid formulation of the present invention.

In some embodiments of the present invention, the concentration of chelating agents ranges from 0.001% to 5%, preferably from about 0.001% to about 2%, preferably from about 0.001% to about 1%, preferably from about 0.001% to about 0.5%, preferably from about 0.001% to about 0.1% based on the total weight of the composition.

In some other embodiments of the present invention, the concentration of chelating agents ranges from 0.01% to 5%, preferably from about 0.01% to about 2%, preferably from about 0.01% to about 1%, preferably from about 0.01% to about 0.5%, preferably from about 0.1% to about 0.2% based on the total weight of the composition.

### 12. Permeability Enhancing Agents

According to some aspects, the liquid pharmaceutical composition of the present invention comprises one or more permeability enhancers. As used herein, the terms “permeation enhancer” or “permeability enhancer” refers to an ingredient or group of ingredients that are incorporated into the liquid formulation of the present invention to increase the permeability of the edaravone across biological membranes. Permeation enhancers function by temporarily modifying the barrier properties of the membrane, facilitating improved absorption or bioavailability of the drug. Non-limiting examples of permeation enhancers include, without limitation: alcohol, arachidonic acid, benzethonium chloride, benzethonium bromide, benzalkonium chloride, capric acid, caproic acid, carvone, cetylpyridium chloride, chitosans, citric acid, 6-cyclohexyl-1-hexyl-P-D-maltopyranoside, n-decyl-P-D-maltopyranoside, dimethyl sulfoxide, dodecyl dimethyl aminopropionate, 1-O-n-Dodecyl-P-D-maltopyranoside, dodecylpolyethyleneglycolether, edetate disodium dihydrate, enanthic acid, glyceryl monooleate, glyceryl monostearate, glycofurol, isopropyl myristate, isopropyl palmitate, pelargonic acid, lanolin, lauric acid, light mineral oil, limonene, linoleic acid, lysine, menthol, myristic acid, myristyl alcohol, oleic acid, oleyl alcohol, palmitic acid, peppermint oil, polyoxyethylene alkyl ethers, polyoxylglycerides, polysorbates, pyrrolidone, sodium caprate, sodium desoxycholate, sodium deoxyglycolate, sodium glycocholate, sodium hydroxybenzoyal amino caprylate, sodium lauryl sulfate, sodium taurocholate, stearic acid, thymol, tricaprylin, triolein, undecylenic acid, and combinations thereof. In some embodiments of the present invention, tonicity adjusting agent added to achieve hypertonic liquid (more than about 300 mOsmol/Kg, e.g. 750 mOsmol/Kg) may act as permeation enhancer.

Without wishing to be bound by any particular theory, in certain embodiments, the permeability enhancer functions as an inhibitor of efflux transporters, specifically P-glycoprotein (P-gp). Edaravone is a known substrate of P-gp, an efflux pump expressed in the intestinal epithelium and the blood-brain barrier that actively transports substrates back into the intestinal lumen or blood, thereby limiting systemic bioavailability and central nervous system (CNS) penetration. By incorporating excipients that inhibit P-gp, the present invention reduces this active efflux, thereby increasing the net absorption and bioavailability of edaravone. The mechanism of P-gp inhibition by these excipients may include competitive inhibition of the transporter, inhibition of the P-gp ATPase activity, alteration of membrane fluidity which impairs transporter function, or downregulation of P-gp expression.

In some other embodiments, the formulation comprises excipients selected for their dual ability to solubilize edaravone and inhibit P-gp efflux, including but not limited to: polyoxyl castor oils (e.g., Cremophor® EL, Cremophor® RH40, Kolliphor® EL), polysorbates (e.g., Polysorbate 20, Polysorbate 80), vitamin E derivatives (e.g., D-alpha-tocopheryl polyethylene glycol 1000 succinate or Vitamin E TPGS), poloxamers (e.g., Poloxamer 188, Poloxamer 407), polyethylene glycols (e.g., PEG 300, PEG 400), macrogol 15 hydroxystearate (e.g., Solutol® HS 15), caprylocaproyl polyoxyl-8 glycerides (e.g., Labrasol®), gelucire (e.g., Gelucire® 44/14, Gelucire® 50/13), Transcutol® (diethylene glycol monoethyl ether), bioflavonoids (e.g., quercetin, curcumin, naringin), and alkaloids (e.g., piperine).

In some embodiments of the present invention, the concentration of permeation enhancer ranges from about 0.001% to about 10%, preferably from about 0.001% to about 20%, preferably from about 0.001% to about 5%, or preferably from about 0.001% to about 2% based on the total weight of the composition. The precise concentration may be adjusted depending on desired permeability enhancement, physical properties of the formulation, and compatibility with other ingredients.

### 13. Mucoadhesive Agents

According to some aspects, the liquid pharmaceutical composition of the present invention comprises one or more mucoadhesive agents. As used herein, the term “mucoadhesive agent” refers to an ingredient or group of ingredients that enhance the adhesion of the formulation to mucosal surfaces, thereby prolonging the residence time and improving the efficacy of the active pharmaceutical ingredient of the present invention. Mucoadhesive agents function by forming physical or chemical interactions with the mucin layer covering mucosal tissues. Examples of mucoadhesive agents include, without limitation, natural polymers such as chitosan, alginate, hyaluronic acid, gelatin, tragacanth, xanthan gum, guar gum, and pectin; synthetic and semi-synthetic polymers such as carbomers (Carbopol) of all grades (such as Carbopol 934, Carbopol 940, Carbopol 971P, and others), polyvinyl alcohol (including all pharmaceutical grades), polyvinylpyrrolidone (such as K12, K30, and K90 grades), polyethylene oxide (including Sentry Polyox grades such as WSR N10, N80, N750, and N205), polyacrylic acid (all USP and NF grades), hydroxypropyl methylcellulose (HPMC grades such as E3, E5, K4M, K15M, and K100M), methylcellulose (USP and food grades), carboxymethyl cellulose (including sodium carboxymethyl cellulose grades like low, medium, and high viscosity types), and poloxamers (including grades such as Poloxamer 188 and 407); as well as cellulose derivatives (all grades), sodium alginate, and acacia gum. In some embodiments of the present invention, the concentration of mucoadhesive agent ranges from about 0.001% to about 10%, preferably from about 0.01% to about 5%, preferably from about 0.1% to about 2%, preferably from about 0.1% to about 1%, and more preferably from about 0.1% to about 0.5% based on the total weight of the composition. The precise concentration may be adjusted depending on desired mucoadhesive strength, physical properties of the formulation, and compatibility with other ingredients.

### 14. Stabilizers

According to some aspects, the liquid pharmaceutical composition of the present invention comprises one or more stability enhancing agent or stabilizer. The terms “stability enhancing agent” or “stabilizer” are used herein to inhibit, prevent, slow down, or reduce the degradation of edaravone. More specifically, stability-enhancing agents include amino acids such as alanine, glycine, glutamate, sodium glutamate, L-arginine, lysine, L-cysteine, methionine; sodium chloride or sodium sulfate salts; sodium hydroxide, potassium hydroxide, calcium hydroxide, lactic acid, glycolic acid, succinic acid, pyruvic acid, citric acid, ethylenediaminetetraacetic acid (EDTA), non-transitioning metal ions such as zinc, magnesium and calcium or mixtures thereof, natural or synthetic gums, cellulosic derivatives such as carboxy methyl cellulose, ethyl cellulose, hydroxyethyl cellulose, hydroxyl propyl cellulose, hydroxyl propyl methylcellulose, methylcellulose, polyanionic cellulose; cyclodextrins; sugars; sugar alcohols, mannitol, glycerol or glycerin, sorbitol; monosaccharides, disaccharides or polysaccharides or combinations thereof. In yet other embodiments of the present invention, the formulation comprises stabilizer selected from group comprising of L-cysteine, DL-cysteine, N-acetylcysteine, or their salts and/or hydrates (such as L-cysteine hydrochloride monohydrate).

In some other embodiments of the present invention, the formulation of edaravone is free from sulfur containing stabilizers such as L-cysteine or its salt/hydrate, and/or sodium bisulfite, and/or sodium metabisulfite. In yet other embodiments of the present invention, the formulation of edaravone is free from sulfur containing amino acids stabilizers.

In some embodiments of the present invention, the concentration of the stabilizer ranges from 0.001% to 20%, preferably from about 0.001% to about 10%, preferably from about 0.01% to about 5%, preferably from about 0.1% to about 1%, preferably from about 0.15% to 0.5%, preferably from about 0.15% to 0.25% based on the total weight of the composition. In some embodiments, vehicle may act as stabilizer and in such cases stabilizer could be used from about 10% to about 99.99% w/w.

In some other embodiments of the present invention, the concentration of the stabilizer ranges from 0.1% to 20%, preferably from about 1% to about 20%, preferably from about 1% to about 10%, preferably from about 1% to about 5% based on the total weight of the composition.

In some embodiments of the present invention, sodium bisulfite and/or sodium metabisulfite may degrade edaravone in the formulation of the present invention. The concentration sodium bisulfite or sodium metabisulfite is less than about 5% w/v, preferably less than about 2% w/v, preferably less than about 10% w/v, from about 0% w/v to about 10% w/v, from about 0.010% w/v to about 0.50% w/v, from about 0.010% w/v to about 0.10% w/v.

### 15. Complexing Agents

According to some aspects, the liquid pharmaceutical composition of the present invention comprises one or more complexing agent(s). The term “complexing agent,” as used herein, refers to a compound or mixture of compounds that interacts with an active pharmaceutical ingredient to form a stable complex, thereby enhancing solubility, stability, palatability (taste masking) or bioavailability within the formulation. In the context of edaravone liquid pharmaceutical compositions, suitable complexing agents may include, but are not limited to, Cyclodextrins (such as α-cyclodextrin, β-cyclodextrin, γ-cyclodextrin, and their derivatives like hydroxypropyl-β-cyclodextrin, sulfobutyl ether-β-cyclodextrin), metal chelators such as ethylenediaminetetraacetic acid (EDTA), citric acid, tartaric acid, or other pharmaceutically acceptable agents capable of forming inclusion, chelation, or coordination complexes. In some embodiments, the complexing agent is selected to synergistically inhibit oxidation and hydrolysis simultaneously. The concentration of complexing agents in compositions of present invention typically ranges from about 0.01% to about 70% by weight of the total formulation, optionally from about 1% to about 40%, optionally from about 1% to about 20%, depending on the nature and binding affinity of the selected agent, and the desired solubility and/or stability and/or taste masking properties. In some embodiments of the present invention, the complexing agent may be used in a mole ratio from about 1:1 to about 15:1 (complexing agent:edaravone), preferably from about 1:1 to about 5:1, optionally from about 1:1 to about 2:1, optionally about 1:1. In some other embodiments of the present invention, the mole ratio of complexing agent:edaravone is <1:1, for example, about 0.5:1 to about 1:1, about 0.8:1 to about 1:1. In some embodiments, primary purpose of complexation is to mask the taste or improve solubility or both. In some embodiments, salts with higher aqueous solubility may be used with complexing agent to achieve the said benefits of the complexing agents.

### 16. Crystallization Inhibitors

According to some aspects, the liquid pharmaceutical composition of the present invention comprises one or more crystallization inhibitor(s). As used herein, the term “crystallization inhibitors” refers to ingredients that prevent or reduce the formation and/or growth of crystals and/or amorphous particles of edaravone within a liquid formulation, thereby stabilizing the active ingredient in a supersaturated or amorphous state and improving the clarity and homogeneity of the final product. Suitable crystallization inhibitors include, without limitation, hydrophilic polymers such as polyvinylpyrrolidone (PVP, including PVP K12, PVP K15, PVP K17, PVP K25, PVP K30, PVP K60, PVP K90, or PVP K120), polyethylene glycol (PEG 200, PEG 300, PEG 400, PEG 600, or PEG 1000), hydroxypropyl methylcellulose (HPMC), hydroxypropyl cellulose, methylcellulose, sodium carboxymethyl cellulose, polyvinyl alcohol, poloxamers, povidone, copovidone, cyclodextrins, and polysorbates (such as Polysorbate 20, Polysorbate 40, Polysorbate 60, or Polysorbate 80). In some embodiments of the present invention, organic solvents such as propylene glycol may also act as crystallization inhibitor. In some embodiments of the present invention, the concentration of crystallization inhibitor ranges from about 0.01% to about 10% based on the total weight of the composition.

### 17. Suspending Agents

According to some aspects, the liquid pharmaceutical composition of the present invention comprises one or more suspending agent(s). As used herein, the term “suspending agent” refers to a substance that helps keep the solid particle dispersed and prevent or slow down the settling to the bottom of the liquid. Suspending agents suitable for use in the liquid oral suspensions of the present invention include, cellulose derivatives such as methylcellulose, sodium carboxymethylcellulose, hydroxypropyl methyl cellulose, polyvinyl pyrrolidone (PVP, such as PVP K12, PVP K15, PVP K17, PVP K25, PVP K30, PVP K60, PVP K90, or PVP K120), alginate, guar gum, xanthan gum, carrageenan, acacia gum, tragacanth, chitosan, dextran, gelatin, polyethylene glycol, polyoxyethylene and polyoxypropylene ether.

As used herein, the term “polyhydric alcohol” refers to a compound with more than one hydroxyl group. Non-limiting examples of polyhydric alcohols that can be used are glycerin, propylene glycol, polyethylene glycol, xylitol, maltitol, sorbitol, sucrose, sucralose, glucose, and mannitol.

In some embodiments of the present invention, the concentration of polyhydric alcohol ranges from about 0.1% to 75%, preferably from about 0.1% to about 40%, preferably from about 0.1% to about 5%, preferably from about 0.1% to about 1%, preferably from about 0.1% to 0.5% based on the total weight of the composition.

In an embodiment of the present invention, the concentration of suspending agent ranges from about 0.01% to 5%, preferably from about 0.05% to about 5%, preferably from about 0.1% to about 4%, preferably from about 0.1% to about 3%, preferably from about 0.1% to about 2% based on the total weight of the composition.

### 18. Surfactants

According to some aspects, the liquid pharmaceutical composition of the present invention comprises one or more surfactant(s). As used herein, the term “surfactant” refers to any ingredient that reduces the surface tension between particle and liquid medium, allowing for better wetting and preventing agglomeration in the liquid formulation of the present invention.

In an embodiment, the liquid formulation of edaravone may contain suitable surfactants such as ionic or non-ionic surface-active agents. Suitable ionic surfactants include, but are not limited to, those containing carboxylate, sulfonate, and sulfate ions. Examples of anionic surfactants include potassium, sodium, ammonium salts of long chain alkyl sulfonates, and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate; quaternary ammonium compounds such as benzalkonium chloride, benzethonium chloride, cetrimonium bromide, stearyl dimethylbenzyl ammonium chloride, polyoxyethylene. In an embodiment, the concentration of ionic surfactant ranges from about 0.01% to 3%, preferably from about 0.05% to about 2%, preferably from about 0.05% to about 1%, preferably from about 0.05% to about 0.5%, preferably from about 0.05% to about 0.25% based on the total weight of the composition.

Suitable nonionic surfactants optionally used include, but are not limited to, glycol stearates such as ethylene glycol monostearate, propylene glycol myristate, glyceryl monostearate, glyceryl stearate, polyglyceryl-4-oleate, sorbitan acylate, sucrose acylate, polyethylene glycol dilaurate, polyethylene glycol monolaurate, polysorbates, polyoxyethylene octylphenylether, polyethylene glycol cetyl ether, polyoxyethylene tridecyl ether, polypropylene glycol butyl ether, stearoyl monoisopropanolamide, polyoxyethylene hydrogenated tallow amide, polyoxyl-ethylated castor oils (CREMOPHOR®), polyoxyethylene esters of 12-hydroxystearic acid (SOLUTOL®) and PEGylated glycerides (LABRASOL®). In another embodiment, the concentration of non-ionic surfactant ranges from about 0.02% to 2%, based on the total weight of the composition. In another embodiment, polysorbate is the non-ionic surfactant at a concentration of less than about 7% (w/w) of the total composition, less than about 6% (w/w) of the total composition, less than about 5% (w/w) of the total composition, less than about 4% (w/w) of the total composition, less than 3% (w/w) of the total composition, less than 2% (w/w) of the total composition, less than 1% (w/w) of the total composition.

### 19. Anti-Foaming Agents

According to some aspects, the liquid pharmaceutical composition of the present invention comprises one or more anti-foaming agent(s). As used herein, the term “anti-foaming agent” or “defoamers” refers to ingredients suitable to be used in the formulation of the present invention that disrupt the stability of the foam or prevent the foaming by destabilizing the thin liquid films or lamellae that form the bubble walls, causing them to rupture and collapse. Non-limiting examples of anti-foaming agents are simethicone, polydimethylsiloxane, organomodified silicones, mineral oil, vegetable oil, polypropylene glycol, fatty alcohols, aliphatic esters, triglycerides or combination thereof. In an embodiment of the present invention, the concentration of anti-foaming agent is from about 0.001% w/v to about 5% w/v, optionally from about 0.001% w/v to about 1% w/v, optionally from about 0.010% w/v to about 0.50% w/v.

### 20. Dispersing Agents

According to some aspects, the liquid pharmaceutical composition of the present invention comprises one or more dispersing agent(s). As used herein, the term “dispersing agent” refers to any substance that facilitates the uniform distribution of solid particles within a liquid medium, thereby preventing aggregation or clumping and ensuring homogeneity of the formulation of the present invention. Dispersing agents improve the stability and consistency of suspensions by enhancing the separation of individual particles and maintaining their even distribution throughout storage and dosing. Non-limiting examples of dispersing agents include polysorbates (PS, such as PS 20, PS 40, PS 60, PS 80), lecithin, polyvinylpyrrolidone (PVP, such as PVP K12, PVP K30, PVP K90), poloxamers, sodium lauryl sulfate, sodium citrate, sodium carboxymethyl cellulose, and phospholipids, as well as certain surfactants and amphiphilic polymers. In some embodiments of the present invention, the concentration of dispersing agent ranges from about 0.01% to about 5%, preferably from about 0.05% to about 2%.

### 21. Wetting Agents

According to some aspects, the liquid pharmaceutical composition of the present invention comprises one or more wetting agent(s). As used herein, the term “wetting agent” refers to any substance that facilitates the dispersion and penetration of solid particles by reducing the interfacial tension between the solid and the liquid medium in a formulation. Wetting agents play a crucial role in enhancing the ability of the liquid to spread across the surface of solids, thereby improving uniformity and consistency in pharmaceutical suspensions and other liquid formulations. Non-limiting examples of wetting agents suitable for use in the present invention include, but are not limited to, polysorbates (such as polysorbate 20, Polysorbate 40, Polysorbate 60, or Polysorbate 80), sodium lauryl sulfate, poloxamers, lecithin, polyoxyethylene sorbitan monolaurate, polyvinylpyrrolidone (PVP), sorbitan esters, cetyl alcohol, stearyl alcohol, glycerol monostearate, polyethylene glycol (such as PEG 200, 300, 400, 600, or 1000), and sodium dioctyl sulfosuccinate. In some embodiments of the present invention, the concentration of wetting agent ranges from about 0.01% to about 5% based on the total weight of the composition, preferably from about 0.05% to about 2%, and more preferably from about 0.1% to about 1%.

### 22. Emulsifying Agents

In certain embodiments, the present invention provides a liquid pharmaceutical composition comprising one or more emulsifying agents. As used herein, the term “emulsifying agent” refers to any substance capable of facilitating the formation and stabilization of emulsions by reducing interfacial tension between immiscible phases, such as oil and water. Reduction of interfacial tension enables uniform dispersion of one liquid phase within another, thereby preventing phase separation and maintaining homogeneity throughout the shelf life and administration of the composition.

The emulsifying agent may be selected from natural, semi-synthetic, or synthetic compounds. Non-limiting examples of suitable emulsifying agents include:
(a) Phospholipids, such as lecithin (egg lecithin, soy lecithin), phosphatidylcholine, phosphatidylethanolamine, phosphatidylserine, phosphatidylinositol, and hydrogenated lecithin;
(b) Polysorbates, including polysorbate 20, polysorbate 40, polysorbate 60, and polysorbate 80;
(c) Sorbitan esters, such as sorbitan monostearate, sorbitan monooleate, sorbitan monopalmitate, sorbitan tristearate, and sorbitan trioleate;
(d) Polyoxyethylene derivatives, including polyoxyethylene sorbitan monolaurate, polyoxyethylene sorbitan monostearate, and polyoxyethylene castor oil derivatives such as CREMOPHOR® EL and CREMOPHOR® RH40;
(e) Polyethylene glycol (PEG) esters and PEGylated glycerides, including PEG 300, PEG 400, PEG 600, LABRASOL®, and GELUCIRE® series;
(f) Poloxamers, such as Poloxamer 188, Poloxamer 338, and Poloxamer 407;
(g) Fatty alcohols, including cetyl alcohol, stearyl alcohol, and oleyl alcohol;
(h) Polyvinylpyrrolidone (PVP), such as PVP K12, PVP K30 and PVP K90;
(i) Monoglycerides and diglycerides, including glyceryl monostearate, glyceryl monooleate, and glyceryl dioleate;
(j) Amphiphilic polymers, such as hydroxypropyl methylcellulose (HPMC), hydroxypropyl cellulose (HPC), polyvinyl alcohol, and polyethylene oxide derivatives;
(k) Ionic surfactants, including sodium lauryl sulfate, sodium stearate, and benzalkonium chloride; and
(l) Other non-limiting examples such as PEG stearates, PEG oleates, PEG palmitates, PEG hydrogenated castor oil derivatives, PEG-40 hydrogenated castor oil, and PEG-60 hydrogenated castor oil.

In some embodiments, the emulsifying agent is present in the liquid pharmaceutical composition of the present invention in an amount ranging from about 0.001% to about 15% by weight of the total composition. In certain embodiments, the amount ranges from about 0.01% to about 10%, optionally from about 0.05% to about 5%, and in preferred embodiments from about 0.1% to about 2%. For formulations containing a high proportion of oil phase, the concentration of emulsifying agent may be increased up to about 20% to achieve desired stability.

The concentration of emulsifying agent may be adjusted based on formulation parameters, including the proportion of oil phase, the type of emulsifier employed, and the target globule size of the emulsion. For example, low-oil formulations (oil phase less than about 10%) may utilize emulsifier concentrations between about 0.01% and about 1%; medium-oil formulations (oil phase between about 10% and about 30%) may utilize emulsifier concentrations between about 0.5% and about 5%; and high-oil formulations (oil phase greater than about 30%) may utilize emulsifier concentrations between about 2% and about 10%, or higher for specialized systems. Optimization of these parameters ensures physical stability and performance, maintaining uniformity and preventing phase separation throughout the intended shelf life of the composition.

### 23. Film Forming Agents

Film forming agents suitable for use in liquid-filled solid dosage forms, such as soft gelatin or hard gelatin capsules, include a variety of polymers that provide structural integrity, encapsulation efficiency, and controlled release properties. For capsule shells, commonly used film formers are gelatin (from bovine or porcine sources), hydroxypropyl methylcellulose (HPMC), pullulan, and starch derivatives. Plasticizers such as glycerin, sorbitol, or polyethylene glycol may be incorporated to impart desired flexibility and reduce brittleness. For capsule coatings, additional film formers may include polyvinyl alcohol, ethylcellulose, methacrylic acid copolymers (such as Eudragit® types), and shellac. These agents can be selected and combined based on the required dissolution profile, stability, and compatibility with the fill formulation. Functional coatings may further include enteric or sustained-release polymers to modulate drug release in the gastrointestinal tract.

In certain embodiments, the capsule material may be modified with coloring agents, opacifiers (such as titanium dioxide), or flavors to enhance product appearance and patient acceptability.

In embodiments requiring delayed release, the liquid filled solid dosage form is coated with one or more enteric polymers selected for their pH-dependent solubility characteristics. Suitable enteric coating polymers for use in such formulations include, but are not limited to, cellulose-based derivatives such as cellulose acetate phthalate (CAP), hydroxypropyl methylcellulose phthalate (HPMCP), hydroxypropyl methylcellulose acetate succinate (HPMCAS), cellulose acetate trimellitate (CAT), and certain derivatives of carboxymethylcellulose coating polymers. Other suitable polymers include vinyl polymers, for instance, polyvinyl acetate phthalate (PVAP), and a range of acrylic copolymers. The acrylic copolymers may comprise various methacrylic acid copolymers, including those commercially available under the Eudragit® trade name (e.g., Eudragit® L 100-55, Eudragit® L, Eudragit® S, Eudragit® FS), as well as poly(methacrylic acid-co-methyl methacrylate). Furthermore, naturally derived polymers may be employed for the enteric coating, such as the purified resin shellac, zein, and polysaccharides including sodium alginate, pectin, guar gum, and chitosan. These polymers may be utilized individually or as combinations thereof. The selection of the specific polymer or polymer blend is based on its ability to substantially resist dissolution in the acidic gastric environment while dissolving at a desired pH within the intestine to enable targeted drug release, thereby protecting acid-sensitive drugs, preventing gastric irritation, and improving therapeutic outcomes. The quantity of delayed release polymers may range from about 1% w/w to about 50% w/w, optionally from about 5% w/w to about 25% w/w.

### Method of Preparation

In an embodiment, the present invention provides a process for preparing edaravone solution suitable for oral administration comprising the steps of: (1) adding vehicle to manufacturing vessel; (2) adding one or more optional ingredients one-by-one with mixing until dissolved; (3) adding edaravone with mixing until dissolved; (4) optionally adjusting pH between about 2 and about 6, preferably between about 3 and about 6; (5) optionally filtering the solution using suitable filter such as form about 0.2 μm to about 20 μm pore size; (6) filling the edaravone solution in suitable primary packaging containers; and (7) closing the containers. Such containers may be further packaged in carton, optionally with delivery devices such as syringe or measuring cup or measuring spoon.

In some embodiments, the containers are purged with inert gas such as nitrogen or argon before closing with closure. In some other embodiments, the liquid vehicles are sparged with inert gas bubbles to reduce dissolved oxygen. In yet another embodiment, the antioxidant is added and dissolved prior to adding edaravone in the vehicle.

In an embodiment, the present invention provides a process for preparing edaravone solution suitable for intranasal administration in the form of unit dose or bi-dose spray comprising the steps of: (1) adding vehicle to manufacturing vessel; (2) adding one or more optional ingredients one-by-one with mixing until dissolved; (3) adding edaravone with mixing until dissolved; (4) optionally adjusting pH between about 3 and about 6; (5) optionally filtering the solution using suitable filter such as 0.2 μm filter; (6) filling the edaravone solution in vials, closed with polymeric stopper and assemble with unit or bi-dose nasal spray device; and (7) optionally packaging such device further into tray or carton. In some embodiments, the formulations may be preservative free, wherein the containers are pre-sterilized and the formulation is filled aseptically.

In an embodiment, the present invention provides a process for preparing edaravone solution suitable for intranasal administration in the form of multiple dose spray comprising the steps of: (1) adding vehicle to manufacturing vessel; (2) adding one or more optional ingredients one-by-one with mixing until dissolved; (3) adding edaravone with mixing until dissolved; (4) optionally adjusting pH between about 3 and about 6; (5) optionally filtering the solution using suitable filter such as 0.2 μm filter; (6) filling the edaravone solution in containers and closing with closure assembled with metering spray device; and (7) optionally packaging such device further into tray or carton. In some embodiments of the present invention, the product is sterilized by aseptic filtration without terminal sterilization.

In an embodiment, the present invention provides a process for preparing edaravone solution suitable for injectable route of administration comprising the steps of: (1) adding vehicle to manufacturing vessel; (2) adding optional ingredients one-by-one with mixing until dissolved; (3) adding edaravone with mixing until dissolved; (4) optionally adjusting pH between about 3 and about 6; (5) sterilizing the solution using sterile grade 0.2 μm filter; (6) aseptically filling the edaravone solution in suitable primary package such as cartridge, vial or pre-fillable syringe; and (7) closing the primary container with appropriate closure with optional nitrogen overlay.

In some embodiments of the present invention, the stable liquid formulation of edaravone is suitable for terminal sterilization using methods such as moist heat sterilization at about 121° C. for about 5 to about 20 minutes.

In an embodiment, the present invention provides a process for preparing edaravone suspension suitable for intranasal administration comprising the steps of: (1) adding vehicle to manufacturing vessel; (2) adding one or more ingredients one-by-one with mixing until dissolved or suspended; (3) suspending edaravone with mixing until homogenous suspension obtained; (4) optionally adjusting pH between about 3 and about 6; (5) filling the edaravone suspension in containers and closing with closure assembled with metering spray device, with optional nitrogen overlay; and (6) optionally packaging such device further into tray or carton.

In an embodiment, the present invention provides a process for preparing a delayed-release soft gelatin capsule containing Edaravone. The method comprises preparing an Edaravone-containing fill formulation by dissolving, suspending, or solubilizing Edaravone in a pharmaceutically acceptable liquid vehicle, which may include hydrophilic solvents (e.g., polyethylene glycol, propylene glycol) or lipophilic solvents (e.g., medium chain triglycerides, sesame oil), optionally admixed with excipients such as antioxidants, surfactants, or stabilizers to achieve the desired viscosity and homogeneity. The process further comprises preparing a shell formulation comprising gelatin or a non-animal polymer and a plasticizer, wherein an enteric release agent (such as pectin, alginic acid, or acrylic polymers) is optionally incorporated directly into the shell mass to impart inherent gastro-resistance. The prepared fill formulation is encapsulated into the shell formulation using rotary die encapsulation or similar techniques, followed by drying under controlled temperature and humidity to achieve shell integrity. Finally, an enteric coating comprising pH-sensitive polymers (e.g., methacrylic acid copolymers, hydroxypropyl methylcellulose phthalate, or cellulose acetate phthalate) may optionally be applied to the dried capsule shell. The resulting soft gelatin capsules effectively provide delayed release, protecting the Edaravone from degradation in the acidic environment of the stomach and promoting targeted release in the intestine.

### Packaging

In an embodiment, the edaravone liquid formulation of the present invention is filled into a suitable pharmaceutically acceptable container selected from the group comprising of bottles, bags, sprayers, ampules (plastic or glass), blisters, sachets, syringes, cartridges, and vials.

In another embodiment, the edaravone liquid formulation of the present invention is filled into a pharmaceutically acceptable container suitable for single or multiple-dose delivery for oral, intranasal or injectable administration.

In an embodiment, the edaravone liquid formulation of the present invention is filled into a suitable pharmaceutically acceptable container that reasonably protects the composition from air and light that otherwise may degrade edaravone or any vehicle or excipient within the container.

In an embodiment, the edaravone liquid formulation of the present invention is administered via oral route to the patient as a fluid stream, drop(s), droplet(s), spray, or combination thereof.

In an embodiment, the pharmaceutically acceptable container for the edaravone liquid formulation is a bottle, wherein the bottle is selected from a group comprising of glass bottles or plastic bottles, wherein glass bottle is selected from a group consisting of Type I, II, and III borosilicate glass bottles, wherein the glass bottle may be amber color glass bottle or clear glass bottle.

In another embodiment, the pharmaceutically acceptable container for the edaravone liquid formulations is a bottle, wherein the bottle is selected from a group comprising of high-density polyethylene (HDPE) bottle, polyethylene terephthalate (PET) and polypropylene (PP), wherein the plastic bottle may be amber color, white opaque or translucent plastic bottle or any other color.

In an embodiment, the ready-to-use liquid formulation of edaravone is packaged in flexible polymeric bag contained in secondary outer packaging.

In some embodiments, the glass or polymeric containers for the liquid edaravone formulations may be provided with a fill volume in the range from about 0.05 mL to about 500 mL, optionally from about 1 mL to about 250 mL, optionally from about 1 mL to about 10 mL, optionally from about 10 mL to about 50 mL, optionally from about 50 mL to about 100 mL, optionally from about 100 mL to about 150 mL, optionally from about 150 mL to about 200 mL, optionally from about 200 mL to about 250 mL, optionally from about 250 mL to about 300 mL, optionally from about 300 mL to about 500 mL. In some other embodiments, the fill volume of edaravone liquid formulation is about 1 mL, about 2 mL, about 3 mL, about 5 mL, about 10 mL, about 12 mL, about 15 mL, about 20 mL, about 25 mL, about 30 mL, about 40 mL, about 50 mL, about 60 mL, about 80 mL, about 90 mL, about 100 mL, about 115 mL, about 125 mL, about 150 mL, about 180 mL, about 200 mL, about 210 mL, about 240 mL, about 250 mL, about 270 mL, about 300 mL, about 320 mL, about 350 mL, about 400 mL, about 450 mL or up to about 500 mL fill volumes. In some other embodiments, the volume filled in primary container is about 0.5 ounce, about 1 ounce, about 2 ounce, about 3 ounce, about 4 ounce, about 5 ounce, about 6 ounce, about 7 ounce, about 8 ounce, about 9 ounce, about 10 ounce, about 11 ounce, about 12 ounce, about 14 ounce, about 15 ounce, or up to about 16 ounce. Any volume within this range is within the scope of the present invention.

In some embodiments of the present invention, the liquid formulation filled solid dosage forms may be provided in glass or plastic bottles with a capacity or size of about 1 ounce to about 20 ounce, about 2 ounce, about 3 ounce, about 4 ounce, about 5 ounce, about 6 ounce, about 7 ounce, about 8 ounce, about 9 ounce, about 10 ounce, about 11 ounce, about 12 ounce, about 14 ounce, about 15 ounce, or up to about 20 ounce.

In some embodiments of the present invention, the liquid formulation filled solid dosage forms may be provided in glass or plastic bottles in pack of about 10 count, about 15 count, about 20 count, about 25 count, about 30 count, about 35 count, about 40 count, about 45 count, about 50 count, about 60 count, about 70 count, about 80 count, about 90 count, about 100 count, about 120 count, about, 150 count, about 200 count, about 250 count, or up to about 300 count per bottle.

In some embodiments of the present invention, the liquid formulation filled solid dosage forms may be provided in blister pack (such as aluminum-aluminum, or aluminum-plastic or paper) in pack size of about 10 count, about 15 count, about 20 count, about 25 count, about 30 count, about 35 count, about 40 count, about 45 count, about 50 count, about 60 count, about 70 count, about 80 count, about 90 count, about 100 count, about 120 count, about, 150 count, about 200 count, about 250 count, or up to about 300 count per blister.

In some embodiments, the liquid formulation filled solid dosage forms may be filled in containers along with desiccant or oxygen scavengers to protect from moisture and oxygen.

In an embodiment, the pharmaceutical composition of the present invention may be further packaged as a convenience kit comprising a bottle with a child-resistant cap, adapter or flow restrictor, and dosing syringe.

In another embodiment, the liquid edaravone formulations may be packaged in a metered device that protects the composition from air, light, and microbial contamination during storage and use.

In some embodiments of the present invention, the liquid formulation of edaravone may be filled and packaged into unit-dose, bi-dose or multiple dose nasal spray devices manufactured by, without limitation, Aptar Pharma, Nemera, Bona Pharma or similar other manufacturers and available and/or approved in the market. In some embodiments, the edaravone liquid formulation of the present invention are filled and assembled into spray devices with advanced characteristics such as preservative free pump, springless pump, recyclable pump, etc.

### Formulation Characteristics

In some embodiments of the present invention, the pH of the Edaravone liquid formulation is between about 2 and about 6, optionally between about 2.5 and about 5.5, optionally between about 3 and about 5.5, optionally between about 3.5 and about 4.5, optionally between about 3.0 and about 6.0, optionally between about 4.0 and about 6.0, or optionally between about 2.0 and about 4.0. Specific pH values may be selected from about 2.5, about 3.0, about 3.5, about 3.8, about 4.2, about 4.5, about 5.0, about 5.5, or about 6.0. In one example, the pH of the formulation is maintained from about 3.0 to about 4.5 to maximize chemical stability against oxidation. In another example, the pH of the formulation is adjusted to about 4.5 to about 6.0 to improve physiological compatibility. In some embodiments suitable for oral administration (e.g., solution or syrup), the formulation may have a pH between about 2.5 and about 4.5. In some other embodiments suitable for intranasal administration (e.g., spray or drops), the formulation may have a pH between about 4.0 and about 6.0 to minimize mucosal irritation while maintaining drug solubility. In further embodiments comprising a suspension or complexed edaravone, the pH may be buffered between about 4.0 and about 5.5.

In some embodiments, the edaravone liquid formulation of the present invention has water like viscosity such as about 1 cP. In some other embodiments, the viscosity of the formulation is higher than 1 cP, such as about 1 to 3 cP, about 3 to 10 cP, about 10 to 20 cP, about 20 to 30 cP, about 30 to 50 cP, about 50 to 100 cP, about 100 to about 1000 cP, about 1000 to about 1750 cP, about 1500 to about 3000 cP, or about 3000 to about 5000. In some other embodiments, the viscosity of the formulation is from about 1 to 50 cP, preferably from about 5 to 50 cP. In yet other embodiments, the viscosity of the formulation is from about 10 to 500 cP, preferably from about 50 to 250 cP. Higher than water viscosity may help increase residence time of the edaravone formulation on nasal mucosa for better absorption. Additionally, viscous liquid formulation of the present invention may ease the administration in patients with dysphagia (such as ALS, or aged patients with difficulty in swallowing). The viscosity range provided here is when tested using capillary viscometer, such as U tube Ostwald capillary viscometer with ID of about 0.5 mm or about 1 mm, using water as reference. Viscosity of the edaravone liquid formulation of the present invention may depend on the route of administration and end use needs. For instance, injectable formulations of the present invention may have water like viscosity for intravenous use, while sustained release formulation suitable for depot injection may have viscosity like gel (such as from about 3000 cP to about 100,000 cP). Oral solution may have water like viscosity or even higher up to about 5000 cP for slower drug release to match the pharmacokinetic and AUC with other formulation such as tablet or suspension. Nasal spray formulation may have slightly higher viscosity from about 5 cP to about 100 cP, optionally from about 5 cP to about 50 cP for higher residence time on nasal mucosa. Viscosity of edaravone liquid formulation of present invention to be used for ALS patients may have viscosity from about 10 cP to about 2000 cP, optionally from about 50 to about 100 cP, optionally from about 100 to about 400 cP, optionally from about 400 to 800 cP to avoid aspiration, depending on patients' condition and needs.

In some embodiments of the present invention, the liquid formulation of edaravone suitable for oral administration has viscosity of which an IDDSI (International Dysphagia Diet Standardization Initiative) level is classified in a range of 0-3, optionally 1-3, optionally 2 when viscosity is measured using Framework Flow Test.

In some embodiments, the edaravone liquid formulation of the present invention has osmolality from about 100 mOsmol/kg to about 2000 mOsmol/kg, from about 200 mOsmol/Kg to about 1000 mOsmol/Kg, from about 250 mOsmol/Kg to about 800 mOsmol/Kg, from about 250 mOsmol/Kg to about 550 mOsmol/Kg, from about 250 mOsomol/Kg to about 350 mOsoml/Kg. In yet other embodiments, the osmolality of the edaravone liquid formulation is about 280 to 320 mOsmol/Kg. In one example, the osmolality of the edaravone liquid formulation is about 270 to about 330 mOsmol/Kg.

In some embodiments of the present invention, the nasal spray delivers, upon actuation, edaravone formulation in the form of spray, wherein less than 10% (D10) of the droplets have a size smaller than about 10 μm, optionally smaller than about 5 μm, and/or at least 50% (D50) of the droplets have a size from about 10 μm to 150 μm, preferably from about 40 μm to about 80 μm, and/or at least 90% (D90) of the droplet have a size smaller than 300 μm, preferably smaller than 150 μm, more preferably smaller than 120 μm.

In some embodiments of the present invention, the nasal spray delivers, upon actuation, edaravone formulation in the form of spray, wherein plume angle of the spray is from about 25° to about 75°, preferably from about 30° to about 60°.

In some embodiments, the particle size distribution (PSD) of edaravone in pharmaceutical suspension formulation of the present invention comprises at least 10% of particles smaller than about 30 microns, and/or at least 50% of particles smaller than about 70 microns, and/or at least 90% of particles smaller than about 150 microns.

In some embodiments of the present invention, the particle size distribution (PSD) of edaravone in pharmaceutical suspension formulation of the present invention comprises at least 10% of particles smaller than about 10 microns, and/or at least 50% of particles smaller than about 50 microns, and/or at least 90% of particles smaller than about 100 microns.

In some embodiments of the present invention, the particle size distribution (PSD) of edaravone in pharmaceutical suspension formulation of the present invention comprises at least 10% of particles smaller than about 10 microns, and/or at least 50% of particles smaller than about 25 microns, and/or at least 90% of particles smaller than about 50 microns.

In some embodiments of the present invention, the particle size distribution (PSD) of edaravone in pharmaceutical suspension formulation of the present invention comprises at least 10% of particles smaller than about 2 microns, and/or at least 50% of particles smaller than about 5 microns, and/or at least 90% of particles smaller than about 10 microns.

In some embodiments of the present invention, the particle size of edaravone in suspension formulation comprises at least 90% of particles smaller than about 300 microns, preferably smaller than about 200 microns, more preferably smaller than about 100 microns, more preferably smaller than about 90 microns, more preferably smaller than about 80 microns, more preferably smaller than about 70 microns, more preferably smaller than about 60 microns, more preferably smaller than about 50 microns, more preferably smaller than about 45 microns, more preferably smaller than about 40 microns, more preferably smaller than about 35 microns, more preferably smaller than about 33 microns, more preferably smaller than about 30 microns, more preferably smaller than about 28 microns, more preferably smaller than about 25 microns, more preferably smaller than about 23 microns, more preferably smaller than about 20 microns, more preferably smaller than about 18 microns, more preferably smaller than about 16 microns, more preferably smaller than about 15 microns, more preferably smaller than about 14 microns, more preferably smaller than about 13 microns, more preferably smaller than about 12 microns, more preferably smaller than about 11 microns, more preferably smaller than about 10 microns, more preferably smaller than about 9 microns, more preferably smaller than about 8 microns, more preferably smaller than about 7 microns, more preferably smaller than about 6 microns, or more preferably smaller than about 5 microns. In some other embodiments, the particle size of edaravone in suspension formulation comprises at least 90% of particles are within the range from about 5 microns to about 10 microns.

In some embodiments of the present invention, the edaravone solution suitable for intranasal administration is filled in unit-dose or bi-dose or multiple dose nasal spray device which forms plume of droplets when actuated at force between about 5N and about 50N, optionally between about 10N and about 50N.

In some embodiments of the present invention, the liquid pharmaceutical composition is encapsulated within delayed release capsules such as soft gelatin or hard gelatin capsule. Without wishing to be bound by any theoretical principles, the liquid filled-soft gelatin capsule may dissolve faster than delayed release dosage form with solid core such as enteric coated tablet, enteric coated pellets, enteric coated tablets or pellets or particulates filled in immediate release capsule or similar delayed release formulations with solid core. In some embodiments, the dissolution time of the delayed release capsule in phosphate buffer pH 6.8 is less than about 60 minutes, optionally less than about 30 minutes, optionally less than about 25 minutes, optionally less than about 15 minutes, optionally less than about 10 minutes, optionally less than about 5 minutes, optionally between about 2 to 5 minutes, optionally between about 5 to 10 minutes, optionally between about 10 to 30 minutes when tested using appropriate dissolution method given in pharmacopeia such as USP.

### Stability and Impurity Embodiments

In some embodiments of the present invention, the liquid pharmaceutical formulation of edaravone is stable for at least 1 week, preferably for at least 2 weeks, preferably for at least 3 weeks, preferably for at least about 1 month, preferably for at least about 2 months, preferably for at least about 3 months, preferably for at least about 4 months, preferably for at least about 5 months, preferably for at least about 6 months, preferably for at least about 8 months, preferably for at least about 10 months, preferably for at least about 12 months, preferably for at least about 15 months, preferably for at least about 18 months, preferably for at least about 21 months, preferably for at least about 24 months, preferably for at least about 27 months, preferably for at least about 30 months, preferably for at least about 33 months, preferably for at least about 36 months at room temperature (about 15-20° C.).

In some embodiments of the present invention, the liquid pharmaceutical formulation of edaravone is stable for at least 1 week, preferably for at least 2 weeks, preferably for at least 3 weeks, preferably for at least about 1 month, preferably for at least about 2 months, preferably for at least about 3 months, preferably for at least about 4 months, preferably for at least about 5 months, preferably for at least about 6 months, preferably for at least about 8 months, preferably for at least about 10 months, preferably for at least about 12 months, preferably for at least about 15 months, preferably for at least about 18 months, preferably for at least about 21 months, preferably for at least about 24 months, preferably for at least about 27 months, preferably for at least about 30 months, preferably for at least about 33 months, preferably for at least about 36 months at refrigeration condition (2-8° C.).

According to some aspects, the pharmaceutical composition of the present invention may have an initial total impurity concentration of no more than about 5%, more preferably no more than about 4.5%, more preferably no more than about 4%, more preferably no more than about 3.5%, more preferably no more than about 3%, more preferably no more than about 2.5%, more preferably no more than about 2%, more preferably no more than about 1.5%, more preferably no more than about 1%, and most preferably no more than about 0.5%.

According to some aspects, the pharmaceutical composition of the present invention may have an initial total impurity concentration of no more than about 1%, more preferably no more than about 0.99%, more preferably no more than about 0.98%, more preferably no more than about 0.97%, more preferably no more than about 0.96%, more preferably no more than about 0.95%, more preferably no more than about 0.94%, more preferably no more than about 0.93%, more preferably no more than about 0.92%, more preferably no more than about 0.91%, more preferably no more than about 0.90%, more preferably no more than about 0.89%, more preferably no more than about 0.88%, more preferably no more than about 0.87%, more preferably no more than about 0.86%, more preferably no more than about 0.85%, more preferably no more than about 0.84%, more preferably no more than about 0.83%, more preferably no more than about 0.82%, more preferably no more than about 0.81%, more preferably no more than about 0.80%, more preferably no more than about 0.79%, more preferably no more than about 0.78%, more preferably no more than about 0.77%, more preferably no more than about 0.76%, more preferably no more than about 0.75%, more preferably no more than about 0.74%, more preferably no more than about 0.73%, more preferably no more than about 0.72%, more preferably no more than about 0.71%, more preferably no more than about 0.97%, more preferably no more than about 0.69%, more preferably no more than about 0.68%, more preferably no more than about 0.67%, more preferably no more than about 0.66%, more preferably no more than about 0.65%, more preferably no more than about 0.64%, more preferably no more than about 0.63%, more preferably no more than about 0.62%, more preferably no more than about 0.61%, more preferably no more than about 0.60%, more preferably no more than about 0.59%, more preferably no more than about 0.58%, more preferably no more than about 0.57%, more preferably no more than about 0.56%, more preferably no more than about 0.55%, more preferably no more than about 0.54%, more preferably no more than about 0.53%, more preferably no more than about 0.52%, more preferably no more than about 0.51%, more preferably no more than about 0.50%, more preferably no more than about 0.49%, more preferably no more than about 0.48%, more preferably no more than about 0.47%, more preferably no more than about 0.46%, more preferably no more than about 0.45%, more preferably no more than about 0.44%, more preferably no more than about 0.43%, more preferably no more than about 0.42%, more preferably no more than about 0.41%, more preferably no more than about 0.40%, more preferably no more than about 0.39%, more preferably no more than about 0.38%, more preferably no more than about 0.37%, more preferably no more than about 0.36%, more preferably no more than about 0.35%, more preferably no more than about 0.34%, more preferably no more than about 0.33%, more preferably no more than about 0.32%, more preferably no more than about 0.31%, more preferably no more than about 0.30%, more preferably no more than about 0.29%, more preferably no more than about 0.28%, more preferably no more than about 0.27%, more preferably no more than about 0.26%, more preferably no more than about 0.25%, more preferably no more than about 0.24%, more preferably no more than about 0.23%, more preferably no more than about 0.22%, more preferably no more than about 0.21%, more preferably no more than about 0.20%, more preferably no more than about 0.19%, more preferably no more than about 0.18%, more preferably no more than about 0.17%, more preferably no more than about 0.16%, more preferably no more than about 0.15%, more preferably no more than about 0.14%, more preferably no more than about 0.13%, more preferably no more than about 0.12%, more preferably no more than about 0.11%, and most preferably no more than about 0.10%.

According to some aspects, the pharmaceutical composition of the present invention may have a total impurity concentration after a certain period of shelf life of no more than about 5%, more preferably no more than about 4.5%, more preferably no more than about 4%, more preferably no more than about 3.5%, more preferably no more than about 3%, more preferably no more than about 2.5%, more preferably no more than about 2%, more preferably no more than about 1.5%, more preferably no more than about 1%, and most preferably no more than about 0.5%.

According to some aspects, the pharmaceutical composition of the present invention may have a total impurity concentration after a certain period of shelf life of no more than about 1%, more preferably no more than about 0.99%, more preferably no more than about 0.98%, more preferably no more than about 0.97%, more preferably no more than about 0.96%, more preferably no more than about 0.95%, more preferably no more than about 0.94%, more preferably no more than about 0.93%, more preferably no more than about 0.92%, more preferably no more than about 0.91%, more preferably no more than about 0.90%, more preferably no more than about 0.89%, more preferably no more than about 0.88%, more preferably no more than about 0.87%, more preferably no more than about 0.86%, more preferably no more than about 0.85%, more preferably no more than about 0.84%, more preferably no more than about 0.83%, more preferably no more than about 0.82%, more preferably no more than about 0.81%, more preferably no more than about 0.80%, more preferably no more than about 0.79%, more preferably no more than about 0.78%, more preferably no more than about 0.77%, more preferably no more than about 0.76%, more preferably no more than about 0.75%, more preferably no more than about 0.74%, more preferably no more than about 0.73%, more preferably no more than about 0.72%, more preferably no more than about 0.71%, more preferably no more than about 0.97%, more preferably no more than about 0.69%, more preferably no more than about 0.68%, more preferably no more than about 0.67%, more preferably no more than about 0.66%, more preferably no more than about 0.65%, more preferably no more than about 0.64%, more preferably no more than about 0.63%, more preferably no more than about 0.62%, more preferably no more than about 0.61%, more preferably no more than about 0.60%, more preferably no more than about 0.59%, more preferably no more than about 0.58%, more preferably no more than about 0.57%, more preferably no more than about 0.56%, more preferably no more than about 0.55%, more preferably no more than about 0.54%, more preferably no more than about 0.53%, more preferably no more than about 0.52%, more preferably no more than about 0.51%, more preferably no more than about 0.50%, more preferably no more than about 0.49%, more preferably no more than about 0.48%, more preferably no more than about 0.47%, more preferably no more than about 0.46%, more preferably no more than about 0.45%, more preferably no more than about 0.44%, more preferably no more than about 0.43%, more preferably no more than about 0.42%, more preferably no more than about 0.41%, more preferably no more than about 0.40%, more preferably no more than about 0.39%, more preferably no more than about 0.38%, more preferably no more than about 0.37%, more preferably no more than about 0.36%, more preferably no more than about 0.35%, more preferably no more than about 0.34%, more preferably no more than about 0.33%, more preferably no more than about 0.32%, more preferably no more than about 0.31%, more preferably no more than about 0.30%, more preferably no more than about 0.29%, more preferably no more than about 0.28%, more preferably no more than about 0.27%, more preferably no more than about 0.26%, more preferably no more than about 0.25%, more preferably no more than about 0.24%, more preferably no more than about 0.23%, more preferably no more than about 0.22%, more preferably no more than about 0.21%, more preferably no more than about 0.20%, more preferably no more than about 0.19%, more preferably no more than about 0.18%, more preferably no more than about 0.17%, more preferably no more than about 0.16%, more preferably no more than about 0.15%, more preferably no more than about 0.14%, more preferably no more than about 0.13%, more preferably no more than about 0.12%, more preferably no more than about 0.11%, and most preferably no more than about 0.10%. The formulations of the present invention, when stored for at least three months at room temperature, the formulation has: at least about 99% pure edaravone and less than about 1% of total degradant impurities.

According to some aspects, the pharmaceutical composition of the present invention may have an initial maximum single impurity concentration of no more than about 5%, more preferably no more than about 4.5%, more preferably no more than about 4%, more preferably no more than about 3.5%, more preferably no more than about 3%, more preferably no more than about 2.5%, more preferably no more than about 2%, more preferably no more than about 1.5%, more preferably no more than about 1%. In a most preferred aspect, the formulation contains less than 0.5% maximum single impurity.

According to some aspects, the pharmaceutical composition of the present invention may have an initial maximum single impurity concentration of no more than about 1%, more preferably no more than about 0.99%, more preferably no more than about 0.98%, more preferably no more than about 0.97%, more preferably no more than about 0.96%, more preferably no more than about 0.95%, more preferably no more than about 0.94%, more preferably no more than about 0.93%, more preferably no more than about 0.92%, more preferably no more than about 0.91%, more preferably no more than about 0.90%, more preferably no more than about 0.89%, more preferably no more than about 0.88%, more preferably no more than about 0.87%, more preferably no more than about 0.86%, more preferably no more than about 0.85%, more preferably no more than about 0.84%, more preferably no more than about 0.83%, more preferably no more than about 0.82%, more preferably no more than about 0.81%, more preferably no more than about 0.80%, more preferably no more than about 0.79%, more preferably no more than about 0.78%, more preferably no more than about 0.77%, more preferably no more than about 0.76%, more preferably no more than about 0.75%, more preferably no more than about 0.74%, more preferably no more than about 0.73%, more preferably no more than about 0.72%, more preferably no more than about 0.71%, more preferably no more than about 0.97%, more preferably no more than about 0.69%, more preferably no more than about 0.68%, more preferably no more than about 0.67%, more preferably no more than about 0.66%, more preferably no more than about 0.65%, more preferably no more than about 0.64%, more preferably no more than about 0.63%, more preferably no more than about 0.62%, more preferably no more than about 0.61%, more preferably no more than about 0.60%, more preferably no more than about 0.59%, more preferably no more than about 0.58%, more preferably no more than about 0.57%, more preferably no more than about 0.56%, more preferably no more than about 0.55%, more preferably no more than about 0.54%, more preferably no more than about 0.53%, more preferably no more than about 0.52%, more preferably no more than about 0.51%, more preferably no more than about 0.50%, more preferably no more than about 0.49%, more preferably no more than about 0.48%, more preferably no more than about 0.47%, more preferably no more than about 0.46%, more preferably no more than about 0.45%, more preferably no more than about 0.44%, more preferably no more than about 0.43%, more preferably no more than about 0.42%, more preferably no more than about 0.41%, more preferably no more than about 0.40%, more preferably no more than about 0.39%, more preferably no more than about 0.38%, more preferably no more than about 0.37%, more preferably no more than about 0.36%, more preferably no more than about 0.35%, more preferably no more than about 0.34%, more preferably no more than about 0.33%, more preferably no more than about 0.32%, more preferably no more than about 0.31%, more preferably no more than about 0.30%, more preferably no more than about 0.29%, more preferably no more than about 0.28%, more preferably no more than about 0.27%, more preferably no more than about 0.26%, more preferably no more than about 0.25%, more preferably no more than about 0.24%, more preferably no more than about 0.23%, more preferably no more than about 0.22%, more preferably no more than about 0.21%, more preferably no more than about 0.20%, more preferably no more than about 0.19%, more preferably no more than about 0.18%, more preferably no more than about 0.17%, more preferably no more than about 0.16%, more preferably no more than about 0.15%, more preferably no more than about 0.14%, more preferably no more than about 0.13%, more preferably no more than about 0.12%, more preferably no more than about 0.11%, more preferably no more than about 0.09%, more preferably no more than about 0.08%, more preferably no more than about 0.07%, more preferably no more than about 0.06%, more preferably no more than about 0.05%, more preferably no more than about 0.04%, more preferably no more than about 0.03%, more preferably no more than about 0.02%, and most preferably no more than about 0.01%.

According to some aspects, the pharmaceutical composition of the present invention may have maximum single impurity concentration after a certain period of shelf life of no more than about 5%, more preferably no more than about 4.5%, more preferably no more than about 4%, more preferably no more than about 3.5%, more preferably no more than about 3%, more preferably no more than about 2.5%, more preferably no more than about 2%, more preferably no more than about 1.5%, more preferably no more than about 1%, and most preferably no more than about 0.5%.

According to some aspects, the pharmaceutical composition of the present invention may have maximum single impurity concentration after a certain period of shelf life of no more than about 1%, more preferably no more than about 0.99%, more preferably no more than about 0.98%, more preferably no more than about 0.97%, more preferably no more than about 0.96%, more preferably no more than about 0.95%, more preferably no more than about 0.94%, more preferably no more than about 0.93%, more preferably no more than about 0.92%, more preferably no more than about 0.91%, more preferably no more than about 0.90%, more preferably no more than about 0.89%, more preferably no more than about 0.88%, more preferably no more than about 0.87%, more preferably no more than about 0.86%, more preferably no more than about 0.85%, more preferably no more than about 0.84%, more preferably no more than about 0.83%, more preferably no more than about 0.82%, more preferably no more than about 0.81%, more preferably no more than about 0.80%, more preferably no more than about 0.79%, more preferably no more than about 0.78%, more preferably no more than about 0.77%, more preferably no more than about 0.76%, more preferably no more than about 0.75%, more preferably no more than about 0.74%, more preferably no more than about 0.73%, more preferably no more than about 0.72%, more preferably no more than about 0.71%, more preferably no more than about 0.97%, more preferably no more than about 0.69%, more preferably no more than about 0.68%, more preferably no more than about 0.67%, more preferably no more than about 0.66%, more preferably no more than about 0.65%, more preferably no more than about 0.64%, more preferably no more than about 0.63%, more preferably no more than about 0.62%, more preferably no more than about 0.61%, more preferably no more than about 0.60%, more preferably no more than about 0.59%, more preferably no more than about 0.58%, more preferably no more than about 0.57%, more preferably no more than about 0.56%, more preferably no more than about 0.55%, more preferably no more than about 0.54%, more preferably no more than about 0.53%, more preferably no more than about 0.52%, more preferably no more than about 0.51%, more preferably no more than about 0.50%, more preferably no more than about 0.49%, more preferably no more than about 0.48%, more preferably no more than about 0.47%, more preferably no more than about 0.46%, more preferably no more than about 0.45%, more preferably no more than about 0.44%, more preferably no more than about 0.43%, more preferably no more than about 0.42%, more preferably no more than about 0.41%, more preferably no more than about 0.40%, more preferably no more than about 0.39%, more preferably no more than about 0.38%, more preferably no more than about 0.37%, more preferably no more than about 0.36%, more preferably no more than about 0.35%, more preferably no more than about 0.34%, more preferably no more than about 0.33%, more preferably no more than about 0.32%, more preferably no more than about 0.31%, more preferably no more than about 0.30%, more preferably no more than about 0.29%, more preferably no more than about 0.28%, more preferably no more than about 0.27%, more preferably no more than about 0.26%, more preferably no more than about 0.25%, more preferably no more than about 0.24%, more preferably no more than about 0.23%, more preferably no more than about 0.22%, more preferably no more than about 0.21%, more preferably no more than about 0.20%, more preferably no more than about 0.19%, more preferably no more than about 0.18%, more preferably no more than about 0.17%, more preferably no more than about 0.16%, more preferably no more than about 0.15%, more preferably no more than about 0.14%, more preferably no more than about 0.13%, more preferably no more than about 0.12%, more preferably no more than about 0.11%, more preferably no more than about 0.09%, more preferably no more than about 0.08%, more preferably no more than about 0.07%, more preferably no more than about 0.06%, more preferably no more than about 0.05%, more preferably no more than about 0.04%, more preferably no more than about 0.03%, more preferably no more than about 0.02%, and most preferably no more than about 0.01%.

### Pharmacokinetic Embodiments

In some embodiments, after administration of the edaravone liquid formulation of the present invention at doses between about 30 mg and about 300 mg to human subjects, the AUC_{0-∞} (area under the plasma drug concentration-time curve extrapolated to infinity) for edaravone may be observed in the range of about 500 ng·h/mL to about 10,000 ng·h/mL (or from about 0.5 μg·h/mL to about 10 μg·h/mL).

In some embodiments, after oral administration of the edaravone liquid formulation of the present invention at dose from about 80 mg to about 120 mg (e.g., about 80 mg, about 90 mg, about 100 mg, about 105 mg, about 110 mg, about 120 mg) to human subjects, the AUC_{0-∞} for edaravone may be observed in the range of about 1,500 to about 2,500 ng·h/mL.

In some embodiments, after administration of the edaravone liquid formulation of the present invention (e.g., via oral administration or via enteral tube such as nasogastric tube, gastrostomy tube or percutaneous endoscopic gastrostomy tube, jejunostomy tube, or gastrojejunal tube) at a dose of about 80 mg to about 120 mg (e.g., about 80 mg, about 90 mg, about 100 mg, about 105 mg, about 110 mg, about 120 mg, preferably about 105 mg) to human subjects, the AUC_{0-∞} for edaravone may be observed at about 1.0 μg·h/mL, optionally about 1.2 μg·h/mL, optionally about 1.4 μg·h/mL, optionally about 1.6 μg·h/mL, optionally about 1.8 μg·h/mL, optionally about 2.0 μg·h/mL, optionally about 2.2 μg·h/mL, optionally about 2.4 μg·h/mL, optionally about 2.6 μg·h/mL, optionally about 2.8 μg·h/mL, optionally about 3.0 μg·h/mL, optionally about 3.2 μg·h/mL, optionally about 3.4 μg·h/mL, optionally about 3.6 μg·h/mL, optionally about 3.8 μg·h/mL, or optionally about 4.0 μg·h/mL. It is understood that the pharmacokinetic parameters described herein generally correlate with the administered dose (e.g., higher doses generally provide higher AUC values). However, due to significant inter-subject variability in bioavailability, overlapping ranges are contemplated.

In some embodiments, after administration of the edaravone liquid formulation of the present invention at doses between about 30 mg and about 300 mg to human subjects, the Cₘₐₓ (maximum plasma concentration) for edaravone may be observed in the range of about 500 ng/mL to about 10,000 ng/mL (or from about 0.5 μg/mL to about 10 μg/mL).

In some embodiments, after oral administration of the edaravone liquid formulation of the present invention at dose from about 80 mg to about 120 mg (e.g., about 80 mg, about 90 mg, about 100 mg, about 105 mg, about 110 mg, about 120 mg) to human subjects, the Cₘₐₓ for edaravone may be observed in the range of about 1,500 ng/mL to about 2,500 ng/mL.

In some embodiments, after administration of the edaravone liquid formulation of the present invention (e.g., via oral administration or via enteral tube such as nasogastric tube, gastrostomy tube or percutaneous endoscopic gastrostomy tube, jejunostomy tube, or gastrojejunal tube) at a dose of about 80 mg to about 120 mg (e.g., about 80 mg, about 90 mg, about 100 mg, about 105 mg, about 110 mg, about 120 mg, preferably about 105 mg) to human subjects, the Cₘₐₓ for edaravone may be observed at about 1.0 μg/mL to about 4.0 μg/mL, optionally about 1.0 μg/mL, optionally about 1.2 μg/mL, optionally about 1.4 μg/mL, optionally about 1.6 μg/mL, optionally about 1.8 μg/mL, optionally about 2.0 μg/mL, optionally about 2.2 μg/mL, optionally about 2.4 μg/mL, optionally about 2.6 μg/mL, optionally about 2.8 μg/mL, optionally about 3.0 μg/mL, optionally about 3.2 μg/mL, optionally about 3.4 μg/mL, optionally about 3.6 μg/mL, optionally about 3.8 μg/mL, or optionally about 4.0 μg/mL. It is understood that the pharmacokinetic parameters described herein generally correlate with the administered dose (e.g., higher doses generally provide higher Cₘₐₓ values). However, due to significant inter-subject variability in bioavailability, overlapping ranges are contemplated.

In some embodiments, after administration of the edaravone liquid formulation of the present invention at doses between about 30 mg and about 300 mg to human subjects, the Tₘₐₓ (time to reach maximum plasma concentration) for edaravone may be observed in the range of about 0.05 h to about 1.5 h (i.e., about 3 minutes to about 90 minutes).

In some embodiments, after oral administration of the edaravone liquid formulation of the present invention at dose from about 80 mg to about 120 mg (e.g., about 80 mg, about 90 mg, about 100 mg, about 105 mg, about 110 mg, about 120 mg) to human subjects, the Tₘₐₓ for edaravone may be observed in the range of about 0.1 h to about 0.5 h.

In some embodiments of the present invention, it is expected that the administration of the oral liquid formulation at a daily dose selected from the group consisting of about 80 mg, about 90 mg, about 100 mg, about 105 mg, about 110 mg, and about 120 mg of edaravone will result in a mean change from baseline in the Amyotrophic Lateral Sclerosis Functional Rating Scale-Revised (ALSFRS-R) score that is statistically significantly different from placebo (e.g., a slower rate of decline) in a population of subjects with ALS. In particular embodiments, administration of about 105 mg of edaravone is targeted to result in a reduction in the decline of the ALSFRS-R score of at least 33% compared to placebo over a treatment period of 24 weeks or 48 weeks.

In some aspects, the present invention provides an oral liquid dosage form designed to provide a predictable pharmacokinetic profile upon single-dose administration to human subjects. In certain aspects, the invention provides an oral liquid composition that is targeted to be bioequivalent to a reference listed drug product (e.g., RADICAVA ORS® oral suspension or a future oral solid dosage form). The following embodiments further detail the expected pharmacokinetic characteristics of the present invention across a range of dosage strengths.

It is contemplated that a clinical study will be conducted to demonstrate the bioequivalence of an oral liquid pharmaceutical composition of the present invention relative to a reference listed drug product (e.g., an oral suspension containing 105 mg edaravone). The study is proposed as a randomized, open-label, single-dose, two-period, two-treatment crossover study in a cohort of about 24 to 48 healthy adult subjects under fasting conditions. In the first period, subjects are randomly assigned to receive either a single oral dose of the test product (a liquid composition of the present invention containing about 80 mg to about 120 mg of edaravone, preferably about 105 mg) or the reference product of the same strength. Following a washout period of at least 7 days (sufficient to clear the drug given edaravone's half-life), subjects receive alternate treatment in the second period. Serial blood samples for pharmacokinetic analysis are collected at pre-dose (0 hours) and at 0.08, 0.25, 0.5, 0.75, 1, 1.5, 2, 3, 4, 6, 8, 12, 24, and 48-hours post-dose to capture the rapid absorption phase (Tₘₐₓ) and the elimination phase. Plasma concentrations of edaravone are determined using a validated method (e.g. LC-MS/MS method). The pharmacokinetic parameters, including AUC₀₋ₜ, AUC_{0-∞}, and Cₘₐₓ, are calculated for both formulations. It is expected that the 90% confidence intervals for the geometric mean ratios of the test product to the reference product will fall entirely within the 80% to 125% acceptance range for both AUC_{0-∞} and Cₘₐₓ, thereby demonstrating the bioequivalence of the liquid composition.

In addition to a single dose, fasting, crossover study, other scientifically valid designs may be used to establish the bioequivalence of the oral liquid edaravone compositions. These alternatives allow for the assessment of the drug's performance under various conditions. One alternative is a multiple-dose, steady-state crossover study. This design assesses bioequivalence under conditions that reflect drug accumulation following the repeated dosing characteristic of edaravone's therapeutic regimen (e.g., cycles of 14 days on/10 days off). Another alternative is a study conducted under fed conditions. This design evaluates the effect of food on the drug's bioavailability (which is known to significantly reduce edaravone absorption) and can be used to establish bioequivalence when the composition is administered with a meal. Regardless of the specific design employed, the objective is to demonstrate bioequivalence by showing that the 90% confidence interval for the ratio of the geometric means of the key pharmacokinetic parameters (AUC and Cₘₐₓ) falls within the standard acceptance range of about 80% to about 125%.

In one embodiment, the present invention relates to a liquid pharmaceutical composition comprising about 80 mg to about 120 mg of edaravone (e.g., about 105 mg) in a volume ranging from about 1 mL to about 20 mL (e.g., about 5 mL). Following single-dose administration to healthy human subjects, this liquid composition is predicted to demonstrate bioequivalence to an oral reference suspension or tablet, as evidenced by a single-dose geometric mean Area Under the Curve from time zero to infinity (AUC_{0-∞}) and a geometric mean Maximum Plasma Concentration (Cₘₐₓ). Bioequivalence is established when the 90% confidence interval for the ratio of geometric means for both AUC_{0-∞} and Cₘₐₓ falls entirely within the range of about 80% to about 125%.

In some embodiments of the present invention, the invention provides a method for treating Amyotrophic Lateral Sclerosis (ALS). The method comprises administering an oral liquid formulation of edaravone, wherein the administration is expected to result in a slowing of the decline in physical function (as measured by the ALSFRS-R) that is statistically equivalent to the efficacy achieved by administering the reference oral suspension or intravenous formulation at a similar daily dose.

In some embodiments, the present invention relates to methods for treating a condition responsive to free radical scavenging and reduction of oxidative stress, such as ALS, acute ischemic stroke, or Alzheimer's disease, in a human subject in need thereof. The method comprises the intranasal administration of a pharmaceutical composition of edaravone. The following non-limiting prophetic examples describe clinical trial designs intended to demonstrate the efficacy and safety of said intranasal formulation.

In one embodiment, a Phase 3 clinical study could be designed as a multicenter, randomized, double-blind, placebo-controlled, parallel-group trial to evaluate the efficacy and safety of an intranasal edaravone formulation in adult subjects with ALS. The study population comprises subjects with a diagnosis of “Definite” or “Probable” ALS according to the El Escorial revised criteria, a duration of disease of 2 years or less, and a baseline % FVC of at least 80%. Following a screening period and a baseline observation period (e.g., 12 weeks to establish pre-treatment progression), approximately 200 subjects are randomized in a 1:1 ratio to receive either the intranasal edaravone formulation or a matching intranasal placebo. The treatment period has a duration of about 24 to 48 weeks. The primary efficacy endpoint would be the change in the ALSFRS-R score from baseline to the end of the treatment period. Key secondary endpoints would include the time to death or tracheostomy, changes in Percent Predicted Forced Vital Capacity (% FVC), and quality of life measures (e.g., ALSAQ-40).

In another embodiment, the clinical study could be an active-controlled, randomized, double-blind, non-inferiority trial designed to compare the efficacy of the intranasal edaravone formulation to the standard-of-care oral edaravone suspension (105 mg) or oral tablet (90 mg). The objective is to demonstrate that the intranasal formulation is at least as effective as the approved oral formulation. Subjects meeting the inclusion criteria for ALS are randomized to receive either the intranasal edaravone or the oral edaravone suspension with a matching placebo (Double-Dummy Design). The treatment duration is about 24 weeks. The primary endpoint is the slope of decline in the ALSFRS-R score. Non-inferiority will be established if the lower bound of the 95% confidence interval for the difference in ALSFRS-R slope between the intranasal and oral treatment groups is within a pre-specified non-inferiority margin. Secondary endpoints may include assessments of patient preference (e.g., ease of use for dysphagic patients) and speed of absorption (Tₘₐₓ).

In yet another embodiment, the invention provides for a three-arm, placebo- and active-controlled clinical study to simultaneously establish superiority over placebo and characterize the therapeutic profile relative to the oral suspension. Approximately 300 subjects are randomized in a 2:2:1 ratio to one of three arms: (1) intranasal edaravone, (2) oral edaravone suspension (105 mg), or (3) placebo. The study duration is 24 weeks. The primary efficacy endpoint is the change from baseline in the ALSFRS-R score. A hierarchical statistical testing procedure is employed, first testing the superiority of intranasal edaravone to placebo. If this comparison is statistically significant, a subsequent test for the non-inferiority or superiority of the intranasal formulation to the oral suspension is performed.

In another embodiment, the invention provides for a long-term, open-label extension (OLE) study to assess the safety and durability of the treatment effect of the intranasal or oral liquid edaravone formulation. Subjects who have completed any of the preceding controlled Phase 3 studies are eligible to enroll. All subjects in the OLE study receive active edaravone for a duration of about 12 to 24 months (or until disease progression requires withdrawal). The primary endpoints are the incidence, severity, and type of treatment-emergent adverse events, with a particular focus on long-term safety (including renal function monitoring). Secondary endpoints include the sustained effect on ALSFRS-R slope and survival analysis.

In some embodiments of the present invention, therapeutic equivalence could be established by achieving a bioequivalent pharmacokinetic (PK) profile. The oral liquid formulation, when administered at a dose strength selected from the group consisting of about 80 mg, about 90 mg, about 100 mg, about 105 mg, about 110 mg, and about 120 mg of edaravone, is expected to provide a mean area under the plasma concentration-time curve (AUC_{0-∞}) and a mean maximum plasma concentration (Cₘₐₓ) where the 90% confidence intervals for the ratio of the geometric means between the liquid and the reference formulation (e.g., Radicava ORS®) fall within the range of 80% to 125%.

In certain embodiments, the invention provides a method for treating ALS or reducing neuronal oxidative stress in a subject. The method comprises administering an intranasal formulation of edaravone, wherein said administration is expected to result in a superior clinical outcome compared to the administration of a standard oral edaravone formulation (e.g., 105 mg oral suspension). This superiority is expected even when the total daily intranasal dose is substantially similar to or lower than the subject's total daily oral dose.

In a specific prophetic example, the superior clinical outcome would be defined by a statistically equivalent or superior preservation of motor neurons (as measured by ALSFRS-R or biomarkers such as neurofilament light chain), coupled with lower systemic plasma concentrations and reduced renal burden. For example, it is hypothesized that subjects administered the intranasal formulation at a lower daily dose (e.g., about 4 mg to about 20 mg/day) would achieve CNS oxidative stress reduction comparable to the 105 mg oral dose. However, pharmacokinetic analysis is expected to confirm that this effect would be achieved with a mean systemic AUC approximately 50-90% lower than that of subjects administered the 105 mg oral dose. This superior therapeutic profile would minimize the concentration of edaravone and its metabolites passing through the kidneys, thereby reducing the potential for renal toxicity suitable for patients with renal impairment. The intranasal administration thereby provides a more targeted treatment for ALS, enhancing the safety margin for long-term chronic therapy. Similar plausible embodiments may be applicable to other neurodegenerative diseases treatable by edaravone such as acute Ischemic Stroke (AIS), Alzheimer's Disease (AD), Autism Spectrum Disorder (ASD), Acute Mountain Sickness (AMS), Hearing Loss and Vestibular Disorders, Contrast-Induced Acute Kidney Injury (CI-AKI), Parkinson's Disease (PD), and Traumatic Brain Injury (TBI).

In certain embodiments, the invention provides a method for treating ALS in a subject, particularly a subject with renal impairment or at risk of renal toxicity. The method comprises administering a daily dose of a liquid formulation of edaravone via an intranasal route, wherein said administration is expected to result in a superior clinical outcome characterized by an improved safety profile. This superiority is targeted to be achieved while simultaneously reducing total systemic drug exposure when compared to a therapeutically effective oral suspension (105 mg).

In a specific prophetic example, the superior clinical outcome would be defined by a statistically equivalent or superior preservation of motor neurons (as measured by ALSFRS-R or biomarkers such as neurofilament light chain), coupled with lower systemic plasma concentrations and reduced renal burden. For example, it is hypothesized that subjects administered the intranasal formulation at a lower daily dose (e.g., about 4 mg to about 20 mg/day) would achieve CNS oxidative stress reduction comparable to the 105 mg oral dose. However, pharmacokinetic analysis is expected to confirm that this effect would be achieved with a mean systemic AUC approximately 50-90% lower than that of subjects administered the 105 mg oral dose. This superior therapeutic profile would minimize the concentration of edaravone and its metabolites passing through the kidneys, thereby reducing the potential for renal toxicity suitable for patients with renal impairment. The intranasal administration thereby provides a more targeted treatment for ALS, enhancing the safety margin for long-term chronic therapy. Similar plausible embodiments may be applicable to other diseases and conditions treatable by edaravone, including Acute Ischemic Stroke (AIS), Alzheimer's Disease (AD), Autism Spectrum Disorder (ASD), Acute Mountain Sickness (AMS), Hearing Loss and Vestibular Disorders, Contrast-Induced Acute Kidney Injury (CI-AKI), Parkinson's Disease (PD), and Traumatic Brain Injury (TBI).

It is understood that the clinical study designs described herein (including patient sample sizes, inclusion/exclusion criteria, specific endpoints, and titration schedules) are exemplary and may be modified based on guidance from regulatory agencies (e.g., FDA, EMA, PMDA) or statistical power calculations during the course of development. Such modifications to the trial design do not alter the underlying method of treatment or the therapeutic scope of the pharmaceutical compositions described herein. For example, while a sample size of 200 subjects is proposed, the actual study may comprise fewer (e.g., 50-100) or more (e.g., 300-500) subjects as required to demonstrate statistical significance. Similarly, while ALSFRS-R is a preferred endpoint, other validated clinical endpoints (e.g., Combined Assessment of Function and Survival (CAFS), Slow Vital Capacity (SVC)) may be utilized to demonstrate efficacy.

### Method of Use Embodiments

In some embodiments of the present invention, an oral liquid pharmaceutical composition is provided, comprising: (a) edaravone at a concentration sufficient to deliver a unit dose selected from a group consisting of about 80 mg, about 90 mg, about 100 mg, about 105 mg, about 110 mg, and about 120 mg; (b) at least one pharmaceutically acceptable liquid carrier; and (c) one or more excipients, wherein the dose volume is from about 1 mL to about 20 mL. This composition is formulated to provide a pharmacokinetic and therapeutic profile equivalent to that of a reference oral suspension or intravenous formulation at a similar daily dose, while minimizing the administration volume to accommodate patients with dysphagia.

For the purposes of the present invention, the term “manageable volume” as used herein is defined as a quantity of the oral liquid pharmaceutical composition (e.g., less than about 20 mL, preferably less than about 10 mL) that is suitable for convenient, safe, and accurate administration to a subject, particularly for self-administration or administration by a caregiver to a patient with bulbar onset ALS or severe dysphagia. The volume is selected to minimize aspiration risk and promote patient compliance.

In an embodiment, the edaravone liquid formulation of the present invention is administered at a therapeutically effective dose to patients in need thereof.

In some embodiments, the edaravone liquid formulation of the present invention is administered to the patient in an amount from about 30 mg to about 300 mg per dose, such as about 60 mg, about 80 mg, about 90 mg, about 100 mg, about 105 mg, about 110 mg, about 120 mg, about 140 mg, or about 160 mg per dose.

In an embodiment, the present invention provides a method of treating a disease or condition selected from the group consisting of Amyotrophic Lateral Sclerosis (ALS), Acute Ischemic Stroke (AIS), Alzheimer's Disease (AD), Autism Spectrum Disorder (ASD), Acute Mountain Sickness (AMS), Hearing Loss and Vestibular Disorders, Contrast-Induced Acute Kidney Injury (CI-AKI), Parkinson's Disease (PD), and Traumatic Brain Injury (TBI), comprising administering a therapeutically effective amount of the edaravone liquid formulation to a patient in need thereof.

In one embodiment, the formulation is administered orally or via an enteral feeding tube (e.g., nasogastric, PEG, or jejunostomy tube), in a volume of about 1 mL to about 20 mL delivering about 80 mg to about 120 mg edaravone per dose.

In another embodiment, the formulation is administered intranasally, wherein each actuation delivers about 0.05 mL to about 0.15 mL of formulation, corresponding to about 1 mg to about 20 mg of edaravone. The total therapeutic dose (e.g., 4 mg to 40 mg) may be administered via single or multiple sprays per nostril.

In yet another embodiment, the formulation is administered via parenteral injection (e.g., intravenous, intramuscular, or subcutaneous), formulated as a sterile solution or depot injection.

In some embodiments of the present invention, the edaravone liquid formulation is administered to the patient at a dosing frequency of once a day, optionally twice a day, optionally in treatment cycles (e.g., 14 days of daily administration followed by 10 or 14 days of drug-free period), or continuously, depending on the indication (e.g., continuous daily dosing for AD or PD; cyclic dosing for ALS).

In some embodiments, the present invention provides the method of use of the edaravone liquid formulation comprising steps of: (i) opening the child-resistant cap of the bottle; (ii) drawing the required dose (e.g., 5 mL containing 105 mg edaravone) into a graduated oral syringe; (iii) dispensing the drawn liquid formulation into the oral cavity; and (iv) optionally drinking water to rinse the oral cavity.

In an embodiment of the present invention, the liquid formulation of edaravone is administered to patients using syringe and delivery tube, such as nasogastric tube, gastrostomy tube, jejunostomy tube, or gastrojejunal tube.

In yet other embodiments, the present invention provides the method of use of the edaravone liquid formulation comprising steps of: (i) opening the child-resistant cap of the bottle; (ii) drawing the required dose into a graduated syringe; (iii) connecting the syringe with a delivery tube (such as a nasogastric tube, gastrostomy tube (PEG), or jejunostomy tube); (iv) pressing the piston of the syringe to deliver the liquid formulation; (v) optionally flushing the delivery tube with water.

In some embodiments, the present invention relates to the method of use of the edaravone liquid formulation comprising steps of: (i) placing the actuator in the nasal cavity or nostril; and (ii) actuating the device to deliver the spray of liquid formulation into the nasal cavity or on the nasal mucosa. This method is particularly suitable for patients with dysphagia or those requiring rapid central nervous system delivery.

In some embodiments, the present invention provides a method of treating a subject in need thereof (e.g., for ALS), comprising administering a stable liquid formulation of edaravone of the present invention suitable for oral or intragastrical administration, wherein the administration is timed relative to a first time interval from a consumption of a meal by the subject to the administration of the liquid pharmaceutical composition. In one embodiment, the first time interval is determined based on the caloric and fat content of the meal: (i) wherein for a high-fat meal (e.g., in a range of about 800 to 1000 calories with approximately 50% fat), the first time interval is about 8 hours before the administration of the liquid pharmaceutical composition; (ii) wherein for a low-fat meal (e.g., in a range of about 400 to 500 calories with approximately 25% fat), the first time interval is about 4 hours before the administration; or (iii) wherein for a caloric supplement (e.g., approximately 250 calories), the first time interval is about 2 hours before the administration. Administration according to this schedule ensures that a decrease in Cₘₐₓ is less than 20% with respect to Cₘₐₓ in a same time range in fasting for 10 hours or longer, and/or that a decrease in AUC is less than 10% with respect to AUC in a same time range in fasting for 10 hours or longer.

In some other embodiments, the present invention provides a method of treating a subject in need thereof (e.g., for ALS), comprising administering a stable liquid formulation of edaravone of the present invention suitable for oral or intragastrical administration, wherein the administration is timed relative to a first time interval from a consumption of a meal by the subject to the administration of the liquid pharmaceutical composition. In one embodiment, the first time interval is determined based on the caloric and fat content of the meal: (i) the first time interval for the consumption of a high-fat meal (e.g., in a range of about 800 to 1000 calories with 50% fat) is less than about 8 hours (such as about 2 hours, optionally about 4 hours, optionally about 5 hours, optionally about 6 hours, optionally about 7 hours) before the administration of the liquid pharmaceutical composition; (ii) the first time interval for the consumption of a low-fat meal (e.g., in a range of about 400 to 500 calories with 25% fat) is less than about 4 hours (such as about 1 hour, optionally about 2 hours, optionally about 3 hours) before the administration; or (iii) the first time interval for the consumption of a caloric supplement (e.g., of about 250 calories) is less than about 2 hours (such as about 1 hour, optionally between about 1 hour to about 2 hours) before the administration. In such embodiments, the administration maintains a pharmacokinetic profile wherein a decrease in Cmax is less than 20% with respect to Cmax in a same time range in fasting for 10 hours or longer, and/or wherein a decrease in AUC is less than 10% with respect to AUC in a same time range in fasting for 10 hours or longer.

In some embodiments, the edaravone liquid formulation of the present invention may be administered to patients across various age groups, including adult and geriatric populations. Dosing and frequency may be adjusted according to the age, weight, and clinical condition of the patient (e.g., renal function) to ensure safety and efficacy.

In further embodiments, the edaravone liquid formulation of the present invention may be co-administered with other medications as part of a combination therapy regimen (e.g., with Riluzole, Sodium Phenylbutyrate/Taurursodiol, or antioxidant supplements), provided such combinations are compatible and do not adversely affect the pharmacological activity of edaravone.

In some embodiments of the present invention, the liquid formulation of edaravone is administered to the patient in need thereof via the oral route of administration (as an oral liquid solution) in a volume ranging from about 1 mL to about 20 mL per dose. Table 1 illustrates oral liquid formulations of edaravone with different concentrations and specific volumes of dose to be delivered, respectively.

TABLE 1
Examples for use of present invention via oral administration

| Volume to | | Edaravone Concentration in mg/mL (Single Oral Liquid Dose) | | | | | | | |
|---|---|---|---|---|---|---|---|---|---|
| be delivered | | 30 mg | 60 mg | 80 mg | 90 mg | 100 mg | 105 mg | 120 mg | 140 mg |
| per dose | | dose | dose | dose | dose | dose | dose | dose | dose |
| 1.0 | mL | 30 | 60 | 80 | 90 | 100 | 105 | 120 | 140 |
| 2.5 | mL | 12 | 24 | 32 | 36 | 40 | 42 | 48 | 56 |
| 5.0 | mL | 6 | 12 | 16 | 18 | 20 | 21 | 24 | 28 |
| 10.0 | mL | 3 | 6 | 8 | 9 | 10 | 10.5 | 12 | 14 |
| 15.0 | mL | 2 | 4 | 5.33 | 6 | 6.67 | 7 | 8 | 9.33 |
| 20.0 | mL | 1.5 | 3 | 4 | 4.5 | 5 | 5.25 | 6 | 7 |

The following tables illustrate nasal spray formulations of edaravone with different concentrations and specific volumes of dose to be delivered, respectively.

TABLE 2
Examples for use of present invention via
intranasal administration (Single Spray)

| | Edaravone Concentration in | | | | | |
|---|---|---|---|---|---|---|
| Volume to | mg/mL (Single Nasal Spray per Dose) | | | | | |
| be delivered | 1 mg | 2 mg | 4 mg | 5 mg | 10 mg | 20 mg |
| per Spray | dose | dose | dose | dose | dose | dose |
| 0.050 mL | 20 | 40 | 80 | 100 | 200 | 400 |
| 0.100 mL | 10 | 20 | 40 | 50 | 100 | 200 |
| 0.125 mL | 8 | 16 | 32 | 40 | 80 | 160 |
| 0.150 mL | 6.7 | 13.3 | 26.7 | 33.3 | 66.7 | 133.3 |

TABLE 3
Examples for use of present invention via
intranasal administration (Two Spray)

| | Edaravone Concentration in | | | | | |
|---|---|---|---|---|---|---|
| Volume to | mg/mL (Two Nasal Spray per Dose) | | | | | |
| be delivered | 1 mg | 2 mg | 4 mg | 5 mg | 10 mg | 20 mg |
| per Spray | dose | dose | dose | dose | dose | dose |
| 0.050 mL | 10 | 20 | 40 | 50 | 100 | 200 |
| 0.100 mL | 5 | 10 | 20 | 25 | 50 | 100 |
| 0.125 mL | 4 | 8 | 16 | 20 | 40 | 80 |
| 0.150 mL | 3.33 | 6.67 | 13.33 | 16.67 | 33.33 | 66.7 |

The following tables illustrate various doses and concentrations of edaravone provided as a liquid formulation filled in hard capsules (Table 4) or soft gelatin capsules (Table 5). It is understood that the fill volumes provided are exemplary and may vary based on the specific density of the liquid vehicle used.

TABLE 4
Examples for use of present invention via liquid-filled hard capsule

| | Fill | Edaravone approx. concentration in mg/mL (One capsule per dose) | | | | | |
|---|---|---|---|---|---|---|---|
| Capsule | Volume | 30 mg | 60 mg | 90 mg | 100 mg | 105 mg | 120 mg |
| Size | (mL) | dose | dose | dose | dose | dose | dose |
| Size 2 | ~0.37 mL | 81.1 | 162.2 | 243.2 | 270.3 | 283.8 | 324.3 |
| Size 1 | ~0.50 mL | 60 | 120 | 180 | 200 | 210 | 240 |
| Size 0 | ~0.68 mL | 44.1 | 88.2 | 132.4 | 147.1 | 154.4 | 176.5 |
| Size 00 | ~0.95 mL | 31.6 | 63.2 | 94.7 | 105.3 | 110.5 | 126.3 |

TABLE 5
Examples for use of present invention via liquid-filled soft gelatin capsule

| | Approx. Fill | Edaravone approx. concentration in mg/mL (One capsule per dose) | | | | | |
|---|---|---|---|---|---|---|---|
| Softgel Size | Volume | 30 mg | 60 mg | 90 mg | 100 mg | 105 mg | 120 mg |
| & Shape | (mL) | dose | dose | dose | dose | dose | dose |
| 5 Minim Oval | ~0.30 mL | 100.0 | 200.0 | 300.0 | 333.3 | 350.0 | 400.0 |
| 10 Minim Oval | ~0.61 mL | 49.2 | 98.4 | 147.5 | 163.9 | 172.1 | 196.7 |
| 12 Minim Oblong | ~0.74 mL | 40.5 | 81.1 | 121.6 | 135.1 | 141.9 | 162.2 |
| 20 Minim Oblong | ~1.23 mL | 24.4 | 48.8 | 73.2 | 81.3 | 85.4 | 97.6 |

In some embodiments, the preferred formulation for delayed-release oral delivery is a liquid-filled capsule comprising solubilized and/or suspended edaravone in a non-aqueous vehicle (e.g., polyethylene glycol or triglycerides). This dosage form is designed to bypass the stomach and release the edaravone in the upper intestine, potentially enhancing bioavailability and reducing gastric irritation.

In yet another embodiment, the invention is an in-situ gelling liquid formulation for intranasal administration. The formulation is a low-viscosity liquid at storage temperature (e.g., 2-8° C. or about 15-25° C.) for ease of atomization from a spray device but rapidly transforms into a high-viscosity gel upon contact with the nasal mucosa at body temperature. This is achieved using a thermo-responsive polymer, such as a poloxamer (e.g., Poloxamer 407), or an ion-sensitive polymer, such as gellan gum. This embodiment provides the benefits of prolonged residence time on the nasal mucosa, enhancing absorption and potential nose-to-brain delivery of edaravone.

In another embodiment, the invention provides a method for treating ALS in a human subject. The method comprises administering to said subject the viscous intranasal edaravone formulation described herein. The administration of said intranasal formulation results in a therapeutic effect equivalent to or superior to an oral dose, with potentially reduced systemic exposure and lower risk of renal toxicity.

### Working Examples

The present invention is further illustrated by, but is by no means limited to, the following examples. It will be appreciated that where typical or preferred process conditions are given, other process conditions can also be used unless otherwise stated. Materials used in the examples set forth below were provided by commercial suppliers. It is preferred that ingredients, used in edaravone liquid formulations, comply with the compendial monograph such as USP, Ph. Eur., and/or JP, and ICH guidelines for residual process related impurities (elemental impurities and residual solvents), if available.

Purity and Impurity analysis reported in the examples were performed using a reverse-phase high-performance chromatography (HPLC) system with UV detection. The method utilized a C18 column with 5 μm particles and 250×4.6 mm dimensions, detection at about 240 nm wavelength, and using gradient elution of an aqueous buffer at pH about 5.8 and an organic solvent (acetonitrile and methanol).

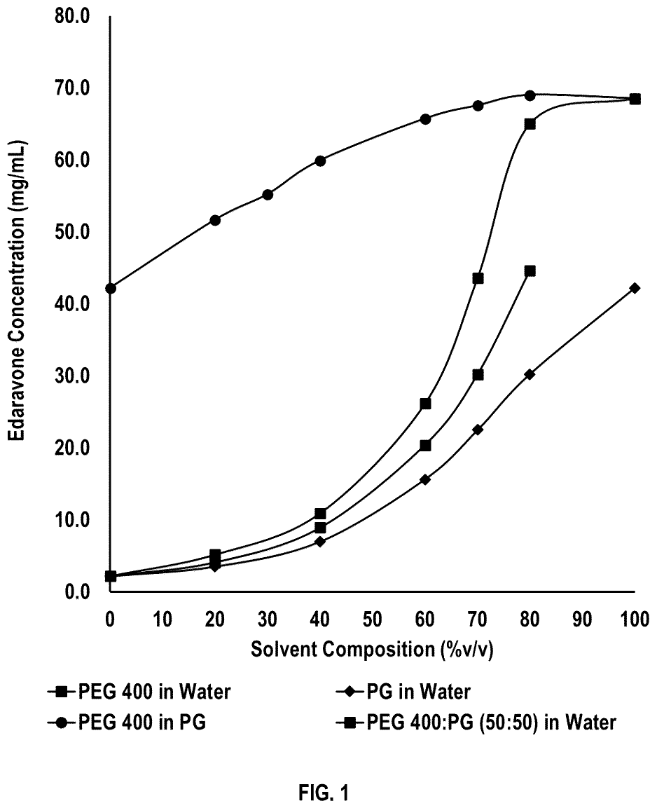

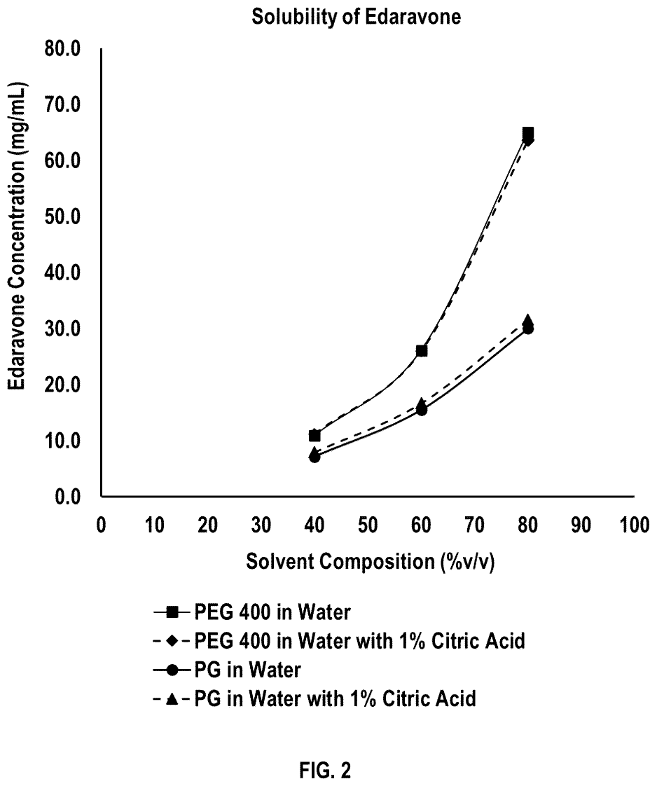

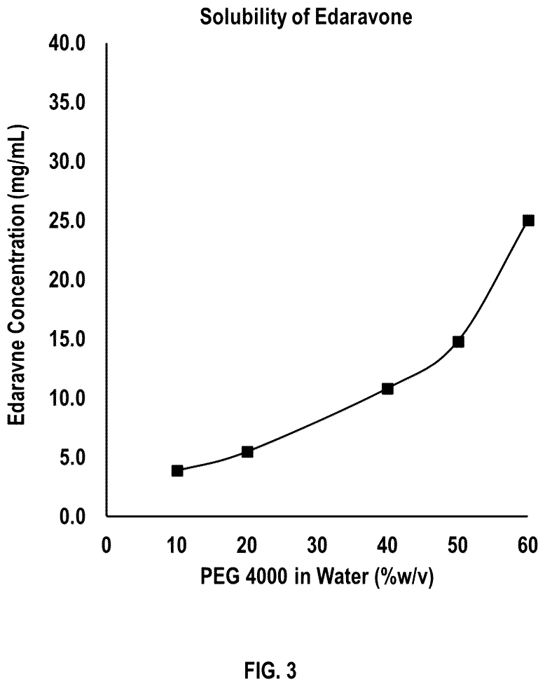

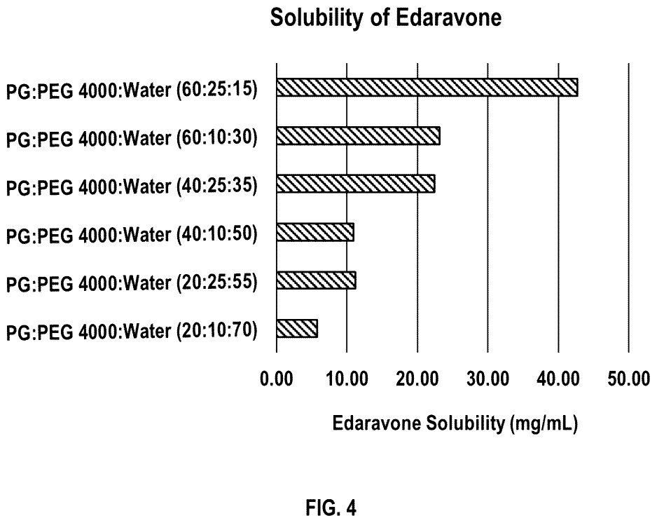

Recent prior art (Parikh et al. 2017) reported significantly low solubility of Edaravone in propylene glycol (5.93 mg/mL) and PEG 400 (6.40 mg/mL). These reported values are drastically lower than the solubility in ethanol (e.g., ~70 mg/mL from Parikh et al., ~52 mg/g from Dube et al., and ~111 mg/mL from Yamada et al). Consequently, the prior art establishes a technical prejudice suggesting that propylene glycol and PEG 400 are unsuitable as primary solvents for high-concentration formulations. Based on these teachings, the ability to achieve high concentrations of edaravone in these vehicles without significant ethanol was unpredictable. Contrary to these reports, the Inventor's solubility studies (Table 6 and FIG. 1-4) surprisingly demonstrated that edaravone has a solubility of about 42.2 mg/mL in propylene glycol and about 68.5 mg/mL in PEG 400-values significantly higher than previously disclosed. Furthermore, as presented in FIG. 1, solubility increases non-linearly when the organic solvent exceeds about 400% v/v in water.

TABLE 6
Equilibrium solubility of edaravone in various solvent systems.

| | |
|---|---|
| | Equilibrium |
| | Solubility of |
| Solvent System | Edaravone (mg/mL) |

| | | |
|---|---|---|
| PEG 400 (100%) | ~68.5 | mg/mL |
| PEG 400:Water (80:20) (% v/v) | ~65.0 | mg/mL |
| PEG 400:Water (70:30) (% v/v) | ~43.6 | mg/mL |
| PEG 400:Water (60:40) (% v/v) | ~26.1 | mg/mL |
| PEG 400:Water (40:60) (% v/v) | ~10.9 | mg/mL |
| PEG 400:Water (20:80) (% v/v) | ~5.2 | mg/mL |
| PG (100%) | ~42.2 | mg/mL |
| PG:Water (80:20) (% v/v) | ~30.2 | mg/mL |
| PG:Water (70:30) (% v/v) | ~22.5 | mg/mL |
| PG:Water (60:40) (% v/v) | ~15.6 | mg/mL |
| PG:Water (40:60) (% v/v) | ~7.0 | mg/mL |
| PG:Water (20:80) (% v/v) | ~3.5 | mg/mL |
| PEG 400:PG (80:20) | ~69.0 | mg/mL |
| PEG 400:PG (70:30) | ~67.5 | mg/mL |
| PEG 400:PG (60:40) | ~65.7 | mg/mL |
| PEG 400:PG (40:60) | ~59.9 | mg/mL |
| PEG 400:PG (30:70) | ~55.2 | mg/mL |
| PEG 400:PG (20:80) | ~51.7 | mg/mL |
| PEG 400:PG:Water (40:40:20) (% v/v/v) | ~44.6 | mg/mL |
| PEG 400:PG:Water (35:35:30) (% v/v/v) | ~30.2 | mg/mL |
| PEG 400:PG:Water (30:30:40) (% v/v/v) | ~20.4 | mg/mL |
| PEG 400:PG:Water (20:20:60) (% v/v/v) | ~8.9 | mg/mL |
| PEG 400:PG:Water (10:10:80) (% v/v/v) | ~4.1 | mg/mL |
| Water | ~2.2 | mg/mL |
| PG:PEG4000:Water (20:10:70) (% v/w/v) | ~5.8 | mg/mL |
| PG:PEG4000:Water (20:25:55) (% v/w/v) | ~11.2 | mg/mL |
| PG:PEG4000:Water (40:10:50) (% v/w/v) | ~10.9 | mg/mL |
| PG:PEG4000:Water (40:25:35) (% v/w/v) | ~22.4 | mg/mL |
| PG:PEG4000:Water (60:10:30) (% v/w/v) | ~23.2 | mg/mL |
| PG:PEG4000:Water (60:25:15) (% v/w/v) | ~42.7 | mg/mL |

To evaluate the stability of edaravone in propylene glycol and polyethylene glycol at high concentration, the inventor performed heat treatment study suggested by Yamada et al. in Japanese Patent no. JP4850426 for ethanol-based formulation. In this study the inventor didn't use any antioxidant, stabilizer or water to understand the interaction of edaravone with the solvents. Example 1 (A-E) were prepared by dissolving required amount of edaravone in vehicles listed in Table 7 to achieve 21 mg/mL edaravone concentration. The solution was filled in 2R USP Type I clear glass vial with 2 mL fill volume, stoppered without nitrogen overlay, and sealed with aluminum crimp seal. The vials were heated at 121° C. for 20 minutes using autoclave in upright position. Test results for example 1 are provided in Table 8.

TABLE 7
Compositions for edaravone liquid
formulations of example 1 (A-E).

| Example | Edaravone | Vehicle |
|---|---|---|
| No. | Qty | (q.s. to 1 mL) |
| 1A | 21 mg | Ethanol |
| 1B | 21 mg | Propylene glycol |
| 1C | 21 mg | Polyethylene glycol 200 |
| 1D | 21 mg | Polyethylene glycol 300 |
| 1E | 21 mg | Polyethylene glycol 400 |

TABLE 8
Test results for example 1 (A-E) after
heat treatment at 121° C./20 min.

| Example | | % Total |
|---|---|---|
| No. | Visual Description | Impurities |
| 1A | Clear, colorless solution | 0.5 |
| 1B | Clear, very light green | 1.3 |
| | color solution | |
| 1C | Clear, yellow color solution | 5.8 |
| 1D | Clear, yellow color solution | 5.0 |
| 1E | Clear, orange color solution | 2.3 |

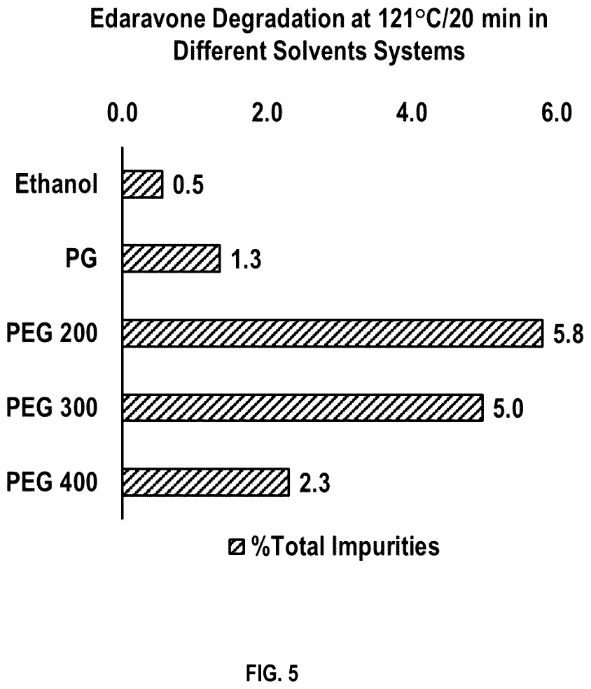

The edaravone was found significantly stable in ethanol (remained clear, colorless) which is consistent with teachings from Yamada et al. and Dube et al. in ethanol-based formulations. However, the drug showed significant degradation in propylene glycol and polyethylene glycol (significant discoloration) compared to ethanol (FIG. 5). This distinct degradation behavior indicates that the stabilization strategies successful in ethanol-based formulations cannot be extrapolated to propylene glycol and polyethylene glycol vehicles with predictable results.

Example 2 (A-G) were prepared to evaluate the effect of solvent ratio on stability of edaravone by using heat treatment suggested by Yamada et al. Required amount of edaravone was dissolved in organic solvents, volume was adjusted using water or other organic solvent to achieve specified ratio of solvents given in Table 9, and 15 mg/mL edaravone concentration in final vehicles. Clear, colorless solutions were obtained for all compositions. The solution was filled in 2R USP Type I clear glass vial with 1 mL fill volume, stoppered without nitrogen overlay (ambient condition), and sealed with aluminum crimp seal. The vials were heated at 121° C. for 20 minutes by autoclave in upright position. Test results for example 2, are provided in Table 10.

TABLE 9
Compositions for edaravone liquid
formulations of Example 2 (A-G).

| Example | | |
|---|---|---|
| No. | Edaravone | Vehicle (ratio in % v/v) |
| 2A1 | 15 mg/mL | Ethanol:Water (50:50) |
| 2A2 | 15 mg/mL | Ethanol:Water (60:40) |
| 2A3 | 15 mg/mL | Ethanol:Water (80:20) |
| 2A4 | 15 mg/mL | Ethanol:Water (100:00) |
| 2B1 | 15 mg/mL | Propylene Glycol:Water (60:40) |
| 2B2 | 15 mg/mL | Propylene Glycol:Water (80:20) |
| 2B3 | 15 mg/mL | Propylene Glycol:Water (100:00) |
| 2C1 | 15 mg/mL | PEG 400:Water (60:40) |
| 2C2 | 15 mg/mL | PEG 400:Water (80:20) |
| 2C3 | 15 mg/mL | PEG 400:Water (100:00) |
| 2D1 | 15 mg/mL | Propylene Glycol:PEG 400:Water |
| | | (20:60:20) |
| 2D2 | 15 mg/mL | Propylene Glycol:PEG 400:Water |
| | | (40:40:20) |
| 2D3 | 15 mg/mL | Propylene Glycol:PEG 400:Water |
| | | (60:20:20) |
| 2E1 | 15 mg/mL | Ethanol:Propylene Glycol (10:90) |
| 2E2 | 15 mg/mL | Ethanol:PEG 400 (10:90) |
| 2E3 | 15 mg/mL | Ethanol:Propylene Glycol:PEG 400 |
| | | (10:45:45) |
| 2F1 | 15 mg/mL | Ethanol:Propylene Glycol:Water |
| | | (10:70:20) |
| 2F2 | 15 mg/mL | Ethanol:PEG 400:Water (10:70:20) |
| 2F3 | 15 mg/mL | Ethanol:Propylene Glycol:PEG 400:Water |
| | | (10:35:35:20) |
| 2G1 | 15 mg/mL | Ethanol:Propylene Glycol (50:50) |
| 2G2 | 15 mg/mL | Ethanol:PEG 400 (50:50) |

TABLE 10
Test results for Example 2 (A-G) after
heat treatment at 121° C./20 min.

| | | | % Highest | |
|---|---|---|---|---|
| Example | | | Single | % Total |
| No. | Description | % Purity | Impurity | Impurities |
| 2A1 | Clear, colorless solution | 99.7 | 0.11 | 0.3 |
| 2A2 | Clear, colorless solution | 99.7 | 0.11 | 0.3 |
| 2A3 | Clear, colorless solution | 99.6 | 0.15 | 0.4 |
| 2A4 | Clear, colorless solution | 99.5 | 0.21 | 0.5 |
| 2B1 | Clear, colorless solution | 99.5 | 0.17 | 0.5 |
| 2B2 | Clear, very light green | 99.0 | 0.33 | 1.0 |
| | color solution | | | |
| 2B3 | Clear, light green color | 98.1 | 0.90 | 1.9 |
| | solution | | | |
| 2C1 | Clear, yellow color | 98.6 | 0.67 | 1.4 |
| | solution | | | |
| 2C2 | Clear, orange color | 97.2 | 1.42 | 2.8 |
| | solution | | | |
| 2C3 | Clear, orange color | 96.1 | 2.14 | 3.9 |
| | solution | | | |
| 2D1 | Clear, light brown | 97.4 | 1.26 | 2.6 |
| | color solution | | | |
| 2D2 | Clear, green color | 97.9 | 0.95 | 2.1 |
| | solution | | | |
| 2D3 | Clear, green color | 98.2 | 0.74 | 1.8 |
| | solution | | | |
| 2E1 | Clear, very light- | 98.2 | 0.88 | 1.8 |
| | yellow color solution | | | |
| 2E2 | Clear, orange color | 96.6 | 1.83 | 3.4 |
| | solution | | | |
| 2E3 | Clear, brown color | 97.9 | 0.97 | 2.1 |
| | solution | | | |
| 2F1 | Clear, colorless solution | 99.1 | 0.34 | 0.9 |
| 2F2 | Clear, yellow color | 97.8 | 1.09 | 2.2 |
| | solution | | | |
| 2F3 | Clear, green color | 98.3 | 0.75 | 1.7 |
| | solution | | | |
| 2G1 | Clear, very light- | 97.7 | 1.20 | 2.3 |
| | yellow color solution | | | |
| 2G2 | Clear, yellow color | 98.1 | 1.03 | 1.9 |
| | solution | | | |

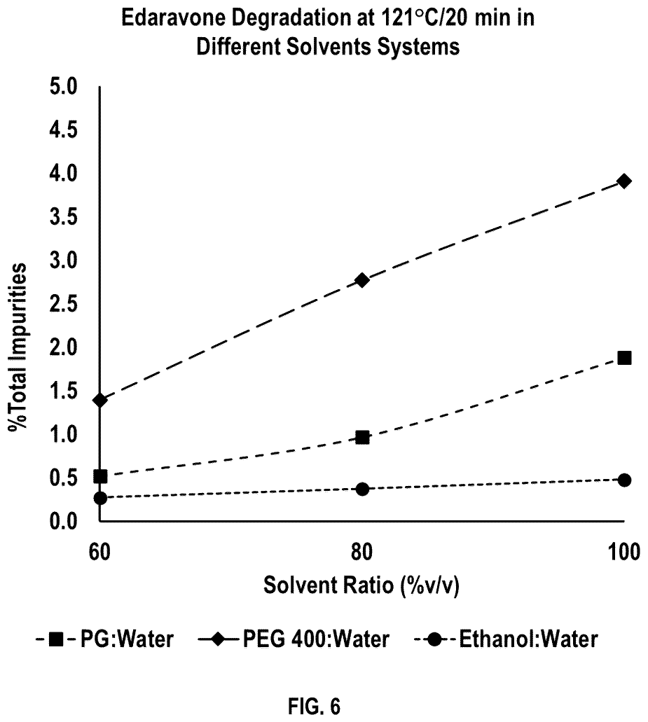

Test results (2A-2D), for example 2 (FIG. 6) suggest that edaravone degradation increases as the organic solvent concentration increases in water and this rate is significantly higher in polyethylene glycol (highest) and propylene glycol compared to ethanol.

Example 3 describes edaravone liquid compositions prepared using ingredients listed in the table 11 to evaluate the role of antioxidant. Required quantity of edaravone was dissolved in required volume of organic solvents. In separate vessel, where applicable, required quantity of sodium metabisulfite was dissolved in water. Aqueous solution was then added to edaravone solution and mixed to obtain homogenous solution. The solution was filled in 2R USP Type I clear glass vial with 1 mL fill volume, stoppered without nitrogen overlay, and sealed with aluminum crimp seal. The vials were heated at 121° C. for 20 minutes by autoclave. Test results for example 3, are provided in Table 12.

TABLE 11
Compositions for edaravone liquid
formulations of Example 3 (A-E).

| Example | | Vehicle | |
|---|---|---|---|
| No. | Edaravone | (q.s. to 1 mL) | Excipient |
| 3A1 | 21 mg/mL | Ethanol:Water | — |
| | | (70:30) (% v/v) | |
| 3A2 | 21 mg/mL | Ethanol:Water | 0.1 mg/mL sodium |
| | | (70:30) (% v/v) | metabisulfite |
| 3A3 | 21 mg/mL | Ethanol:Water | 1 mg/mL sodium |
| | | (70:30) (% v/v) | metabisulfite |
| 3A4 | 21 mg/mL | Ethanol:Water | 5 mg/mL sodium |
| | | (70:30) (% v/v) | metabisulfite |
| 3A5 | 21 mg/mL | Ethanol:Water | 10 mg/mL sodium |
| | | (70:30) (% v/v) | metabisulfite |
| 3B1 | 21 mg/mL | PG:Water | — |
| | | (70:30) (% v/v) | |
| 3B2 | 21 mg/mL | PG:Water | 0.1 mg/mL sodium |
| | | (70:30) (% v/v) | metabisulfite |
| 3B3 | 21 mg/mL | PG:Water | 1 mg/mL sodium |
| | | (70:30) (% v/v) | metabisulfite |
| 3B4 | 21 mg/mL | PG:Water | 5 mg/mL sodium |
| | | (70:30) (% v/v) | metabisulfite |
| 3B5 | 21 mg/mL | PG:Water | 10 mg/mL sodium |
| | | (70:30) (% v/v) | metabisulfite |
| 3C1 | 21 mg/mL | PEG 400:Water | — |
| | | (70:30) (% v/v) | |
| 3C2 | 21 mg/mL | PEG 400:Water | 0.1 mg/mL sodium |
| | | (70:30) (% v/v) | metabisulfite |
| 3C3 | 21 mg/mL | PEG 400:Water | 1 mg/mL sodium |
| | | (70:30) (% v/v) | metabisulfite |
| 3C4 | 21 mg/mL | PEG 400:Water | 5 mg/mL sodium |
| | | (70:30) (% v/v) | metabisulfite |
| 3C5 | 21 mg/mL | PEG 400:Water | 10 mg/mL sodium |
| | | (70:30) (% v/v) | metabisulfite |
| 3D1 | 21 mg/mL | PEG 300:Water | — |
| | | (70:30) (% v/v) | |
| 3D2 | 21 mg/mL | PEG 300:Water | 0.1 mg/mL sodium |
| | | (70:30) (% v/v) | metabisulfite |
| 3D3 | 21 mg/mL | PEG 300:Water | 1 mg/mL sodium |
| | | (70:30) (% v/v) | metabisulfite |
| 3D4 | 21 mg/mL | PEG 300:Water | 5 mg/mL sodium |
| | | (70:30) (% v/v) | metabisulfite |
| 3D5 | 21 mg/mL | PEG 300:Water | 10 mg/mL sodium |
| | | (70:30) (% v/v) | metabisulfite |
| 3E1 | 21 mg/mL | PEG 200:Water | — |
| | | (70:30) (% v/v) | |
| 3E2 | 21 mg/mL | PEG 200:Water | 0.1 mg/mL sodium |
| | | (70:30) (% v/v) | metabisulfite |
| 3E3 | 21 mg/mL | PEG 200:Water | 1 mg/mL sodium |
| | | (70:30) (% v/v) | metabisulfite |
| 3E4 | 21 mg/mL | PEG 200:Water | 5 mg/mL sodium |
| | | (70:30) (% v/v) | metabisulfite |
| 3E5 | 21 mg/mL | PEG 200:Water | 10 mg/mL sodium |
| | | (70:30) (% v/v) | metabisulfite |

TABLE 12
Test results for Example 3 (A-E) after heat treatment at 121° C./20 min.

| Example | | | % Highest | % Total |
|---|---|---|---|---|
| No. | Description | % Purity | Single Impurity | Impurities |
| 3A1 | Clear, colorless solution | 99.7 | 0.12 | 0.3 |
| 3A2 | Clear, colorless solution | 99.8 | 0.11 | 0.2 |
| 3A3 | Clear, colorless solution | 99.5 | 0.21 | 0.5 |
| 3A4 | Clear, colorless solution | 98.5 | 1.03 | 1.5 |
| 3A5 | Colorless liquid with particulates | 97.9 | 1.53 | 2.1 |
| 3B1 | Clear, light yellow color solution | 99.4 | 0.20 | 0.6 |
| 3B2 | Clear, colorless solution | 99.6 | 0.14 | 0.36 |
| 3B3 | Clear light blue color solution | 99.4 | 0.20 | 0.6 |
| 3B4 | Clear, blue color solution | 98.6 | 0.27 | 1.4 |
| 3B5 | Clear, light blue color solution | 97.9 | 0.40 | 2.1 |
| 3C1 | Clear, yellow color solution | 98.8 | 0.63 | 1.2 |
| 3C2 | Clear, light yellow color solution | 99.3 | 0.28 | 0.7 |
| 3C3 | Clear, light pink color solution | 99.4 | 0.17 | 0.6 |
| 3C4 | Clear, colorless solution | 99.4 | 0.08 | 0.6 |
| 3C5 | Clear, colorless solution | 99.2 | 0.09 | 0.8 |
| 3D1 | Clear, yellow color solution | 94.2 | 1.14 | 5.8 |
| 3D2 | Clear, yellow color solution | 95.9 | 0.69 | 4.1 |
| 3D3 | Clear, colorless solution | 97.2 | 0.28 | 2.8 |
| 3D4 | Clear, colorless solution | 97.7 | 0.29 | 2.3 |
| 3D5 | Clear, colorless solution | 97.9 | 0.31 | 2.1 |
| 3E1 | Clear, yellow color solution | 94.7 | 1.00 | 5.3 |
| 3E2 | Clear, yellow color solution | 95.8 | 0.89 | 4.2 |
| 3E3 | Clear, colorless solution | 97.6 | 0.36 | 2.4 |
| 3E4 | Clear, colorless solution | 97.6 | 0.39 | 2.4 |
| 3E5 | Clear, colorless solution | 97.8 | 0.39 | 2.2 |

Unexpectedly, it was found that increasing the concentration of sodium metabisulfite leads to greater degradation of edaravone in propylene glycol and ethanol. This observation suggests that higher levels of bisulfite may negatively impact product stability, which is counterintuitive for oxygen-sensitive compounds. Such behavior introduces significant challenges in designing antioxidant or stabilizer systems that maintain sink conditions and prevent degradation during storage and use.

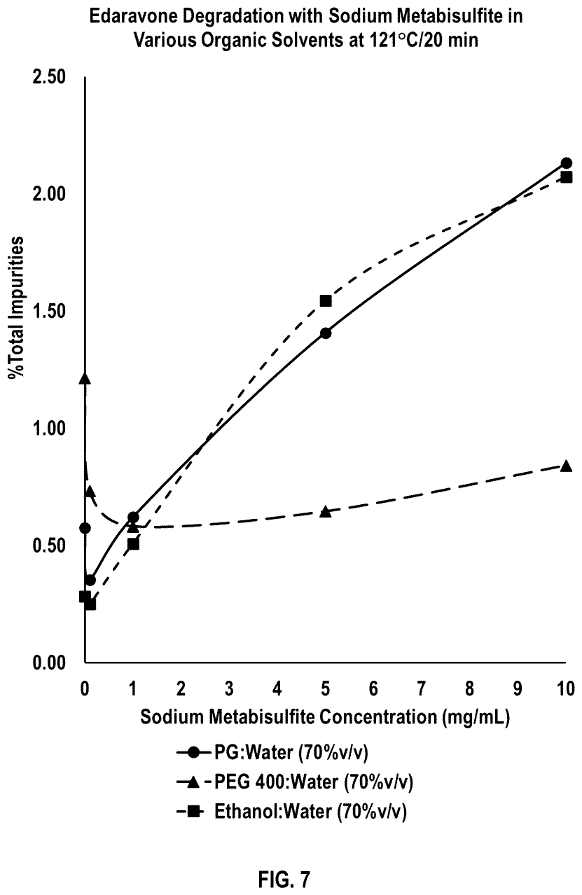

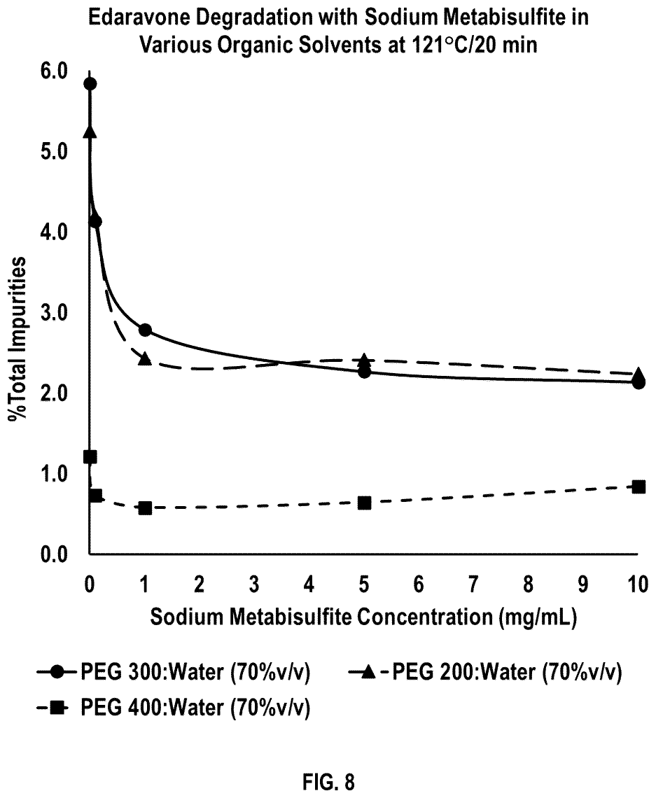

Conversely, polyethylene glycol (PEG) exhibited a different trend: it degraded substantially in the absence of antioxidants but remained relatively stable at low-to-higher bisulfite concentrations. This indicates that edaravone stabilization can be achieved through bisulfite in polyethylene glycol, revealing a unique synergistic effect that both stabilizes the drug and creates a sink condition for degradation products. These findings are illustrated in FIG. 7 and FIG. 8, which shows the relationship between sodium metabisulfite concentration and edaravone degradation in various organic solvent systems at 121° C. for 20 minutes.

Example 4 to 9 describes the composition of liquid formulation of edaravone, prepared using ingredients detailed in Table 13. Where applicable, sucralose, methylparaben, propylparaben, strawberry flavor, sodium lauryl sulfate, and tocopherol were added one-by-one in about 80% of batch required quantity of PEG 400 or propylene glycol and mixed until dissolved under nitrogen overlay. The active ingredient, edaravone was added and mixed under nitrogen overlay until dissolved and clear solution was obtained. The volume of the batches was brought up to batch size using PEG 400 or propylene glycol (where applicable) and mixed to homogeneity. The solution was filtered, filled in to USP type I glass container, and sealed with closure using nitrogen overlay. The stability test results for example 4-9 are provided in Table 14.

TABLE 13
Compositions for edaravone liquid formulations of example 4-9.

| | Example No. (#) - Quantity (mg/mL) | | | | | | | | | |
|---|---|---|---|---|---|---|---|---|---|---|
| Ingredient | #4 | #5 | | #6 | | #7 | #8 | | #9 | |
| Edaravone | 21 mg | 21 | mg | 21 | mg | 21 mg | 21 | mg | 21 | mg |
| Sucralose | — | — | | 2 | mg | — | — | | 2 | mg |
| Methylparaben | — | — | | 2.5 | mg | — | — | | 2.5 | mg |
| Propylparaben | — | — | | 0.25 | mg | — | — | | 0.25 | mg |
| Strawberry flavor | — | — | | 1 | mg | — | — | | 1 | mg |
| Sodium lauryl sulfate | — | — | | — | | — | — | | 1 | mg |
| All-rac-α-Tocopherol | — | 2 | mg | 2 | mg | — | 2 | mg | 2 | mg |

| | | | | | | |
|---|---|---|---|---|---|---|
| PEG 400 | q.s. | q.s. | q.s. | — | — | — |
| | to 1 mL | to 1 mL | to 1 mL | | | |
| Propylene Glycol | — | — | — | q.s. | q.s. | q.s. |
| | | | | to 1 mL | to 1 mL | to 1 mL |

TABLE 14
Stability test results for example 4-9.

| Example | | | | % Maximum | % Total |
|---|---|---|---|---|---|
| No. | Time Point | Visual Description | % Purity | Single Impurity | Impurities |
| 4 | RT/1 M | Clear, colorless solution | 99.8 | 0.13 | 0.2 |
| | RT/2 M | Clear, colorless solution | 99.7 | 0.19 | 0.3 |
| | RT/3 M | Clear, colorless solution | 99.4 | 0.23 | 0.6 |
| | RT/5 M | Clear, colorless solution | 99.3 | 0.35 | 0.7 |
| | RT/6 M | Clear, yellow color solution | 99.3 | 0.38 | 0.7 |
| | RT/12 M | Clear, yellow color solution | 98.6 | 0.84 | 1.4 |
| 5 | RT/1 M | Clear, colorless solution | 99.8 | 0.09 | 0.2 |
| | RT/2 M | Clear, colorless solution | 99.8 | 0.11 | 0.2 |
| | RT/5 M | Clear, colorless solution | 99.5 | 0.23 | 0.5 |
| | RT/6 M | Clear, yellow color solution | 99.5 | 0.25 | 0.5 |
| | RT/12 M | Clear, yellow color solution | 98.6 | 0.80 | 1.4 |
| 6 | RT/1 M | Clear, colorless solution | 99.8 | 0.09 | 0.2 |
| | RT/2 M | Clear, colorless solution | 99.8 | 0.12 | 0.2 |
| | RT/5 M | Clear, colorless solution | 99.5 | 0.23 | 0.5 |
| | RT/6 M | Clear, yellow color solution | 99.4 | 0.26 | 0.6 |
| | RT/12 M | Clear, yellow color solution | 98.9 | 0.58 | 1.1 |
| 7 | RT/1 M | Clear, colorless solution | 99.7 | 0.11 | 0.3 |
| | RT/2 M | Clear, colorless solution | 99.7 | 0.16 | 0.3 |
| | RT/3 M | Clear, colorless solution | 99.6 | 0.17 | 0.4 |
| | RT/5 M | Clear, colorless solution | 99.6 | 0.25 | 0.4 |
| | RT/6 M | Clear, colorless solution | 99.5 | 0.26 | 0.5 |
| | RT/12 M | Clear, light blue color solution | 99.6 | 0.21 | 0.4 |
| 8 | RT/1 M | Clear, colorless solution | 99.9 | 0.07 | 0.1 |
| | RT/2 M | Clear, colorless solution | 99.9 | 0.07 | 0.1 |
| | RT/5 M | Clear, colorless solution | 99.8 | 0.08 | 0.2 |
| | RT/6 M | Clear, colorless solution | 99.8 | 0.09 | 0.2 |
| | RT/12 M | Clear, blue color solution | 99.7 | 0.17 | 0.3 |
| 9 | RT/1 M | Clear, colorless solution | 99.9 | 0.07 | 0.1 |
| | RT/2 M | Clear, colorless solution | 99.9 | 0.10 | 0.1 |
| | RT/5 M | Clear, colorless solution | 99.8 | 0.10 | 0.2 |
| | RT/6 M | Clear, colorless solution | 99.8 | 0.11 | 0.2 |
| | RT/12 M | Clear, blue color solution | 99.5 | 0.23 | 0.5 |

Example 10 to 12 describes the composition of liquid formulation of edaravone, prepared using ingredients detailed in Table 15. Where applicable, sucralose, methylparaben, propylparaben, strawberry flavor, L-cysteine hydrochloride monohydrate, and sodium metabisulfite were added one-by-one in about 80% of batch required quantity of propylene glycol and mixed until dissolved under nitrogen overlay. The active ingredient, edaravone was added and mixed under nitrogen overlay until dissolved and clear solution was obtained. The volume of the batches was brought up to batch size using propylene glycol and mixed to homogeneity. The solution was filtered, filled in to USP type I glass container, and sealed with closure using nitrogen overlay. The stability test results for example 10-12 are provided in Table 16.

TABLE 15
Compositions for edaravone liquid formulations of example 10-12.

| | Example No. (#) - Quantity (mg/mL) | | | |
|---|---|---|---|---|
| Ingredient | | #10 | #11 | #12 |

| | | | | | | |
|---|---|---|---|---|---|---|
| Edaravone | 21 | mg | 21 | mg | 21 | mg |
| Sucralose | 5 | mg | 5 | mg | 5 | mg |
| Methylparaben | 2.5 | mg | 2.5 | mg | 2.5 | mg |
| Propylparaben | 0.25 | mg | 0.25 | mg | 0.25 | mg |
| Strawberry flavor | 1 | mg | 1 | mg | 1 | mg |

| | | | | | |
|---|---|---|---|---|---|
| L-Cysteine | — | 0.25 | mg | 0.25 | mg |
| hydrochloride hydrate | | | | | |

| | | | | | | |
|---|---|---|---|---|---|---|
| Sodium metabisulfite | 2 | mg | 0.25 | mg | 0.25 | mg |

| | | | | |
|---|---|---|---|---|
| Water | — | 0.1 | mL | — |

| | | | |
|---|---|---|---|
| Propylene Glycol | q.s. | q.s. | q.s. |
| | to 1 mL | to 1 mL | to 1 mL |

TABLE 16
Stability test results for example 10-12.

| Example | | | | % Maximum | % Total |
|---|---|---|---|---|---|
| No. | Time Point | Visual Description | % Purity | Single Impurity | Impurities |
| 10 | Initial | Clear, colorless solution | 99.9 | 0.07 | 0.1 |
| | RT/1 M | Clear, colorless solution | 99.8 | 0.09 | 0.2 |
| | RT/5 M | Clear, colorless solution | 99.8 | 0.08 | 0.2 |
| | RT/6 M | Clear, colorless solution | 99.8 | 0.10 | 0.2 |
| | RT/12 M | Clear, very light-yellow color solution | 99.8 | 0.10 | 0.2 |
| 11 | Initial | Clear, colorless solution | 99.9 | 0.07 | 0.1 |
| | RT/1 M | Clear, colorless solution | 99.9 | 0.09 | 0.1 |
| | RT/5 M | Clear, colorless solution | 99.5 | 0.18 | 0.5 |
| | RT/6 M | Clear, colorless solution | 99.3 | 0.22 | 0.7 |
| | RT/12 M | Clear, colorless solution | 99.1 | 0.58 | 0.9 |
| 12 | Initial | Clear, colorless solution | 99.9 | 0.08 | 0.1 |
| | RT/1 M | Clear, colorless solution | 99.9 | 0.09 | 0.1 |
| | RT/5 M | Clear, colorless solution | 99.8 | 0.06 | 0.2 |
| | RT/6 M | Clear, colorless solution | 99.7 | 0.07 | 0.3 |
| | RT/12 M | Clear, colorless solution | 98.9 | 0.80 | 1.1 |

Example 13 to 37 describes the composition of liquid formulation of edaravone, prepared using ingredients detailed in Table 17 to 22. Where applicable, sucralose, methylparaben, propylparaben, strawberry flavor, L-cysteine hydrochloride monohydrate, and sodium metabisulfite were added one-by-one in about 8000 of batch required quantity of propylene glycol and mixed until dissolved under nitrogen overlay. The active ingredient, edaravone was added and mixed until dissolved and clear solution was obtained. Where applicable, required quantity of water was added and mixed to homogeneity. The volume of the batches was brought up to batch size using propylene glycol and mixed to homogeneity. The solution was filtered, filled in to USP type I glass container, and sealed with closure using nitrogen overlay and packaged to protect from light. The stability test results are provided in Table 23.

TABLE 17
Compositions for edaravone liquid formulations of example 13-16.

| | Example No. (#) - Quantity (mg/mL) | | | | | | | |
|---|---|---|---|---|---|---|---|---|
| Ingredient | #13 | | #14 | | #15 | | #16 | |
| Edaravone | 21 | mg | 21 | mg | 21 | mg | 21 | mg |
| Sucralose | 5 | mg | 5 | mg | 5 | mg | 5 | mg |
| Methylparaben | 2.5 | mg | 2.5 | mg | 2.5 | mg | 2.5 | mg |
| Propylparaben | 0.25 | mg | 0.25 | mg | 0.25 | mg | 0.25 | mg |
| Strawberry flavor | 1 | mg | 1 | mg | 1 | mg | 1 | mg |
| Sodium metabisulfite | 0.1 | mg | 0.25 | mg | 0.5 | mg | 1 | mg |

| | | | | |
|---|---|---|---|---|
| Propylene Glycol | q.s. | q.s. | q.s. | q.s. |
| | to 1 mL | to 1 mL | to 1 mL | to 1 mL |

TABLE 18
Compositions for edaravone liquid formulations of example 17-20.

| | Example No. (#) - Quantity (mg/mL) | | | | | | | |
|---|---|---|---|---|---|---|---|---|
| Ingredient | #17 | | #18 | | #19 | | #20 | |
| Edaravone | 21 | mg | 21 | mg | 21 | mg | 21 | mg |
| Sucralose | 5 | mg | 5 | mg | 5 | mg | 5 | mg |
| Methylparaben | 2.5 | mg | 2.5 | mg | 2.5 | mg | 2.5 | mg |
| Propylparaben | 0.25 | mg | 0.25 | mg | 0.25 | mg | 0.25 | mg |
| Strawberry flavor | 1 | mg | 1 | mg | 1 | mg | 1 | mg |

| | | | | | | | |
|---|---|---|---|---|---|---|---|
| Sodium metabisulfite | 0.25 | mg | 0.5 | mg | 0.5 | mg | — |

| | | | | | |
|---|---|---|---|---|---|
| Sodium bisulfite | — | — | — | 0.5 | mg |

| L-cysteine | — | | — | | 0.1 | mg | — |
|---|---|---|---|---|---|---|---|
| hydrochloride hydrate | | | | | | | |
| Water | 0.1 | mL | 0.1 | mL | 0.1 | mL | — |

| | | | | |
|---|---|---|---|---|
| Propylene Glycol | q.s. | q.s. | q.s. | q.s. |
| | to 1 mL | to 1 mL | to 1 mL | to 1 mL |

TABLE 19
Compositions for edaravone liquid formulations of example 21-24.

| | Example No. (#) - Quantity (mg/mL) | | | | | | | |
|---|---|---|---|---|---|---|---|---|
| Ingredient | #21 | | #22 | | #23 | | #24 | |
| Edaravone | 21 | mg | 21 | mg | 21 | mg | 21 | mg |
| Sucralose | 5 | mg | 5 | mg | 5 | mg | 5 | mg |
| Methylparaben | 2.5 | mg | 2.5 | mg | 2.5 | mg | 2.5 | mg |
| Propylparaben | 0.25 | mg | 0.25 | mg | 0.25 | mg | 0.25 | mg |
| Strawberry flavor | 1 | mg | 1 | mg | 1 | mg | 1 | mg |
| Sodium metabisulfite | — | | 0.025 | mg | 0.05 | mg | 0.1 | mg |
| Water | 0.1 | mL | 0.1 | mL | 0.1 | mL | 0.1 | mL |

| | | | | |
|---|---|---|---|---|
| Propylene Glycol | q.s. | q.s. | q.s. | q.s. |
| | to 1 mL | to 1 mL | to 1 mL | to 1 mL |

TABLE 20
Compositions for edaravone liquid formulations of example 25-28.

| | Example No. (#) - Quantity (mg/mL) | | | | | | | |
|---|---|---|---|---|---|---|---|---|
| Ingredient | #25 | | #26 | | #27 | | #28 | |
| Edaravone | 21 | mg | 21 | mg | 21 | mg | 21 | mg |
| Sucralose | 5 | mg | 5 | mg | 5 | mg | 5 | mg |
| Methylparaben | 2.5 | mg | 2.5 | mg | 2.5 | mg | 2.5 | mg |
| Propylparaben | 0.25 | mg | 0.25 | mg | 0.25 | mg | 0.25 | mg |
| Strawberry flavor | 1 | mg | 1 | mg | 1 | mg | 1 | mg |
| Sodium metabisulfite | 0.1 | mg | 0.1 | mg | 0.1 | mg | 0.1 | mg |
| L-cysteine | — | | — | | 0.05 | mg | 0.05 | mg |
| hydrochloride hydrate | | | | | | | | |
| Water | 0.2 | mL | 0.3 | mL | 0.1 | mL | 0.2 | mL |

| | | | | |
|---|---|---|---|---|
| Propylene Glycol | q.s. | q.s. | q.s. | q.s. |
| | to 1 mL | to 1 mL | to 1 mL | to 1 mL |

TABLE 21
Compositions for edaravone liquid formulations of example 29-33.

| | Example No. (#) - Quantity (mg/mL) | | | | | | | | | |
|---|---|---|---|---|---|---|---|---|---|---|
| Ingredient | #29 | | #30 | | #31 | | #32 | | #33 | |
| Edaravone | 21 | mg | 21 | mg | 21 | mg | 21 | mg | 21 | mg |
| Sucralose | 5 | mg | 5 | mg | 5 | mg | 5 | mg | 5 | mg |
| Methylparaben | 2.5 | mg | 2.5 | mg | 2.5 | mg | 2.5 | mg | 2.5 | mg |
| Propylparaben | 0.25 | mg | 0.25 | mg | 0.25 | mg | 0.25 | mg | 0.25 | mg |
| Strawberry flavor | 1 | mg | 1 | mg | 1 | mg | 1 | mg | 1 | mg |
| Sodium metabisulfite | 0.02 | mg | 0.02 | mg | 0.02 | mg | 0.02 | mg | 0.1 | mg |
| L-cysteine | 0.02 | mg | 0.02 | mg | 0.1 | mg | 0.1 | mg | 0.02 | mg |
| hydrochloride hydrate | | | | | | | | | | |
| Water | 0.05 | mL | 0.25 | mL | 0.05 | mL | 0.25 | mL | 0.05 | mL |

| | | | | | |
|---|---|---|---|---|---|
| Propylene Glycol | q.s. | q.s. | q.s. | q.s. | q.s. |
| | to 1 mL | to 1 mL | to 1 mL | to 1 mL | to 1 mL |

TABLE 22
Compositions for edaravone liquid formulations of example 34-37.

| | Example No. (#) - Quantity (mg/mL) | | | | | | | |
|---|---|---|---|---|---|---|---|---|
| Ingredient | #34 | | #35 | | #36 | | #37 | |
| Edaravone | 21 | mg | 21 | mg | 21 | mg | 21 | mg |
| Sucralose | 5 | mg | 5 | mg | 5 | mg | 5 | mg |
| Methylparaben | 2.5 | mg | 2.5 | mg | 2.5 | mg | 2.5 | mg |
| Propylparaben | 0.25 | mg | 0.25 | mg | 0.25 | mg | 0.25 | mg |
| Strawberry flavor | 1 | mg | 1 | mg | 1 | mg | 1 | mg |
| Sodium metabisulfite | 0.1 | mg | 0.1 | mg | 0.1 | mg | 0.05 | mg |
| L-cysteine hydrochloride hydrate | 0.02 | mg | 0.1 | mg | 0.1 | mg | 0.05 | mg |
| Water | 0.25 | mL | 0.05 | mL | 0.25 | mL | 0.1 | mL |

| | | | | |
|---|---|---|---|---|
| Propylene Glycol | q.s. to 1 mL | q.s. to 1 mL | q.s. to 1 mL | q.s. to 1 mL |

TABLE 23
Stability test results for example 13-37.

| Example | | | % | % Maximum | % Total |
|---|---|---|---|---|---|
| No. | Time Point | Visual Description | Purity | Single Impurity | Impurities |
| 13 | Initial | Clear, colorless solution | 99.9 | 0.06 | 0.1 |
| | 40° C./1 M | Clear, very slight yellow color solution | 99.8 | 0.10 | 0.2 |
| | 40° C./2 M | Clear, very slight yellow color solution | 99.7 | 0.10 | 0.3 |
| | 40° C./3 M | Clear, very slight yellow color solution | 99.8 | 0.10 | 0.2 |
| | 40° C./6 M | Clear, slight yellow solution | 99.7 | 0.14 | 0.3 |
| | RT/3 M | Clear, colorless solution | 99.8 | 0.09 | 0.2 |
| | RT/6 M | Clear, colorless solution | 99.8 | 0.11 | 0.2 |
| | RT/10 M | Clear, colorless solution | 99.7 | 0.12 | 0.3 |
| 14 | Initial | Clear, colorless solution | 99.9 | 0.07 | 0.1 |
| | 40° C./1 M | Clear, slight yellow color solution | 99.8 | 0.09 | 0.2 |
| | 40° C./2 M | Clear, slight yellow color solution | 99.8 | 0.10 | 0.2 |
| | 40° C./3 M | Clear, slight yellow color solution | 99.8 | 0.10 | 0.2 |
| | 40° C./6 M | Clear, slight yellow color solution | 99.7 | 0.15 | 0.3 |
| | RT/3 M | Clear, colorless solution | 99.8 | 0.09 | 0.2 |
| | RT/6 M | Clear, colorless solution | 99.7 | 0.11 | 0.3 |
| | RT/10 M | Clear, colorless solution | 99.7 | 0.12 | 0.3 |
| 15 | Initial | Clear, colorless solution | 99.9 | 0.07 | 0.1 |
| | 40° C./1 M | Clear, slight yellow color solution | 99.8 | 0.09 | 0.2 |
| | 40° C./2 M | Clear, slight yellow color solution | 99.8 | 0.10 | 0.2 |
| | 40° C./3 M | Clear, slight yellow color solution | 99.8 | 0.10 | 0.2 |
| | 40° C./6 M | Clear, yellow color solution | 99.6 | 0.16 | 0.4 |
| | RT/3 M | Clear, colorless solution | 99.8 | 0.09 | 0.2 |
| | RT/6 M | Clear, colorless solution | 99.8 | 0.10 | 0.2 |
| | RT/10 M | Clear, very light-yellow color solution | 99.7 | 0.11 | 0.3 |
| 16 | Initial | Clear, colorless solution | 99.9 | 0.07 | 0.1 |
| | 40° C./1 M | Clear, yellow color solution | 99.8 | 0.14 | 0.2 |
| | 40° C./2 M | Clear, yellow color solution | 99.8 | 0.13 | 0.2 |
| | 40° C./3 M | Clear, yellow color solution | 99.7 | 0.15 | 0.3 |
| | 40° C./6 M | Clear, yellow color solution | 99.5 | 0.23 | 0.5 |
| | RT/3 M | Clear, colorless solution | 99.8 | 0.09 | 0.2 |
| | RT/6 M | Clear, light yellow color solution | 99.7 | 0.12 | 0.3 |
| | RT/10 M | Clear, light yellow color solution | 99.7 | 0.16 | 0.3 |
| 17 | Initial | Clear, colorless solution | 99.9 | 0.07 | 0.1 |
| | 40° C./1 M | Clear, very slight yellow color solution | 99.8 | 0.09 | 0.2 |
| | 40° C./2 M | Clear, very slight yellow color solution | 99.8 | 0.10 | 0.2 |
| | 40° C./3 M | Clear, very slight yellow color solution | 99.8 | 0.10 | 0.2 |
| | 40° C./6 M | Clear, yellow color solution | 99.7 | 0.13 | 0.3 |
| | RT/3 M | Clear, colorless solution | 99.8 | 0.08 | 0.2 |
| | RT/6 M | Clear, colorless solution | 99.8 | 0.09 | 0.2 |
| | RT/10 M | Clear, colorless solution | 99.8 | 0.10 | 0.2 |
| 18 | Initial | Clear, colorless solution | 99.9 | 0.07 | 0.1 |
| | 40° C./1 M | Clear, very slight yellow color solution | 99.8 | 0.09 | 0.2 |
| | 40° C./2 M | Clear, very slight yellow color solution | 99.8 | 0.11 | 0.2 |
| | 40° C./3 M | Clear, slight yellow color solution | 99.8 | 0.10 | 0.2 |
| | 40° C./6 M | Clear, yellow color solution | 99.7 | 0.14 | 0.3 |
| | RT/3 M | Clear, colorless solution | 99.8 | 0.08 | 0.2 |
| | RT/6 M | Clear, colorless solution | 99.8 | 0.10 | 0.2 |
| | RT/10 M | Clear, colorless solution | 99.8 | 0.10 | 0.2 |
| 19 | Initial | Clear, colorless solution | 99.9 | 0.07 | 0.1 |
| | 40° C./1 M | Clear, colorless solution | 99.9 | 0.06 | 0.1 |
| | 40° C./2 M | Clear, colorless solution | 99.8 | 0.07 | 0.2 |
| | 40° C./3 M | Clear, colorless solution | 99.7 | 0.07 | 0.3 |
| | 40° C./6 M | Clear, very slight yellow color solution | 99.6 | 0.09 | 0.4 |
| | RT/3 M | Clear, colorless solution | 99.9 | 0.06 | 0.1 |
| | RT/6 M | Clear, colorless solution | 99.9 | 0.07 | 0.1 |
| | RT/10 M | Clear, colorless solution | 99.9 | 0.07 | 0.1 |
| 20 | Initial | Clear, colorless solution | 99.9 | 0.07 | 0.1 |
| | 40° C./1 M | Clear, yellow color solution | 99.8 | 0.11 | 0.2 |
| | 40° C./2 M | Clear, yellow color solution | 99.8 | 0.12 | 0.2 |
| | 40° C./3 M | Clear, yellow color solution | 99.8 | 0.12 | 0.2 |
| | 40° C./6 M | Clear, yellow color solution | 99.6 | 0.19 | 0.4 |
| | RT/3 M | Clear, colorless solution | 99.8 | 0.09 | 0.2 |
| | RT/6 M | Clear, colorless solution | 99.7 | 0.12 | 0.3 |
| | RT/10 M | Clear, light yellow color solution | 99.7 | 0.14 | 0.3 |
| 21 | Initial | Clear, colorless solution | 99.9 | 0.07 | 0.1 |
| | 40° C./1 M | Clear, slight yellow color solution | 99.7 | 0.11 | 0.3 |
| | 40° C./2 M | Clear, slight yellow color solution | 99.6 | 0.15 | 0.4 |
| | 40° C./3 M | Clear, slight yellow color solution | 99.6 | 0.16 | 0.4 |
| | 40° C./6 M | Clear, yellow color solution | 99.3 | 0.28 | 0.7 |
| | RT/3 M | Clear, colorless solution | 99.7 | 0.11 | 0.3 |
| | RT/6 M | Clear, colorless solution | 99.7 | 0.14 | 0.3 |
| | RT/10 M | Clear, colorless solution | 99.6 | 0.16 | 0.4 |
| 22 | Initial | Clear, colorless solution | 99.9 | 0.07 | 0.1 |
| | 40° C./1 M | Clear, colorless solution | 99.8 | 0.07 | 0.2 |
| | 40° C./2 M | Clear, colorless solution | 99.8 | 0.08 | 0.2 |
| | 40° C./3 M | Clear, colorless solution | 99.8 | 0.09 | 0.2 |
| | 40° C./6 M | Clear, colorless solution | 99.7 | 0.11 | 0.3 |
| | RT/3 M | Clear, colorless solution | 99.8 | 0.07 | 0.2 |
| | RT/6 M | Clear, colorless solution | 99.8 | 0.08 | 0.2 |
| | RT/10 M | Clear, colorless solution | 99.8 | 0.09 | 0.2 |
| 23 | Initial | Clear, colorless solution | 99.9 | 0.07 | 0.1 |
| | 40° C./1 M | Clear, colorless solution | 99.8 | 0.07 | 0.2 |
| | 40° C./2 M | Clear, colorless solution | 99.8 | 0.08 | 0.2 |
| | 40° C./3 M | Clear, colorless solution | 99.8 | 0.09 | 0.2 |
| | 40° C./6 M | Clear, colorless solution | 99.7 | 0.11 | 0.3 |
| | RT/3 M | Clear, colorless solution | 99.8 | 0.07 | 0.2 |
| | RT/6 M | Clear, colorless solution | 99.8 | 0.08 | 0.2 |
| | RT/10 M | Clear, colorless solution | 99.8 | 0.10 | 0.2 |
| 24 | Initial | Clear, colorless solution | 99.9 | 0.08 | 0.1 |
| | 40° C./1 M | Clear, colorless solution | 99.8 | 0.07 | 0.2 |
| | 40° C./2 M | Clear, colorless solution | 99.8 | 0.08 | 0.2 |
| | 40° C./3 M | Clear, very slight yellow color solution | 99.8 | 0.09 | 0.2 |
| | 40° C./6 M | Clear, colorless solution | 99.7 | 0.12 | 0.3 |
| | RT/3 M | Clear, colorless solution | 99.8 | 0.07 | 0.2 |
| | RT/6 M | Clear, colorless solution | 99.8 | 0.09 | 0.2 |
| | RT/10 M | Clear, colorless solution | 99.8 | 0.10 | 0.2 |
| 25 | Initial | Clear, colorless solution | 99.9 | 0.08 | 0.1 |
| | 40° C./1 M | Clear, colorless solution | 99.8 | 0.07 | 0.2 |
| | 40° C./2 M | Clear, colorless solution | 99.8 | 0.08 | 0.2 |
| | 40° C./3 M | Clear, very slight yellow color solution | 99.8 | 0.08 | 0.2 |
| | 40° C./6 M | Clear, colorless solution | 99.7 | 0.11 | 0.3 |
| | RT/3 M | Clear, colorless solution | 99.8 | 0.07 | 0.2 |
| | RT/6 M | Clear, colorless solution | 99.8 | 0.08 | 0.2 |
| | RT/10 M | Clear, colorless solution | 99.8 | 0.09 | 0.2 |
| 26 | Initial | Clear, colorless solution | 99.9 | 0.07 | 0.1 |
| | 40° C./1 M | Clear, colorless solution | 99.9 | 0.07 | 0.1 |
| | 40° C./2 M | Clear, colorless solution | 99.8 | 0.08 | 0.2 |
| | 40° C./3 M | Clear, very slight yellow color solution | 99.8 | 0.08 | 0.2 |
| | 40° C./6 M | Clear, colorless solution | 99.7 | 0.12 | 0.3 |
| | RT/3 M | Clear, colorless solution | 99.9 | 0.06 | 0.1 |
| | RT/6 M | Clear, colorless solution | 99.8 | 0.07 | 0.2 |
| | RT/10 M | Clear, colorless solution | 99.8 | 0.09 | 0.2 |
| 27 | Initial | Clear, colorless solution | 99.9 | 0.07 | 0.1 |
| | 40° C./1 M | Clear, colorless solution | 99.8 | 0.06 | 0.2 |
| | 40° C./2 M | Clear, colorless solution | 99.8 | 0.07 | 0.2 |
| | 40° C./3 M | Clear, very slight yellow color solution | 99.8 | 0.07 | 0.2 |
| | 40° C./6 M | Clear, colorless solution | 99.7 | 0.09 | 0.3 |
| | RT/3 M | Clear, colorless solution | 99.8 | 0.07 | 0.2 |
| | RT/6 M | Clear, colorless solution | 99.8 | 0.07 | 0.2 |
| | RT/10 M | Clear, colorless solution | 99.8 | 0.08 | 0.2 |
| 28 | Initial | Clear, colorless solution | 99.9 | 0.07 | 0.1 |
| | 40° C./1 M | Clear, colorless solution | 99.9 | 0.06 | 0.1 |
| | 40° C./2 M | Clear, colorless solution | 99.8 | 0.07 | 0.2 |
| | 40° C./3 M | Clear, very slight yellow color solution | 99.8 | 0.08 | 0.2 |
| | 40° C./6 M | Clear, colorless solution | 99.7 | 0.10 | 0.3 |
| | RT/3 M | Clear, colorless solution | 99.8 | 0.06 | 0.2 |
| | RT/6 M | Clear, colorless solution | 99.8 | 0.07 | 0.2 |
| | RT/10 M | Clear, colorless solution | 99.8 | 0.08 | 0.2 |
| 29 | Initial | Clear, colorless solution | 99.9 | 0.06 | 0.1 |
| | 40° C./2 M | Clear, colorless solution | 99.8 | 0.08 | 0.2 |
| | 40° C./3 M | Clear, colorless solution | 99.7 | 0.09 | 0.3 |
| | 40° C./6 M | Clear, colorless solution | 99.7 | 0.10 | 0.3 |
| | RT/3 M | Clear, colorless solution | 99.9 | 0.06 | 0.1 |
| | RT/6 M | Clear, colorless solution | 99.7 | 0.07 | 0.3 |
| | RT/9 M | Clear, colorless solution | 99.7 | 0.12 | 0.3 |
| 30 | Initial | Clear, colorless solution | 99.9 | 0.06 | 0.1 |
| | 40° C./2 M | Clear, colorless solution | 99.8 | 0.08 | 0.2 |
| | 40° C./3 M | Clear, very slight yellow color solution | 99.8 | 0.09 | 0.2 |
| | 40° C./6 M | Clear, very slight yellow color solution | 99.6 | 0.15 | 0.4 |
| | RT/3 M | Clear, colorless solution | 99.9 | 0.06 | 0.1 |
| | RT/6 M | Clear, colorless solution | 99.8 | 0.07 | 0.2 |
| | RT/9 M | Clear, colorless solution | 99.8 | 0.07 | 0.2 |
| | RT/9 M | Clear, colorless solution | 99.7 | 0.12 | 0.3 |
| 31 | Initial | Clear, colorless solution | 99.9 | 0.05 | 0.1 |
| | 40° C./2 M | Clear, colorless solution | 99.8 | 0.07 | 0.2 |
| | 40° C./3 M | Clear, colorless solution | 99.7 | 0.07 | 0.3 |
| | 40° C./6 M | Clear, colorless solution | 99.6 | 0.11 | 0.4 |
| | RT/3 M | Clear, colorless solution | 99.9 | 0.06 | 0.1 |
| | RT/6 M | Clear, colorless solution | 99.8 | 0.07 | 0.2 |
| | RT/9 M | Clear, colorless solution | 99.7 | 0.11 | 0.3 |
| 32 | Initial | Clear, colorless solution | 99.9 | 0.05 | 0.1 |
| | 40° C./2 M | Clear, colorless solution | 99.8 | 0.07 | 0.2 |
| | 40° C./3 M | Clear, colorless solution | 99.8 | 0.08 | 0.2 |
| | 40° C./6 M | Clear, colorless solution | 99.7 | 0.11 | 0.3 |
| | RT/3 M | Clear, colorless solution | 99.9 | 0.06 | 0.1 |
| | RT/6 M | Clear, colorless solution | 99.8 | 0.07 | 0.2 |
| | RT/9 M | Clear, colorless solution | 99.7 | 0.11 | 0.3 |
| 33 | Initial | Clear, colorless solution | 99.9 | 0.05 | 0.1 |
| | 40° C./2 M | Clear, colorless solution | 99.8 | 0.08 | 0.2 |
| | 40° C./3 M | Clear, very slight yellow color solution | 99.7 | 0.09 | 0.3 |
| | 40° C./6 M | Clear, very slight yellow color solution | 99.7 | 0.12 | 0.3 |
| | RT/3 M | Clear, colorless solution | 99.9 | 0.06 | 0.1 |
| | RT/6 M | Clear, colorless solution | 99.8 | 0.07 | 0.2 |
| | RT/9 M | Clear, colorless solution | 99.6 | 0.10 | 0.4 |
| 34 | Initial | Clear, colorless solution | 99.9 | 0.05 | 0.1 |
| | 40° C./2 M | Clear, colorless solution | 99.8 | 0.08 | 0.2 |
| | 40° C./3 M | Clear, very slight yellow color solution | 99.8 | 0.09 | 0.2 |
| | 40° C./6 M | Clear, slight yellow color solution | 99.7 | 0.13 | 0.3 |
| | RT/3 M | Clear, colorless solution | 99.9 | 0.06 | 0.1 |
| | RT/6 M | Clear, colorless solution | 99.8 | 0.07 | 0.2 |
| | RT/9 M | Clear, colorless solution | 99.8 | 0.06 | 0.2 |
| | RT/9 M | Clear, colorless solution | 99.8 | 0.09 | 0.2 |
| 35 | Initial | Clear, colorless solution | 99.9 | 0.06 | 0.1 |
| | 40° C./2 M | Clear, colorless solution | 99.7 | 0.07 | 0.3 |
| | 40° C./3 M | Clear, colorless solution | 99.7 | 0.08 | 0.3 |
| | 40° C./6 M | Clear, colorless solution | 99.5 | 0.11 | 0.5 |
| | RT/3 M | Clear, colorless solution | 99.9 | 0.06 | 0.1 |
| | RT/6 M | Clear, colorless solution | 99.8 | 0.07 | 0.2 |
| | RT/9 M | Clear, colorless solution | 99.7 | 0.10 | 0.3 |
| 36 | Initial | Clear, colorless solution | 99.9 | 0.05 | 0.1 |
| | 40° C./2 M | Clear, colorless solution | 99.8 | 0.08 | 0.2 |
| | 40° C./3 M | Clear, colorless solution | 99.7 | 0.09 | 0.3 |
| | 40° C./6 M | Clear, colorless solution | 99.7 | 0.10 | 0.3 |
| | RT/3 M | Clear, colorless solution | 99.9 | 0.07 | 0.1 |
| | RT/6 M | Clear, colorless solution | 99.8 | 0.07 | 0.2 |
| | RT/9 M | Clear, colorless solution | 99.6 | 0.13 | 0.4 |
| 37 | Initial | Clear, colorless solution | 99.9 | 0.06 | 0.1 |
| | 40° C./2 M | Clear, colorless solution | 99.8 | 0.07 | 0.2 |
| | 40° C./3 M | Clear, colorless solution | 99.8 | 0.08 | 0.2 |
| | 40° C./6 M | Clear, colorless solution | 99.7 | 0.09 | 0.3 |
| | RT/3 M | Clear, colorless solution | 99.9 | 0.06 | 0.1 |
| | RT/6 M | Clear, colorless solution | 99.7 | 0.07 | 0.3 |
| | RT/9 M | Clear, colorless solution | 99.7 | 0.11 | 0.3 |

Example 38 to 41 describes the composition of liquid formulation of edaravone, prepared using ingredients detailed in Table 24. Where applicable, sucralose, methylparaben, propylparaben, and, strawberry flavor, were added one-by-one in about 80% of batch required quantity of propylene glycol and mixed until dissolved under nitrogen overlay. The active ingredient, edaravone was added and mixed until dissolved and clear solution was obtained. L-cysteine hydrochloride monohydrate, and sodium metabisulfite were dissolved in required quantity of nitrogen sparged water, mixed to dissolve and added to prepared propylene glycol solution. The volume of the batches was brought up to batch size using propylene glycol and mixed to homogeneity. The solution was filtered, filled in to USP type I glass container, and sealed with closure using nitrogen overlay (except for example 41) and packaged to protect from light. The stability test results for example 38-41 are provided in Table 25.

TABLE 24
Compositions for edaravone liquid formulations of example 38-41.

| | Example No. (#) - Quantity (mg/mL) | | | | | | | |
|---|---|---|---|---|---|---|---|---|
| Ingredient | #38 | | #39 | | #40 | | #41* | |
| Edaravone | 21 | mg | 21 | mg | 21 | mg | 21 | mg |
| Sucralose | 5 | mg | 5 | mg | 5 | mg | 5 | mg |
| Methylparaben | 2.5 | mg | 2.5 | mg | 2.5 | mg | 2.5 | mg |
| Propylparaben | 0.25 | mg | 0.25 | mg | 0.25 | mg | 0.25 | mg |
| Strawberry flavor | 1 | mg | 1 | mg | 1 | mg | 1 | mg |
| Sodium metabisulfite | 0.025 | mg | 0.025 | mg | 0.025 | mg | 0.025 | mg |
| L-cysteine hydrochloride hydrate | 0.05 | mg | 0.05 | mg | 0.05 | mg | 0.05 | mg |
| Water | 0.1 | mL | 0.2 | mL | 0.3 | mL | 0.2 | mL |

| | | | | |
|---|---|---|---|---|
| Propylene Glycol | q.s. to 1 mL | q.s. to 1 mL | q.s. to 1 mL | q.s. to 1 mL |

*Without nitrogen overlay

TABLE 25
Stability test results for example 38-41.

| | | | | % Maximum | |
|---|---|---|---|---|---|
| Example | | | | Single | % Total |
| No. | Time Point | Visual Description | % Purity | Impurity | Impurities |
| 38 | Initial | Clear, colorless solution | 99.9 | 0.03 | 0.1 |
| | 40° C./3 M | Clear, slight yellow solution | 99.8 | 0.09 | 0.2 |
| | 40° C./6 M | Clear, slight yellow color solution | 99.6 | 0.14 | 0.4 |
| | RT/3 M | Clear, colorless solution | 99.8 | 0.06 | 0.2 |
| | RT/6 M | Clear, colorless solution | 99.7 | 0.06 | 0.3 |
| | RT/9 M | Clear, colorless solution | 99.8 | 0.08 | 0.2 |
| 39 | Initial | Clear, colorless solution | 99.9 | 0.03 | 0.1 |
| | 40° C./3 M | Clear, slight yellow solution | 99.8 | 0.09 | 0.2 |
| | 40° C./6 M | Clear, slight yellow color solution | 99.4 | 0.20 | 0.6 |
| | RT/3 M | Clear, colorless solution | 99.8 | 0.06 | 0.2 |
| | RT/6 M | Clear, colorless solution | 99.7 | 0.07 | 0.3 |
| | RT/9 M | Clear, colorless solution | 99.8 | 0.11 | 0.2 |
| 40 | Initial | Clear, colorless solution | 99.9 | 0.04 | 0.1 |
| | 40° C./3 M | Clear, slight yellow solution | 99.8 | 0.10 | 0.2 |
| | 40° C./6 M | Clear, slight yellow solution | 99.5 | 0.15 | 0.5 |
| | RT/3 M | Clear, colorless solution | 99.8 | 0.06 | 0.2 |
| | RT/6 M | Clear, colorless solution | 99.7 | 0.07 | 0.3 |
| | RT/9 M | Clear, colorless solution | 99.7 | 0.11 | 0.3 |
| 41 | Initial | Clear, colorless solution | 99.9 | 0.03 | 0.1 |
| | 40° C./3 M | Clear, dark green color solution | 95.7 | 2.76 | 4.3 |
| | 40° C./6 M | Clear, brown-green color solution | 88.3 | 7.78 | 11.7 |
| | RT/3 M | Clear, colorless solution | 99.8 | 0.09 | 0.3 |
| | RT/6 M | Clear, yellow color solution | 99.6 | 0.13 | 0.4 |
| | RT/9 M | Clear, light green color solution | 99.3 | 0.23 | 0.7 |

Example 42 and 43 describes the composition of liquid formulation of edaravone, prepared using ingredients detailed in Table 26. Required quantity of edaravone was added in to batch required quantity of propylene glycol and mixed under nitrogen overlay until dissolved. Volume of the batches was brought up to about 85% using water. Where applicable, sodium bisulfite, L-cysteine hydrochloride monohydrate, sucralose, strawberry flavor, and sodium benzoate were added one by one and mixed under nitrogen overlay until dissolved. pH of the formulation was adjusted to 4 using phosphoric acid and/or sodium hydroxide. Volume of the batches were made up to batch size using water and mixed to homogeneity. The solution was filtered, filled in to USP type I glass container, and sealed with closure using nitrogen overlay and packaged to protect from light. The stability test results for example 42-43 are provided in Table 27.

TABLE 26
Compositions for edaravone liquid formulations of example 42-43.

| | Example No. (#) - Quantity (mg/mL) | | | |
|---|---|---|---|---|
| Ingredient | #42 | | #43 | |
| Edaravone | 21 | mg | 21 | mg |
| Sodium bisulfite | 1 | mg | 1 | mg |
| L-Cysteine Hydrochloride | 0.5 | mg | 0.5 | mg |
| Sucralose | — | | 5 | mg |
| Strawberry flavor | — | | 1 | mg |
| Sodium Benzoate | — | | 1 | mg |
| Sodium hydroxide (0.1N) | q.s. to pH 4 | | q.s. to pH 4 | |
| Phosphoric acid (8.5%) | q.s. to pH 4 | | q.s. to pH 4 | |
| Propylene Glycol | 0.7 | mL | 0.7 | mL |

| | | |
|---|---|---|
| Water | q.s. to 1 mL | q.s. to 1 mL |

TABLE 27
Stability test results for example 42-43.

| | | | | % Maximum | |
|---|---|---|---|---|---|
| Example | | | | Single | % Total |
| No. | Time Point | Visual Description | % Purity | Impurity | Impurities |
| 42 | Initial | Clear, colorless solution | 99.8 | 0.06 | 0.2 |
| | 40° C./2 M | Clear, colorless solution | 98.8 | 0.66 | 1.2 |
| | 40° C./6 M | Clear, yellow color solution | 97.3 | 0.83 | 2.7 |
| | RT/6 M | Clear, colorless solution | 99.1 | 0.43 | 0.9 |
| 43 | Initial | Clear, colorless solution | 99.9 | 0.06 | 0.1 |
| | 40° C./2 M | Clear, colorless solution | 98.6 | 0.79 | 1.4 |
| | 40° C./6 M | Clear, yellow color solution | 97.6 | 1.03 | 2.4 |
| | RT/6 M | Clear, colorless solution | 99.2 | 0.48 | 0.8 |

Example 44 to 51 describes the composition of liquid formulation of edaravone, prepared using ingredients detailed in Table 28. Where applicable, sucralose, methylparaben, propylparaben, and, cherry flavor, were added one-by-one in required quantity of propylene glycol and mixed until dissolved under nitrogen overlay. The active ingredient, edaravone was added and mixed until dissolved and clear solution was obtained. L-cysteine hydrochloride monohydrate, and sodium metabisulfite were dissolved in partial quantity of nitrogen sparged water, mixed to dissolve and added to prepared propylene glycol solution. The volume of the batches was brought up to batch size using water and mixed to homogeneity. The solution was filtered, filled in to containers, sealed with closure with nitrogen overlay where applicable, and packaged to protect from light. Packaging components, nitrogen overlay during closing containers and orientation of containers during stability for example 44-51 are provided in Table 29. Stability test results are provided in Table 30.

TABLE 28
Compositions for edaravone liquid formulations of example 44-51.

| | Example No. (#) - Quantity (mg/mL) | | | |
|---|---|---|---|---|
| Ingredient | #44 to #47 | | #48 to #51 | |
| Edaravone | 21 | mg | 21 | mg |
| Sucralose | 5 | mg | 5 | mg |
| Methylparaben | 2.5 | mg | 2.5 | mg |
| Propylparaben | 0.25 | mg | 0.25 | mg |
| Cherry flavor | 1 | mg | 1 | mg |
| Sodium metabisulfite | 1 | mg | 0.1 | mg |
| L-cysteine hydrochloride hydrate | 0.5 | mg | 0.05 | mg |
| Propylene glycol | 0.8 | mL | 0.8 | mL |

| | | |
|---|---|---|
| Water | q.s. to 1 mL | q.s. to 1 mL |

TABLE 29
Packaging for edaravone liquid formulation of example 44-51.

| | | | Nitrogen | Orientation |
|---|---|---|---|---|
| Example | | | overlay | during stability |
| No. | Primary Packaging | Fill Volume | (Yes/ No) | testing |
| 44 | 10 CC Glass bottle with screw cap | ~7.5 mL | No | Horizontal |
| 45 | 10 CC Glass bottle with screw cap | ~7.5 mL | Yes | Horizontal |
| 46 | 10 CC HDPE bottle with screw cap | ~7.5 mL | No | Upright |
| 47 | 10 CC HDPE bottle with screw cap | ~7.5 mL | Yes | Horizontal |
| 48 | 10 CC Glass bottle with screw cap | ~7.5 mL | No | Horizontal |
| 49 | 10 CC Glass bottle with screw cap | ~7.5 mL | Yes | Horizontal |
| 50 | 10 CC HDPE bottle with screw cap | ~7.5 mL | No | Upright |
| 51 | 10 CC HDPE bottle with screw cap | ~7.5 mL | Yes | Horizontal |

TABLE 30
Stability test results for example 44-51.

| | | | | % Maximum | |
|---|---|---|---|---|---|
| Example | | | | Single | % Total |
| No. | Time Point | Visual Description | % Purity | Impurity | Impurities |
| 44 | Initial | Clear, colorless solution | 99.9 | 0.06 | 0.1 |
| | 40° C./1 M | Clear, colorless solution | 99.4 | 0.22 | 0.6 |
| | 40° C./2 M | Clear, slight yellow color solution | 98.8 | 0.45 | 1.2 |
| | 40° C./6 M | Clear, very light-yellow color solution | 97.8 | 0.98 | 2.2 |
| | RT/6 M | Clear, colorless solution | 99.3 | 0.21 | 0.7 |
| 45 | Initial | Clear, colorless solution | 99.9 | 0.06 | 0.1 |
| | 40° C./1 M | Clear, colorless solution | 99.4 | 0.22 | 0.6 |
| | 40° C./2 M | Clear, colorless solution | 98.9 | 0.40 | 1.1 |
| | 40° C./6 M | Clear, very light yellow color solution | 98.4 | 0.32 | 1.6 |
| | RT/6 M | Clear, colorless solution | 99.5 | 0.13 | 0.5 |
| 46 | Initial | Clear, colorless solution | 99.9 | 0.06 | 0.1 |
| | 40° C./1 M | Clear, colorless solution | 99.3 | 0.23 | 0.7 |
| | 40° C./2 M | Clear, slight yellow color solution | 98.8 | 0.46 | 1.2 |
| | 40° C./6 M | Clear, very light yellow color solution | 97.7 | 1.02 | 2.3 |
| | RT/6 M | Clear, colorless solution | 99.3 | 0.21 | 0.7 |
| 47 | Initial | Clear, colorless solution | 99.9 | 0.06 | 0.1 |
| | 40° C./6 M | Clear, very light yellow color solution | 98.1 | 0.67 | 1.9 |
| | RT/6 M | Clear, colorless solution | 99.4 | 0.17 | 0.6 |
| 48 | Initial | Clear, colorless solution | 99.9 | 0.06 | 0.1 |
| | 40° C./1 M | Clear, colorless solution | 99.7 | 0.19 | 0.3 |
| | 40° C./2 M | Clear, slight yellow color solution | 99.4 | 0.39 | 0.6 |
| | 40° C./6 M | Clear, yellow color solution | 98.3 | 1.25 | 1.7 |
| | RT/6 M | Clear, colorless solution | 99.7 | 0.15 | 0.3 |
| 49 | Initial | Clear, colorless solution | 99.9 | 0.06 | 0.1 |
| | 40° C./1 M | Clear, colorless solution | 99.8 | 0.09 | 0.2 |
| | 40° C./2 M | Clear, colorless solution | 99.7 | 0.12 | 0.3 |
| | 40° C./6 M | Clear, yellow color solution | 99.6 | 0.24 | 0.4 |
| | RT/6 M | Clear, colorless solution | 99.8 | 0.10 | 0.2 |
| 50 | Initial | Clear, colorless solution | 99.9 | 0.06 | 0.1 |
| | 40° C./1 M | Clear, colorless solution | 99.7 | 0.19 | 0.3 |
| | 40° C./2 M | Clear, slight yellow color solution | 99.4 | 0.37 | 0.6 |
| | 40° C./6 M | Clear, yellow color solution | 98.4 | 1.20 | 1.6 |
| | RT/6 M | Clear, colorless solution | 99.7 | 0.16 | 0.3 |
| 51 | Initial | Clear, colorless solution | 99.9 | 0.06 | 0.1 |
| | 40° C./6 M | Clear, yellow color solution | 98.8 | 0.89 | 1.2 |
| | RT/6 M | Clear, colorless solution | 99.8 | 0.12 | 0.2 |

Examples 52-57 describe the composition of liquid formulation of edaravone, prepared using ingredients (vehicle and excipients) detailed in Table 31. Edaravone was dissolved in vehicle at 21 mg/mL. The prepared solution was filled in 2R USP Type I clear tubular glass vial with 1 mL fill volume. Required quantity of excipients, shown in Table 31, were added in the vial and mixed on vortex mixer to homogeneity. Clear, colorless solution was obtained for all samples prepared. The vials were closed using 13 mm stopper with nitrogen overlay and sealed with aluminum crimp seal.

Example 58 describes the composition of suspension formulation of edaravone prepared using ingredients detailed in Table 32, which is also comparable to example 26 given in the U.S. Pat. No. 10,987,341 without use of polyvinyl alcohol. Required quantity of sodium bisulfite, L-cysteine hydrochloride hydrate, simethicone emulsion, sorbitol, xanthan gum, edaravone particles were dissolved or dispersed in about 9000 of batch required water and mixed to homogeneity. pH was adjusted to 4.1 using dilute phosphoric acid and/or sodium hydroxide. Volume was made up to batch size using water and mixed to homogeneity. The prepared suspension was filled in 2R USP type I clear tubular glass vial with 1 mL of fill volume, closed using 13 mm stopper with nitrogen overlay and sealed with aluminum crimp seal.

TABLE 31
Compositions for edaravone liquid formulations of example 52-57.

| | Example | | Excipient amount |
|---|---|---|---|
| Vehicle | No. | Added excipient | added |
| PEG 400 | 52A | Sodium hydroxide (50% w/v) | 4.8 mg (9.6 μL) |
| | 52B | Hydrochloric acid (1N) | 30 μL |
| | 52C | Lactic acid (90% w/v) | 9 mg (10 μL) |
| | 52D | Citric acid (50% w/v) | 6.25 mg (12.5 μL) |
| | 52E | Phosphoric acid (85% w/v) | 2.76 μL |
| | 52F | Disodium edetate (2% w/v) | 1 mg (50 μL) |
| | 52G | — | — |
| PEG 400 (70% v/v in water, | 53A | Sodium hydroxide (50% w/v) | 4.8 mg (9.6 μL) |
| 0.1 mg/mL sodium | 53B | Hydrochloric acid (1N) | 30 μL |
| metabisulfite) | 53C | Lactic acid (90% w/v) | 9 mg (10 μL) |
| | 53D | Citric acid (50% w/v) | 6.25 mg (12.5 μL) |
| | 53E | Phosphoric acid (85% w/v) | 2.76 μL |
| | 53F | Disodium edetate (2% w/v) | 1 mg (50 μL) |
| | 53G | — | — |
| Propylene Glycol (PG) | 54A | Sodium hydroxide (50% w/v) | 4.8 mg (9.6 μL) |
| | 54B | Hydrochloric acid (1N) | 30 μL |
| | 54C | Lactic acid (90% w/v) | 9 mg (10 μL) |
| | 54D | Citric acid (50% w/v) | 6.25 mg (12.5 μL) |
| | 54E | Phosphoric acid (85% w/v) | 2.76 μL |
| | 54F | Disodium edetate (2% w/v) | 1 mg (50 μL) |
| | 54G | — | — |
| PG (70% v/v in water, 0.1 | 55A | Sodium hydroxide (50% w/v) | 4.8 mg (9.6 μL) |
| mg/mL sodium | 55B | Hydrochloric acid (1N) | 30 μL |
| metabisulfite) | 55C | Lactic acid (90% w/v) | 9 mg (10 μL) |
| | 55D | Citric acid (50% w/v) | 6.25 mg (12.5 μL) |
| | 55E | Phosphoric acid (85% w/v) | 2.76 μL |
| | 55F | Disodium edetate (2% w/v) | 1 mg (50 μL) |
| | 55G | — | — |
| PEG 400 (50% v/v in | 56A | Sodium hydroxide (50% w/v) | 4.8 mg (9.6 μL) |
| Glycerin) | 56B | Hydrochloric acid (1N) | 30 μL |
| | 56C | Lactic acid (90% w/v) | 9 mg (10 μL) |
| | 56D | Citric acid (50% w/v) | 6.25 mg (12.5 μL) |
| | 56E | Phosphoric acid (85% w/v) | 2.76 μL |
| | 56F | Disodium edetate (2% w/v) | 1 mg (50 μL) |
| | 56G | Sodium metabisulfite (10% w/v) | 1 mg (10 μL) |
| | 56H | — | — |
| PG (40% v/v in Glycerin) | 57A | Sodium hydroxide (50% w/v) | 4.8 mg (9.6 μL) |
| | 57B | Hydrochloric acid (1N) | 30 μL |
| | 57C | Lactic acid (90% w/v) | 9 mg (10 μL) |
| | 57D | Citric acid (50% w/v) | 6.25 mg (12.5 μL) |
| | 57E | Phosphoric acid (85% w/v) | 2.76 μL |
| | 57F | Disodium edetate (2% w/v) | 1 mg (50 μL) |
| | 57G | Sodium metabisulfite (10% w/v) | 1 mg (10 μL) |
| | 57H | — | — |
| | 57I | Sodium metabisulfite (10% w/v); | 1 mg (10 μL); |
| | | Phosphoric acid (85% w/v) | 2.76 μL |
| | 57J | Sodium metabisulfite (10% w/v); | 1 mg (10 μL); |
| | | Citric acid (50% w/v) | 5 mg (10 μL) |

TABLE 32
Compositions for edaravone suspension
formulations of example 58.

| Ingredient | Example No. 58 - Quantity (mg/mL) | |
|---|---|---|
| Edaravone | 21 | mg |
| Sodium bisulfite | 1 | mg |
| L-cysteine hydrochloride hydrate | 0.5 | mg |
| Simethicone emulsion | 0.5 | mg |
| Sorbitol | 400 | mg |
| Xanthan gum | 3 | mg |

| | |
|---|---|
| Phosphoric acid (8.5%) | q.s. to pH 4.1 |
| Sodium hydroxide (0.1N) | q.s. to pH 4.1 |
| Purified water | q.s. to 1 mL |

TABLE 33
Stability test results for example 52-58.

| Example | | | | % Maximum | % Total |
|---|---|---|---|---|---|
| No. | Time Point | Visual Description | % Purity | Single Impurity | Impurities |
| 52A | 60° C./1 W | Clear, light pink color solution | 99.6 | 0.10 | 0.4 |
| | 60° C./2 W | Clear, pink color solution | 99.4 | 0.11 | 0.6 |
| | 60° C./1 M | Clear, pink color solution | 99.1 | 0.16 | 0.9 |
| 52B | 60° C./1 W | Clear, colorless solution | 99.1 | 0.30 | 0.9 |
| | 60° C./2 W | Clear, light yellow color solution | 98.3 | 0.74 | 1.7 |
| | 60° C./1 M | Clear, light yellow color solution | 96.7 | 1.82 | 3.3 |
| 52C | 60° C./1 W | Clear, yellow color solution | 99.5 | 0.24 | 0.5 |
| | 60° C./2 W | Clear, yellow color solution | 99.3 | 0.40 | 0.7 |
| | 60° C./1 M | Clear, yellow color solution | 99.0 | 0.53 | 1.0 |
| 52D | 60° C./1 W | Clear, colorless solution | 99.7 | 0.14 | 0.3 |
| | 60° C./2 W | Clear, light yellow color solution | 99.5 | 0.16 | 0.5 |
| | 60° C./1 M | Clear, light yellow color solution | 99.4 | 0.21 | 0.6 |
| 52E | 60° C./1 W | Clear, colorless solution | 99.6 | 0.14 | 0.4 |
| | 60° C./2 W | Clear, light yellow color solution | 99.4 | 0.17 | 0.6 |
| | 60° C./1 M | Clear, light yellow color solution | 99.1 | 0.28 | 0.9 |
| 52F | 60° C./1 W | Clear, yellow color solution | 99.5 | 0.25 | 0.5 |
| | 60° C./2 W | Clear, yellow color solution | 99.4 | 0.28 | 0.6 |
| | 60° C./1 M | Clear, light brown color solution | 99.1 | 0.38 | 0.9 |
| 52G | 60° C./1 W | Clear, yellow color solution | 99.4 | 0.30 | 0.6 |
| | 60° C./2 W | Clear, yellow color solution | 99.3 | 0.36 | 0.7 |
| | 60° C./1 M | Clear, yellow color solution | 99.0 | 0.51 | 1.0 |
| 53A | 60° C./1 W | Clear, light pink color solution | 99.7 | 0.08 | 0.3 |
| | 60° C./2 W | Clear, light pink color solution | 99.6 | 0.08 | 0.4 |
| | 60° C./1 M | Clear, light pink color solution | 99.6 | 0.08 | 0.4 |
| 53B | 60° C./1 W | Clear, colorless solution | 99.0 | 0.32 | 1.0 |
| | 60° C./2 W | Clear, colorless solution | 98.3 | 0.66 | 1.7 |
| | 60° C./1 M | Clear, colorless solution | 95.5 | 2.65 | 4.5 |
| 53C | 60° C./1 W | Clear, colorless solution | 99.5 | 0.15 | 0.5 |
| | 60° C./2 W | Clear, colorless solution | 99.5 | 0.17 | 0.5 |
| | 60° C./1 M | Clear, colorless solution | 99.3 | 0.25 | 0.7 |
| 53D | 60° C./1 W | Clear, colorless solution | 99.6 | 0.12 | 0.4 |
| | 60° C./2 W | Clear, colorless solution | 99.5 | 0.14 | 0.5 |
| | 60° C./1 M | Clear, colorless solution | 99.4 | 0.21 | 0.6 |
| 53E | 60° C./1 W | Clear, colorless solution | 99.4 | 0.27 | 0.6 |
| | 60° C./2 W | Clear, colorless solution | 99.2 | 0.33 | 0.8 |
| | 60° C./1 M | Clear, colorless solution | 98.3 | 0.57 | 1.7 |
| 53F | 60° C./1 W | Clear, yellow color solution | 99.6 | 0.13 | 0.4 |
| | 60° C./2 W | Clear, yellow color solution | 99.6 | 0.16 | 0.4 |
| | 60° C./1 M | Clear, yellow color solution | 99.5 | 0.22 | 0.5 |
| 53G | 60° C./1 W | Clear, yellow color solution | 99.6 | 0.15 | 0.4 |
| | 60° C./2 W | Clear, yellow color solution | 99.5 | 0.18 | 0.5 |
| | 60° C./1 M | Clear, yellow color solution | 99.3 | 0.28 | 0.7 |
| 54A | 60° C./1 W | Clear, yellow color solution | 99.5 | 0.20 | 0.5 |
| | 60° C./2 W | Clear, yellow color solution | 99.2 | 0.26 | 0.8 |
| | 60° C./1 M | Clear, light brown color solution | 98.8 | 0.38 | 1.2 |
| 54B | 60° C./1 W | Clear, colorless solution | 99.7 | 0.07 | 0.3 |
| | 60° C./2 W | Clear, colorless solution | 99.5 | 0.14 | 0.5 |
| | 60° C./1 M | Clear, colorless solution | 99.0 | 0.36 | 1.0 |
| 54C | 60° C./1 W | Clear, colorless solution | 99.7 | 0.11 | 0.3 |
| | 60° C./2 W | Clear, colorless solution | 99.7 | 0.12 | 0.3 |
| | 60° C./1 M | Clear, colorless solution | 99.6 | 0.12 | 0.4 |
| 54D | 60° C./1 W | Clear, colorless solution | 99.7 | 0.08 | 0.3 |
| | 60° C./2 W | Clear, colorless solution | 99.7 | 0.08 | 0.3 |
| | 60° C./1 M | Clear, colorless solution | 99.6 | 0.10 | 0.4 |
| 54E | 60° C./1 W | Clear, colorless solution | 99.7 | 0.08 | 0.3 |
| | 60° C./2 W | Clear, colorless solution | 99.6 | 0.09 | 0.4 |
| | 60° C./1 M | Clear, colorless solution | 99.5 | 0.10 | 0.5 |
| 54F | 60° C./1 W | Clear, light brown color solution | 99.1 | 0.33 | 0.9 |
| | 60° C./2 W | Clear, light brown color solution | 99.0 | 0.35 | 1.0 |
| | 60° C./1 M | Clear, brown color solution | 98.6 | 0.42 | 1.4 |
| 54G | 60° C./1 W | Clear light green color solution | 99.4 | 0.16 | 0.6 |
| | 60° C./2 W | Clear light green color solution | 99.3 | 0.21 | 0.7 |
| | 60° C./1 M | Clear green color solution | 99.1 | 0.24 | 0.9 |
| 55A | 60° C./1 W | Clear, colorless solution | 99.3 | 0.29 | 0.7 |
| | 60° C./2 W | Clear, light yellow color solution | 98.6 | 0.38 | 1.4 |
| | 60° C./1 M | Clear, light yellow color solution | 98.4 | 0.58 | 1.6 |
| 55B | 60° C./1 W | Clear, colorless solution | 99.4 | 0.13 | 0.6 |
| | 60° C./2 W | Clear, colorless solution | 98.8 | 0.26 | 1.2 |
| | 60° C./1 M | Clear, colorless solution | 97.2 | 1.68 | 2.8 |
| 55C | 60° C./1 W | Clear, colorless solution | 99.7 | 0.06 | 0.3 |
| | 60° C./2 W | Clear, colorless solution | 99.6 | 0.07 | 0.4 |
| | 60° C./1 M | Clear, colorless solution | 99.4 | 0.10 | 0.6 |
| 55D | 60° C./1 W | Clear, colorless solution | 99.7 | 0.06 | 0.3 |
| | 60° C./2 W | Clear, colorless solution | 99.7 | 0.07 | 0.3 |
| | 60° C./1 M | Clear, colorless solution | 99.5 | 0.10 | 0.5 |
| 55E | 60° C./1 W | Clear, colorless solution | 99.7 | 0.06 | 0.3 |
| | 60° C./2 W | Clear, colorless solution | 99.5 | 0.09 | 0.5 |
| | 60° C./1 M | Clear, colorless solution | 98.8 | 0.63 | 1.2 |
| 55F | 60° C./1 W | Clear, very light-yellow color solution | 99.7 | 0.12 | 0.3 |
| | 60° C./2 W | Clear, very light-yellow color solution | 99.6 | 0.15 | 0.4 |
| | 60° C./1 M | Clear, very light-yellow color solution | 99.5 | 0.25 | 0.5 |
| 55G | 60° C./1 W | Clear, very light-yellow color solution | 99.7 | 0.10 | 0.3 |
| | 60° C./2 W | Clear, very light-yellow color solution | 99.7 | 0.11 | 0.3 |
| | 60° C./1 M | Clear, very light-yellow color solution | 99.6 | 0.16 | 0.4 |
| 56A | 60° C./1 W | Clear, pink color solution | 99.5 | 0.14 | 0.5 |
| | 60° C./2 W | Clear, pink color solution | 99.4 | 0.16 | 0.6 |
| | 60° C./1 M | Clear, pink color solution | 99.3 | 0.18 | 0.7 |
| 56B | 60° C./1 W | Clear, very light-yellow color solution | 99.5 | 0.18 | 0.5 |
| | 60° C./2 W | Clear, very light-yellow color solution | 99.2 | 0.18 | 0.8 |
| | 60° C./1 M | Clear, very light-yellow color solution | 98.7 | 0.38 | 1.3 |
| 56C | 60° C./1 W | Clear, very light-yellow color solution | 99.6 | 0.18 | 0.4 |
| | 60° C./2 W | Clear, very light-yellow color solution | 99.5 | 0.19 | 0.5 |
| | 60° C./1 M | Clear, very light-yellow color solution | 99.6 | 0.19 | 0.4 |
| 56D | 60° C./1 W | Clear, very light-yellow color solution | 99.6 | 0.16 | 0.4 |
| | 60° C./2 W | Clear, very light-yellow color solution | 99.5 | 0.17 | 0.5 |
| | 60° C./1 M | Clear, very light-yellow color solution | 99.5 | 0.17 | 0.5 |
| 56E | 60° C./1 W | Clear, very light-yellow color solution | 99.6 | 0.15 | 0.4 |
| | 60° C./2 W | Clear, very light-yellow color solution | 99.5 | 0.15 | 0.5 |
| | 60° C./1 M | Clear, very light-yellow color solution | 99.4 | 0.15 | 0.6 |
| 56F | 60° C./1 W | Clear, yellow color solution | 99.4 | 0.20 | 0.6 |
| | 60° C./2 W | Clear, yellow color solution | 99.2 | 0.23 | 0.8 |
| | 60° C./1 M | Clear, yellow color solution | 99.1 | 0.26 | 0.9 |
| 56G | 60° C./1 W | Clear, gray color solution | 99.7 | 0.13 | 0.3 |
| | 60° C./2 W | Clear, gray color solution | 99.6 | 0.13 | 0.4 |
| | 60° C./1 M | Clear, light green color solution | 99.4 | 0.14 | 0.6 |
| 56H | 60° C./1 W | Clear, gray color solution | 99.5 | 0.21 | 0.5 |
| | 60° C./2 W | Clear, gray color solution | 99.3 | 0.22 | 0.7 |
| | 60° C./1 M | Clear, green color solution | 99.2 | 0.28 | 0.8 |
| 57A | 60° C./1 W | Clear, pink color solution | 99.3 | 0.17 | 0.7 |
| | 60° C./2 W | Clear, pink color solution | 99.2 | 0.22 | 0.8 |
| | 60° C./1 M | Clear, pink color solution | 98.9 | 0.27 | 1.1 |
| 57B | 60° C./1 W | Clear, colorless solution | 99.6 | 0.10 | 0.4 |
| | 60° C./2 W | Clear, colorless solution | 99.4 | 0.12 | 0.6 |
| | 60° C./1 M | Clear, colorless solution | 99.0 | 0.33 | 1.0 |
| 57C | 60° C./1 W | Clear, colorless solution | 99.7 | 0.08 | 0.3 |
| | 60° C./2 W | Clear, colorless solution | 99.7 | 0.08 | 0.3 |
| | 60° C./1 M | Clear, colorless solution | 99.6 | 0.09 | 0.4 |
| 57D | 60° C./1 W | Clear, colorless solution | 99.7 | 0.08 | 0.3 |
| | 60° C./2 W | Clear, colorless solution | 99.7 | 0.08 | 0.3 |
| | 60° C./1 M | Clear, colorless solution | 99.6 | 0.09 | 0.4 |
| 57E | 60° C./1 W | Clear, colorless solution | 99.7 | 0.07 | 0.3 |
| | 60° C./2 W | Clear, colorless solution | 99.6 | 0.08 | 0.4 |
| | 60° C./1 M | Clear, colorless solution | 99.5 | 0.08 | 0.5 |
| 57F | 60° C./1 W | Clear, yellow color solution | 99.4 | 0.13 | 0.6 |
| | 60° C./2 W | Clear, yellow color solution | 99.2 | 0.14 | 0.8 |
| | 60° C./1 M | Clear, yellow color solution | 99.1 | 0.18 | 0.9 |
| 57G | 60° C./1 W | Clear, greyish blue color solution | 99.6 | 0.08 | 0.4 |
| | 60° C./2 W | Clear, greyish blue color solution | 99.5 | 0.10 | 0.5 |
| | 60° C./1 M | Clear, greyish blue color solution | 99.4 | 0.13 | 0.6 |
| 57H | 60° C./1 W | Clear, light blue color solution | 99.5 | 0.12 | 0.5 |
| | 60° C./2 W | Clear, light blue color solution | 99.4 | 0.12 | 0.6 |
| | 60° C./1 M | Clear, light blue color solution | 99.4 | 0.14 | 0.6 |
| 57I | 60° C./1 W | Clear, colorless solution | 99.8 | 0.07 | 0.2 |
| | 60° C./2 W | Clear, colorless solution | 99.6 | 0.09 | 0.4 |
| | 60° C./1 M | Clear, colorless solution | 99.5 | 0.08 | 0.5 |
| 57J | 60° C./1 W | Clear, colorless solution | 99.7 | 0.08 | 0.3 |
| | 60° C./2 W | Clear, colorless solution | 99.6 | 0.08 | 0.4 |
| | 60° C./1 M | Clear, light yellow color solution | 99.5 | 0.08 | 0.5 |
| 58 | 60° C./1 W | Off-White suspension (after shaking | 99.3 | 0.51 | 0.7 |
| | | for ~30 sec.) | | | |
| | 60° C./2 W | Off-White suspension (after shaking | 99.0 | 0.69 | 1.0 |
| | | for ~30 sec.) | | | |
| | 60° C./1 M | Off-White suspension (after shaking | 98.4 | 0.75 | 1.6 |
| | | for ~30 sec.) | | | |

Stability test results from examples 52-57 suggest that edaravone may be compatible with several excipients. While some excipients show higher impurities, they may be stable at different or lower concentrations. Concentration of excipients selected in this study was arbitrary to evaluate the compatibility of edaravone with excipient in given vehicle.

Example 59 to 63 describes the composition of liquid formulation of edaravone, prepared using ingredients detailed in Table 34. Where applicable, required quantity of sodium metabisulfite, and citric acid were added one-by-one in about 80% of batch required quantity of PEG 400 or propylene glycol and mixed to homogeneity. Required quantity of edaravone was added and mixed under nitrogen overlay until dissolved and clear solution was obtained. Required quantity of glycerin and water (where applicable) were added and mixed to homogeneity. The volume of the batches was brought up to batch size using propylene glycol or PEG 400 and mixed to homogeneity. The solution was filled in to USP type I glass container and sealed with closure using nitrogen overlay. The stability test results for example 59-63 are provided in Table 35.

TABLE 34
Compositions for edaravone liquid formulations of example 59-63.

| | Example No. (#) - Quantity (mg/mL) | | | | | | | |
|---|---|---|---|---|---|---|---|---|
| Ingredient | #59 | | #60 | #61 | #62 | | #63 | |
| Edaravone | 21 | mg | 21 mg | 21 mg | 21 | mg | 21 | mg |
| Citric acid monohydrate | 5 mg | | 5 mg | 5 mg | 5 mg | | 5 mg | |
| (50% w/v in water) | (10 μL) | | (10 μL) | (10 μL) | (10 μL) | | (10 μL) | |
| Sodium metabisulfite | 0.1 mg | | — | 0.1 mg | 0.1 mg | | 0.1 mg | |
| (2% w/v in water) | (5 μL) | | | (5 μL) | (5 μL) | | (5 μL) | |
| Water | 0.1 | mL | — | — | 0.1 | mL | 0.1 | mL |

| | | | | | | |
|---|---|---|---|---|---|---|
| Glycerin | — | — | — | — | 0.3 | mL |

| | | | | | |
|---|---|---|---|---|---|
| Polyethylene glycol 400 | q.s. to 1 mL | — | — | — | — |
| Propylene glycol | — | q.s. to 1 mL | q.s. to 1 mL | q.s. to 1 mL | q.s. to 1 mL |
| Final pH (post 20x dilution) | 3.4 | 3.2 | 3.3 | 3.3 | 3.3 |

TABLE 35
Stability test results for example 59-63.

| Example | | | | % Maximum | % Total |
|---|---|---|---|---|---|
| No. | Time Point | Visual Description | % Purity | Single Impurity | Impurities |
| 59 | Initial | Clear, colorless solution | 99.9 | 0.04 | 0.1 |
| | 40° C./1 M | Clear, colorless solution | 99.6 | 0.18 | 0.4 |
| 60 | Initial | Clear, colorless solution | 99.9 | 0.04 | 0.1 |
| | 40° C./1 M | Clear, colorless solution | 99.5 | 0.22 | 0.5 |
| 61 | Initial | Clear, colorless solution | 99.9 | 0.04 | 0.1 |
| | 40° C./1 M | Clear, colorless solution | 99.6 | 0.11 | 0.4 |
| 62 | Initial | Clear, colorless solution | 99.9 | 0.04 | 0.1 |
| | 40° C./1 M | Clear, colorless solution | 99.7 | 0.09 | 0.3 |
| 63 | Initial | Clear, colorless solution | 99.9 | 0.04 | 0.1 |
| | 40° C./1 M | Clear, colorless solution | 99.7 | 0.08 | 0.3 |

Example 64 and 65 describes the composition of liquid formulation of edaravone, prepared using ingredients detailed in Table 36. In first step, citrate buffer was prepared by dissolving required quantity of citric acid, and sodium metabisulfite in about 90% of the required quantity of water previously sparged with nitrogen. pH was slowly adjusted to target using dilute sodium hydroxide. Volume of buffer was made up to the target, if required. In separate vessels, required quantity of edaravone was added in about 800 of batch required quantity of PEG 400 and mixed under nitrogen overlay until dissolved and clear solution was obtained. Then previously prepared citrate buffer was added to edaravone solution and mixed to homogeneity. The volume of the batches was brought up to batch size using PEG 400 and mixed to homogeneity. The solution was filled in to USP type I glass container and sealed with closure using nitrogen overlay. The stability test results for example 64-65 are provided in Table 37.

TABLE 36
Compositions for edaravone liquid formulations of example 64-65.

| | Example No. (#) - Quantity (mg/mL) | | | |
|---|---|---|---|---|
| Ingredient | #64 | | #65 | |
| Edaravone | 21 | mg | 21 | mg |
| Sodium metabisulfite | 1 | mg | 1 | mg |
| Citric acid monohydrate | 2.5 | mg | 2.5 | mg |
| Sodium hydroxide (1N) | q.s. to pH 4 | | q.s. to pH 5 | |
| Water (total) | 0.4 | mL | 0.4 | mL |

| | | |
|---|---|---|
| PEG 400 | q.s. to 1 mL | q.s. to 1 mL |
| Final pH (post 20x dilution) | 5.2 | 5.5 |

TABLE 37
Stability test results for example 64-65.

| Example | | | | % Maximum | % Total |
|---|---|---|---|---|---|
| No. | Time Point | Visual Description | % Purity | Single Impurity | Impurities |
| 64 | Initial | Clear, colorless solution | 99.9 | 0.05 | 0.1 |
| | 40° C./1 M | Clear, colorless solution | 99.6 | 0.15 | 0.4 |
| 65 | Initial | Clear, colorless solution | 99.9 | 0.05 | 0.1 |
| | 40° C./1 M | Clear, colorless solution | 99.5 | 0.26 | 0.5 |

Example 66 to 69 describes the composition of liquid formulation of edaravone, prepared using ingredients detailed in Table 38. Batch required quantity of PEG was added in required quantity of propylene glycol and mixed to dissolve or homogenized. Required quantity of edaravone was added in PG/PEG mixture under nitrogen overlay until it was dissolved and clear solution was obtained. Sodium metabisulfite was dissolved separately in partial quantity of nitrogen sparged water and added to edaravone solution. The volume of the batches was brought up to batch size using nitrogen sparged water and mixed to homogeneity. The solution was filled in to USP type I glass container and sealed with closure using nitrogen overlay. The stability test results for example 66-69 are provided in Table 41.

TABLE 38
Compositions for edaravone liquid formulations of example 66-69.

| | Example No. (#) - Quantity (mg/mL) | | | | | | | |
|---|---|---|---|---|---|---|---|---|
| Ingredient | #66 | | #67 | | #68 | | #69 | |
| Edaravone | 21 | mg | 21 | mg | 21 | mg | 21 | mg |
| Sodium metabisulfite | 1 | mg | 1 | mg | 1 | mg | 1 | mg |
| Polyethylene glycol 200 | 0.3 | mL | — | | — | | — | |
| Polyethylene glycol 300 | — | | 0.3 | mL | — | | — | |
| Polyethylene glycol 400 | — | | — | | 0.3 | mL | — | |
| Polyethylene glycol 4000 | — | | — | | — | | 0.3 | g |
| Propylene glycol | 0.5 | mL | 0.5 | mL | 0.5 | mL | 0.5 | mL |

| | | | | |
|---|---|---|---|---|
| Water | q.s. to 1 mL | q.s. to 1 mL | q.s. to 1 mL | q.s. to 1 mL |

Example 70 to 76 describe the composition of liquid formulation of edaravone, prepared using ingredients detailed in Table 39 and Table 40. In first step, citrate buffer was prepared by dissolving required quantity of citric acid, and (where applicable) sodium metabisulfite, and L-cysteine HCl in about 70% of the required quantity of nitrogen sparged water. pH was slowly adjusted to 4 using dilute sodium hydroxide. In second step, edaravone solution was prepared separately. Batch required quantity of PEG was added in required quantity of propylene glycol and mixed to dissolve or homogenize. Where applicable, sucralose, methylparaben, and propylparaben were added in PG/PEG mixture and mixed until dissolved. Required quantity of edaravone was added in PG/PEG mixture and mixed until dissolved under nitrogen overlay to obtain clear solution. Previously prepared citrate buffer was added in edaravone solution and mixed to homogeneity. The volume of the batches was brought up to batch size using nitrogen sparged water and mixed to homogeneity. The solution was filled in to USP type I glass container and sealed with closure using nitrogen overlay. The stability test results for example 70-76 are provided in Table 41.

TABLE 39
Compositions for edaravone liquid formulations of example 70-73.

| | Example No. (#) - Quantity (mg/mL) | | | | | | | |
|---|---|---|---|---|---|---|---|---|
| Ingredient | #70 | | #71 | | #72 | | #73 | |
| Edaravone | 21 | mg | 21 | mg | 21 | mg | 21 | mg |
| Citric acid monohydrate | — | | — | | 2.5 | mg | 2.5 | mg |
| Sodium hydroxide (1N) | — | | — | | q.s. to pH 4 | | q.s. to pH 4 | |
| L-cysteine HCl monohydrate | 0.1 | mg | 0.1 | mg | — | | — | |
| Sodium metabisulfite | 1 | mg | 1 | mg | 1 | mg | 1 | mg |
| Polyethylene glycol 400 | 0.3 | mL | — | | 0.3 | mL | — | |
| Polyethylene glycol 4000 | — | | 0.3 | g | — | | 0.3 | g |
| Propylene glycol | 0.5 | mL | 0.5 | mL | 0.5 | mL | 0.5 | mL |

| | | | | |
|---|---|---|---|---|
| Water | q.s. to 1 mL | q.s. to 1 mL | q.s. to 1 mL | q.s. to 1 mL |

TABLE 40
Compositions for edaravone liquid formulations of example 74-76.

| | Example No. (#) - Quantity (mg/mL) | | | | | |
|---|---|---|---|---|---|---|
| Ingredient | #74 | | #75 | | #76 | |
| Edaravone | 21 | mg | 21 | mg | 21 | mg |
| Citric acid monohydrate | 2.5 | mg | 2.5 | mg | 2.5 | mg |
| Sodium hydroxide (1N) | q.s. to pH 4 | | q.s. to pH 4 | | q.s. to pH 4 | |
| L-cysteine HCl monohydrate | 0.1 | mg | 0.1 | mg | — | |
| Sodium metabisulfite | 1 | mg | 1 | mg | 1 | mg |
| Sucralose | — | | — | | 5 | mg |
| Methylparaben | — | | — | | 2.5 | mg |
| Propylparaben | — | | — | | 0.25 | mg |
| Polyethylene glycol 400 | 0.3 | mL | — | | 0.3 | mL |
| Polyethylene glycol 4000 | — | | 0.3 | g | 0.25 | g |
| Propylene glycol | 0.5 | mL | 0.5 | mL | 0.5 | mL |

| | | | |
|---|---|---|---|
| Water | q.s. to 1 mL | q.s. to 1 mL | q.s. to 1 mL |

TABLE 41
Stability test results for example 66-76.

| Example | | | | % Maximum | % Total |
|---|---|---|---|---|---|
| No. | Time Point | Visual Description | % Purity | Single Impurity | Impurities |
| 66 | Initial | Clear, colorless solution | 99.7 | 0.09 | 0.3 |
| | 40° C./1 M | Clear, colorless solution | 98.6 | 0.27 | 1.4 |
| 67 | Initial | Clear, colorless solution | 99.6 | 0.11 | 0.4 |
| | 40° C./1 M | Clear, colorless solution | 98.5 | 0.21 | 1.5 |
| 68 | Initial | Clear, colorless solution | 99.9 | 0.05 | 0.1 |
| | 40° C./1 M | Clear, colorless solution | 99.7 | 0.11 | 0.3 |
| 69 | Initial | Clear, colorless solution | 99.9 | 0.04 | 0.1 |
| | 40° C./1 M | Clear, colorless solution | 99.3 | 0.44 | 0.7 |
| 70 | Initial | Clear, colorless solution | 99.9 | 0.05 | 0.1 |
| | 40° C./1 M | Clear, colorless solution | 99.7 | 0.07 | 0.3 |
| 71 | Initial | Clear, colorless solution | 99.9 | 0.04 | 0.1 |
| | 40° C./1 M | Clear, colorless solution | 99.3 | 0.48 | 0.7 |
| 72 | Initial | Clear, colorless solution | 99.9 | 0.04 | 0.1 |
| | 40° C./1 M | Clear, colorless solution | 99.7 | 0.09 | 0.1 |
| 73 | Initial | Clear, colorless solution | 99.9 | 0.04 | 0.1 |
| | 40° C./1 M | Clear, colorless solution | 99.2 | 0.49 | 0.8 |
| 74 | Initial | Clear, colorless solution | 99.9 | 0.04 | 0.1 |
| | 40° C./1 M | Clear, colorless solution | 99.7 | 0.07 | 0.3 |
| 75 | Initial | Clear, colorless solution | 99.9 | 0.04 | 0.1 |
| | 40° C./1 M | Clear, colorless solution | 99.3 | 0.50 | 0.7 |
| 76 | Initial | Clear, colorless solution | — | — | — |
| | 40° C./1 M | Clear, colorless solution | 99.3 | 0.47 | 0.7 |

### Prophetic Examples

Following are the additional examples which were not prepared by the inventor but could be prepared based on the present disclosure.

Example 77 to 80 describe liquid formulation of edaravone that can be prepared using compositions provided in Table 42. In first step, citrate buffer is prepared by dissolving required quantity of citric acid, and (where applicable) sodium metabisulfite, and L-cysteine HCl in about 700% of the required quantity of nitrogen sparged water. Then pH is slowly adjusted to 4 using dilute sodium hydroxide. In second step, edaravone solution is prepared separately. Batch required quantity of PEG is added in required quantity of propylene glycol and mixed to dissolve or homogenize. Where applicable, sucralose, peppermint flavor are added in PG/PEG mixture and mixed until dissolved. Required quantity of edaravone is added in PG/PEG mixture and mixed until dissolved under nitrogen overlay to obtain clear solution. Citrate buffer is then added in edaravone solution and mixed to homogeneity. The volume of the batches is brought up to batch size using nitrogen sparged water and mixed to homogeneity. The solution is filled in to appropriate container and sealed with closure using nitrogen overlay.

TABLE 42
Compositions for edaravone liquid formulations of example 77-80.

| | Example No. (#) - Quantity (mg/mL) | | | | | | | |
|---|---|---|---|---|---|---|---|---|
| Ingredient | #77 | | #78 | | #79 | | #80 | |
| Edaravone | 21 | mg | 30 | mg | 40 | mg | 40 | mg |
| Citric acid monohydrate | 2.5 | mg | 2.5 | mg | 2.5 | mg | 2.5 | mg |
| Sodium hydroxide | q.s. to pH 4 | | q.s. to pH 4 | | q.s. to pH 4 | | q.s. to pH 4 | |
| L-cysteine HCl monohydrate | 0.1 | mg | 0.1 | mg | — | | 0.5 | mg |
| Sodium metabisulfite | 1 | mg | 2 | mg | 2 | mg | 2 | mg |
| Sucralose | 25 | mg | 25 | mg | 25 | mg | 25 | mg |
| Peppermint flavor | 1 | mg | 5 | mg | 5 | mg | 5 | mg |
| Polyethylene glycol 400 | 0.3 | mL | 0.3 | mL | 0.5 | mL | 0.5 | mL |
| Propylene glycol | 0.5 | mL | 0.5 | mL | 0.3 | mL | 0.3 | mL |

| | | | | |
|---|---|---|---|---|
| Water | q.s. to 1 mL | q.s. to 1 mL | q.s. to 1 mL | q.s. to 1 mL |

1. A stable liquid pharmaceutical formulation comprising:
(a) edaravone at a concentration from about 10 mg/mL to about 50 mg/mL;
(b) one or more pharmaceutically acceptable excipients; and
(c) a pharmaceutically acceptable vehicle comprising:
(i) propylene glycol, polyethylene glycol, glycerin or combination thereof, and
(ii) less than about 10% w/v of ethanol;
wherein the formulation is a single-phase solution essentially free from visible particles; and
wherein upon storage for at least three months at room temperature, the edaravone in the formulation has a purity of at least about 95%.

2. The formulation according to claim 1, wherein the pharmaceutically acceptable vehicle comprises propylene glycol, polyethylene glycol, glycerin or a combination thereof at a concentration from about 40% w/v to about 90% w/v.

3. The formulation according to claim 1, wherein the edaravone concentration is about 21 mg/mL.

4. The formulation according to claim 1, wherein the excipient is selected from the group comprising of vehicles, preservatives, sweeteners, flavors, pH adjusting agents, buffering agents, viscosity adjusting agents, antioxidants, tonicity adjusting agents, chelating agents, permeability enhancing agents, mucoadhesive agents, stabilizers, complexing agents, crystallization inhibitors, suspending agents, surfactants, anti-foaming agents, dispersing agents, wetting agents, emulsifying agents, and combinations thereof.

5. The formulation according to claim 1, wherein upon storage for at least six months at room temperature, the edaravone in the formulation has a purity of at least about 98%.

6. The formulation according to claim 1, wherein the formulation is suitable for administration selected from oral, sublingual, buccal, injectable, or intranasal routes.

7. The formulation according to claim 1, wherein molecular weight of polyethylene glycol is from about 200 to about 4000.

8. The formulation according to claim 1, wherein the formulation comprises water at concentration from about 1% to about 50%.

9. The formulation according to claim 1, wherein the formulation has a pH from about 3 to about 6.

10. The formulation according to claim 1, comprising at least one antioxidant selected from a pharmaceutically acceptable bisulfite or metabisulfite salt (including sodium bisulfite, potassium bisulfite, ammonium bisulfite, sodium metabisulfite, or potassium metabisulfite).

11. The formulation according to claim 1, comprising at least one stabilizer selected from L-cysteine or a pharmaceutically acceptable salt or hydrate thereof.

12. The formulation according to claim 1, wherein the formulation is filled in a container with an overlay of an inert gas selected from nitrogen or argon.

13. The formulation according to claim 1, formulated as an oral liquid comprising: edaravone at about 1.5% w/v to about 2.5% w/v; propylene glycol, polyethylene glycol, glycerin or combination thereof at concentration of about 40% w/v to about 99% w/v; at least one antioxidant at concentration from about 0.001% w/v to about 0.5% w/v; and water.

14. The formulation according to claim 1, wherein the formulation is filled into a multi-dose or unit-dose glass or polymeric container having a fill volume of about 1 mL to about 500 mL.

15. The formulation according to claim 14, wherein the container is provided with a flow-restrictor and dosing syringe.

16. A method for treating a condition selected from the group consisting of amyotrophic lateral sclerosis (ALS), acute ischemic stroke (AIS), Alzheimer's disease (AD), autism spectrum disorder (ASD), acute mountain sickness (AMS), hearing loss/vestibular disorders, contrast-induced acute kidney injury (CI-AKI), Parkinson's disease (PD), and traumatic brain injury (TBI), the method comprising administering a therapeutically effective amount of the formulation according to claim 1 to a patient in need thereof.

17. A method according to claim 16, wherein the condition is amyotrophic lateral sclerosis (ALS).

18. The method according to claim 16, wherein the formulation is administered orally in a volume of about 1 mL to about 20 mL to deliver about 5 mg to about 200 mg of edaravone per dose.

19. A method of preparing the liquid pharmaceutical formulation according to claim 1, the method comprising the steps of: (i) solubilizing one or more optional pharmaceutically acceptable excipients in a vehicle; (ii) solubilizing edaravone; (iii) optionally filtering the formulated liquid; (iv) filling the filtered liquid into a primary packaging container; and (v) sealing the container with an inert gas overlay.

20. A stable oral liquid pharmaceutical formulation comprising: (a) edaravone at a concentration of about 15 mg/mL to about 25 mg/mL; (b) a vehicle comprising propylene glycol, polyethylene glycol, or a mixture thereof, present at a concentration of at least about 50% w/v; (c) one or more stabilizers selected from the group consisting of L-cysteine, pharmaceutically acceptable salt of bisulfite, or combinations thereof; and (d) water; wherein the formulation provides a unit dose of about 105 mg of edaravone in a total volume of about 5 mL.

21. The formulation according to claim 20, wherein the vehicle comprises propylene glycol or polyethylene glycol; and wherein the formulation exhibits less than about 1.0% total impurities after storage at 121° C. for 20 minutes.
